(19) United States
(12) Patent Application Publication
(10) Pub. No.: US 2018/0215777 A1
(43) Pub. Date: Aug. 2, 2018

(54) COMPLEXES FOR INTRACELLULAR IMAGING

(71) Applicants: UNIVERSITY OF SOUTH AUSTRALIA, Adelaide, South Australia (AU); CURTIN UNIVERSITY, Perth, Western Australia (AU)

(72) Inventors: Doug Brooks, Cheltenham (AU); Sally Plush, Mile End (AU); Massimiliano Massi, Wilson (AU); Stefano Stagni, Bologna (IT)

(21) Appl. No.: 15/748,336

(22) PCT Filed: Jul. 29, 2016
(86) PCT No.: PCT/AU2016/050685

§ 371 Date: Jan. 29, 2018

(30) Foreign Application Priority Data
Jul. 30, 2015 (AU) 2015903041

(51) Int. Cl. C07H 23/00; G01N 33/58; A61K 49/00; G01N 33/574

(52) U.S. Cl. CPC C07H 23/00; G01N 33/57434; A61K 49/0019; G01N 33/582

(57) ABSTRACT

The present disclosure relates to complexes for intracellular imaging and also to methods and kits for intracellular imaging or cell labelling. Certain embodiments of the present disclosure provide a complex comprising a transition metal carbonyl compound, a conjugated bidentate ligand and a tetrazolato compound comprising a saccharide group.

### PRIORITY CLAIM

This application claims priority to Australian provisional patent application number 2015903041 filed on 30 Jul. 2015, the content of which is hereby incorporated by reference.

### FIELD

The present disclosure relates to complexes for intracellular imaging and also to methods and kits for intracellular imaging or cell labelling.

### BACKGROUND

Molecular imaging agents are important tools in both research and medical diagnosis. A variety of different types of imaging agents are available. Some of these agents target specific types of biological species, such as DNA, RNA or proteins, while other imaging agents can be used to target specific cells or target specific cell structures, such as the use of antibodies that bind to cells, or antibodies or small molecules specific to proteins associated with certain cell structures.

Some imaging agents are only suitable for cells that have been fixed or are no longer viable, while other imaging agents may be used on live or dead cells.

A wide variety of imaging agents are available that can be used to target live cells by binding to cell surface markers. However, the suite of agents that can be used for intracellular imaging of cells is more limited. Agents that have the ability to be used on live cells and which also label or detect intracellular components provide a number of advantages.

Indeed, the identification of agents that are suitable for live cell imaging has become an important area for development. The ability to intracellular image cells not only has important implications for visualizing normal cell function, but also has direct significance for the investigation of many diseases. Such agents also have the potential to provide diagnostic or prognostic tools that can be applied to discern specific patient pathologies.

As such, there is a need for the identification of new agents that have the ability to intracellularly image cells. The limited range of intracellular imaging agents available continues to be a hindrance to progress in this area.

Accordingly, for a variety of reasons there is a need for new agents that can be used for the intracellular imaging of cells.

### SUMMARY

The present disclosure relates to complexes for intracellular imaging and also to methods and kits for intracellular imaging.

Certain embodiments of the present disclosure provide a complex comprising a transition metal carbonyl compound, a conjugated bidentate ligand and a tetrazolato compound comprising a saccharide group.

Certain embodiments of the present disclosure provide a complex with one of the following chemical structures:

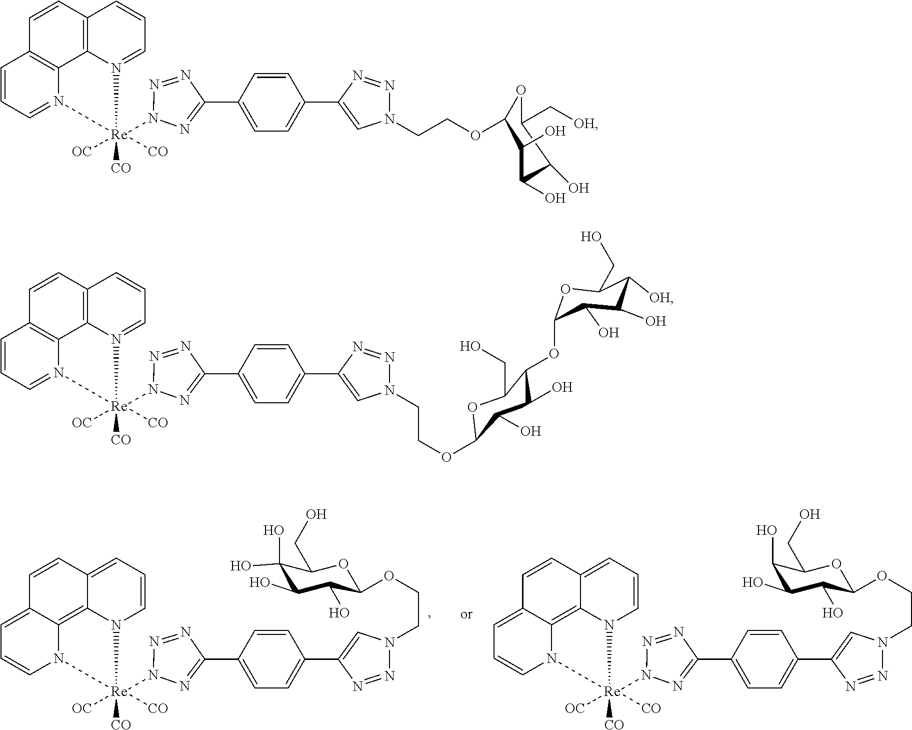

and/or a salt, a solvate, a tautomer, a stereoisomer, a substituted derivative or an open chain form of the saccharide of any of the chemical structures.

Certain embodiments of the present disclosure provide an intracellular imaging agent, the agent comprising a complex comprising a transition metal carbonyl compound, a conjugated bidentate ligand and a tetrazolato compound comprising a saccharide group.

Certain embodiments of the present disclosure provide a method of intracellular imaging of a cell, the method comprising exposing a cell to a complex comprising a transition metal carbonyl compound, a conjugated bidentate ligand and a tetrazolato compound comprising a saccharide group, and imaging the complex in the cell.

Certain embodiments of the present disclosure provide a method of labelling or detecting a vesicular structure in a cell, the method comprising exposing the cell to a complex with the following chemical structure:

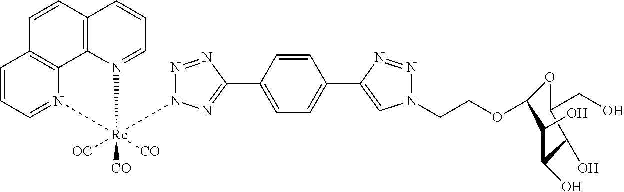

and/or a salt, solvate, tautomer, a stereoisomer, a substituted derivative, or an open chain form of the saccharide thereof, and thereby labelling or detecting a vesicular structure in the cell.

Certain embodiments of the present disclosure provide a method of labelling or detecting endoplasmic reticulum and/a lysosome in a cell, the method comprising exposing the cell to a complex with the following chemical structure:

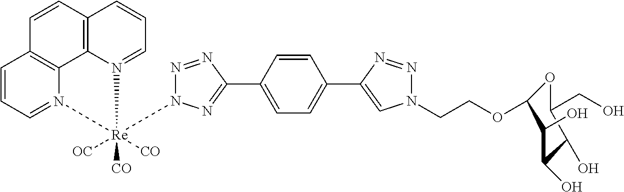

and/or a salt, a solvate, a tautomer, a stereoisomer, a substituted derivative, or an open chain form of the saccharide, and detecting or labelling the endoplasmic reticulum and/or the lysosome in the cell.

Certain embodiments of the present disclosure provide a method of labelling or detecting cytosol and/or a cytosolic structure in a cell, the method comprising exposing a cell to a complex with the following chemical structure:

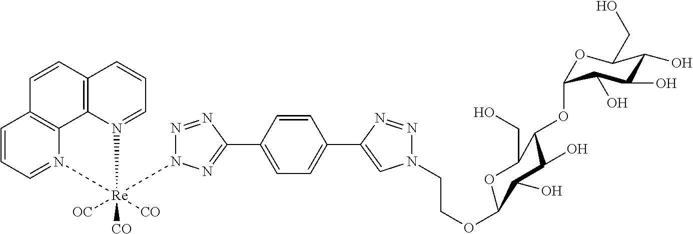

and/or a salt, a solvate, a tautomer, a stereoisomer, a substituted derivative, or an open chain form of the saccharide, and thereby labelling or detecting cytosol or a cytosolic structure in the cell.

Certain embodiments of the present disclosure provide a method of identifying a cancerous prostate cell, the method comprising exposing the cell to a complex with the following chemical structure:

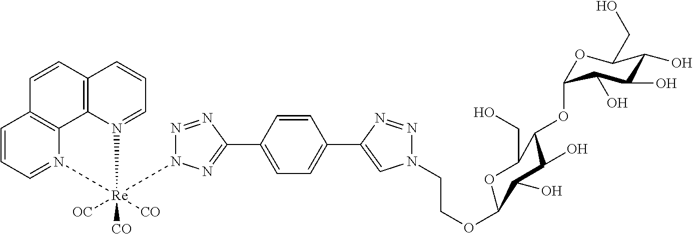

and/or a salt, a solvate, a tautomer, a stereoisomer, a substituted derivative, or an open chain form of the saccharide thereof, and identifying the cell as a cancerous prostate cell on the basis of altered labelling of the cell with the complex and/or altered localisation of the complex in the cell.

Certain embodiments of the present disclosure provide a method of screening for a cancerous prostate cell, the method comprising exposing the cell to a complex with the following chemical structure:

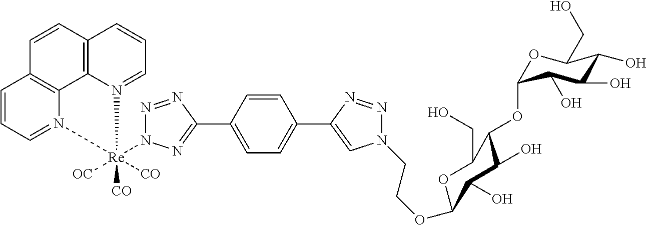

and/or a salt, a solvate, a tautomer, a stereoisomer, a substituted derivative, or an open chain form of the saccharide, and identifying the cell as a cancerous prostate cell on the basis of one or more of increased labelling of the cell with the complex, cytoplasmic labelling of the cell with the complex, localisation of the complex in punctate structures in the cytosol of the cell and increased perinuclear localisation of the complex in the cell.

Certain embodiments of the present disclosure provide a method of identifying a prostate cancer in a subject, the method comprising exposing a cell from the subject to a complex with the following chemical structure:

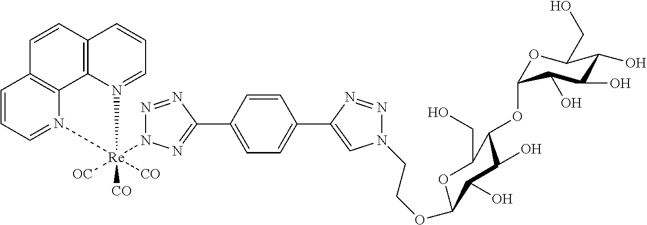

and/or a salt, a solvate, a tautomer, a stereoisomer, a substituted derivative, or an open chain form of the saccharide, and identifying prostate cancer in the subject on the basis of altered labelling of the cell with the complex and/or altered localisation of the complex in the cell.

Certain embodiments of the present disclosure provide a method of identifying a prostate cancer in a subject, the method comprising exposing the subject to a complex with the following chemical structure:

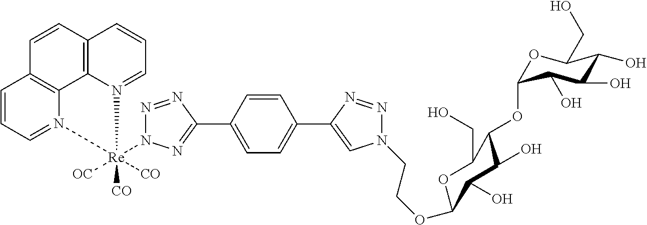

and/or a salt, a solvate, a tautomer, a stereoisomer, a substituted derivative, or an open chain form of the saccharide thereof, and identifying prostate cancer in the subject on the basis of altered labelling of prostate cells with the complex and/or altered localisation of the complex in prostate cells in the subject.

Certain embodiments of the present disclosure provide a method for labelling or detecting endoplasmic reticulum and/or other cellular structures in a cell, the method comprising exposing a cell to a complex with the following chemical structure:

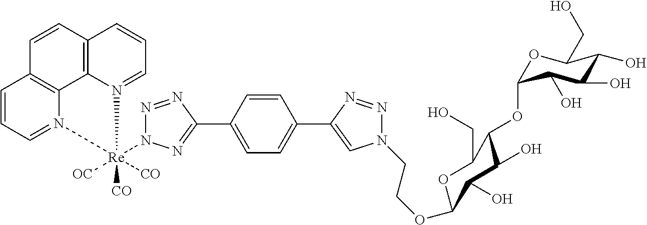

and/or a salt, a solvate, a tautomer, a stereoisomer, a substituted derivative, or an open chain form of the saccharide, and thereby labelling or detecting endoplasmic reticulum and other cellular structures in the cell.

Certain embodiments of the present disclosure provide a kit for intracellular imaging of cells, the kit comprising a complex comprising a transition metal carbonyl compound, a conjugated bidentate ligand and a tetrazolato compound comprising a saccharide group.

Certain embodiments of the present disclosure provide a method of identifying a complex for intracellular imaging of a cell, the method comprising:
providing a candidate complex comprising a transition metal carbonyl compound, a conjugated bidentate ligand and a tetrazolato compound comprising a saccharide group;
determining the ability of the candidate complex to intracellularly label a cell; and
identifying the candidate complex as a complex for intracellular imaging of a cell.

Certain embodiments of the present disclosure provide a method of producing a complex comprising a transition metal carbonyl compound, a conjugated bidentate ligand and a tetrazolato compound comprising a saccharide group, the method comprising:
providing a complex comprising a transition metal carbonyl compound, a conjugated bidentate ligand and a tetrazolato compound and reacting the tetrazolato compound with a saccharide and/or a derivative thereof; or
providing a transition metal carbonyl compound, a conjugated bidentate ligand and a tetrazolato compound comprising a saccharide group and forming a complex from the aforementioned components; and
thereby producing a complex comprising a transition metal carbonyl compound, a conjugated bidentate ligand and a tetrazolato compound comprising a saccharide group.

Certain embodiments of the present disclosure provide a complex with the following formula:

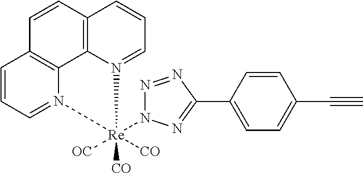

or salt thereof, a solvate thereof, a stereoisomer thereof, or a substituted derivative thereof.

Other embodiments are disclosed herein.

### BRIEF DESCRIPTION OF THE FIGURES

Certain embodiments are illustrated by the following figures. It is to be understood that the following description is for the purpose of describing particular embodiments only and is not intended to be limiting with respect to the description.

FIG. 1 shows isomers of a neutral Rhenium(I) complex.

FIG. 2 shows a neutral Rhenium(I) tetrazolato complex.

FIG. 3 shows the reaction of a 1,3-dipolar cycloaddition forming a 1,4- or 1,5-triazole linker

FIG. 4 shows Scheme 1 for the synthesis of azido mannose and azido maltose residues.

FIG. 5 shows Scheme 2 for the synthesis of targeted Rhenium tetrazolato complexes.

FIG. 6 shows UV-Visible absorption spectra of Re2 in 100% H₂O with varying pH.

FIG. 7 shows a change in molar absorbance at 405 nm for Re2 in 100% H₂O over the pH range of 3-8.

FIG. 8 shows fluorescence spectra of Re2 in 100% H₂O with varying pH.

FIG. 9 shows the change in Re2 fluorescence at 552 nm over the pH range of 3-8.

FIG. 10 shows the UV-Visible absorption spectra of Re2 in 50:50 MeOH/H₂O with varying pH.

FIG. 11 shows the change in molar absorbance for Re2 in 50:50 MeOH/H₂O over the pH range of 2-10.

FIG. 12 shows fluorescence spectra of Re2 (256 nm excitation) in 50:50 MeOH/H₂O over the pH range of 2-10.

FIG. 13 shows the change in Re2 fluorescence (256 nm excitation) at 310, 398, 418, 440 and 560 nm in 50:50 MeOH/H₂O over the pH range of 2-10.

FIG. 14 shows fluorescence spectra of Re2 (275 nm excitation) in 50:50 MeOH/H₂O over the pH range of 2-10.

FIG. 15 shows the change in Re2 fluorescence (275 nm excitation) at 310, 345 and 560 nm in 50:50 MeOH/H₂O over the pH range of 2-10.

FIG. 16 shows fluorescence spectra of Re2 (366 nm excitation) in 50:50 MeOH/H₂O over the pH range of 2-10.

FIG. 17 shows changes in Re2 fluorescence (366 nm excitation) at 418, 440 and 560 nm in 50:50 MeOH/H₂O over the pH range of 2-10.

FIG. 18 shows fluorescence spectra of Re2 (405 nm excitation) in 50:50 MeOH/H₂O over the pH range of 2-10.

FIG. 19 shows change in Re2 fluorescence (405 nm excitation) at 555 nm in 50:50 MeOH/H₂O over the pH range of 2-10.

FIG. 20 shows UV-Visible absorption spectra of Re3 in 50:50 MeOH/H2O with varying pH.

FIG. 21 shows change in molar absorbance for Re3 in 50:50 MeOH/H2O over the pH range of 2-10.

FIG. 22 shows fluorescence spectra of Re3 (256 nm excitation) in 50:50 MeOH/H₂O over the pH range of 2-10.

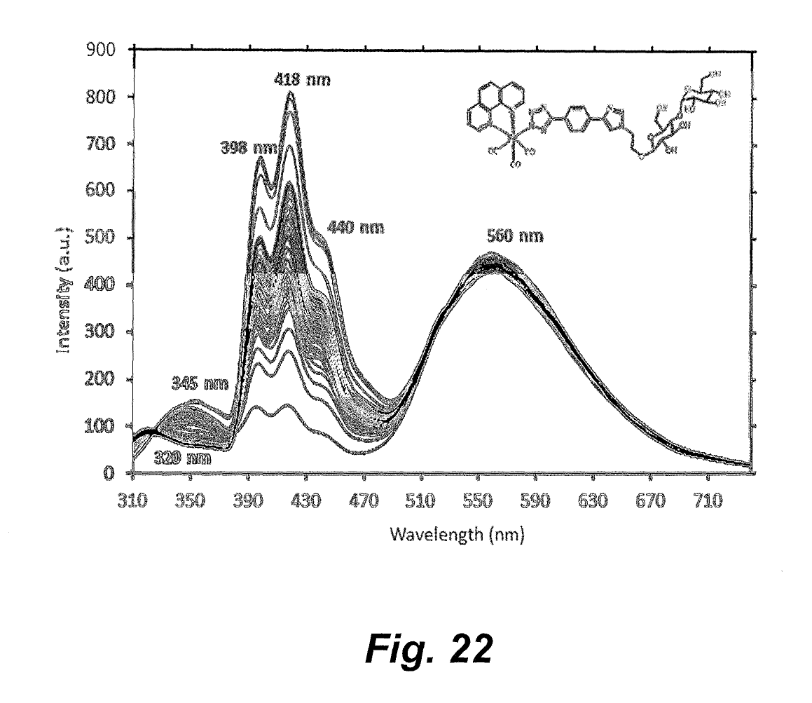

FIG. 23 shows change in Re3 fluorescence (256 nm excitation) at 320, 398, 418, 440 and 560 nm in 50:50 MeOH/H₂O over the pH range of 2-10.

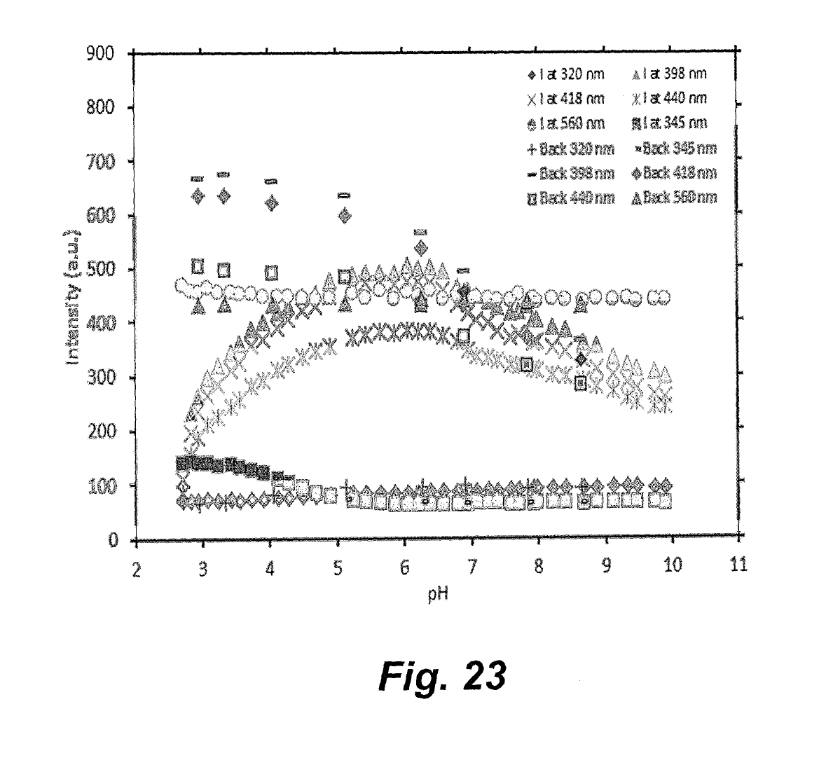

FIG. 24 shows fluorescence spectra of Re3 (275 nm excitation) in 50:50 MeOH/H₂O over the pH range of 2-10.

FIG. 25 shows change in Re3 fluorescence (275 nm excitation) at 320, 345 and 560 nm in 50:50 MeOH/H₂O over the pH range of 2-10.

FIG. 26 shows fluorescence spectra of Re3 (366 nm excitation) in 50:50 MeOH/H₂O over the pH range of 2-10.

FIG. 27 shows change in Re3 fluorescence (366 nm excitation) at 418, 440 and 560 nm in 50:50 MeOH/H₂O over the pH range of 2-10.

FIG. 28 shows fluorescence spectra of Re3 (405 nm excitation) in 50:50 MeOH/H₂O over the pH range of 2-10.

FIG. 29 shows change in Re3 fluorescence (405 nm excitation) at 555 nm in 50:50 MeOH/H₂O over the pH range of 2-10.

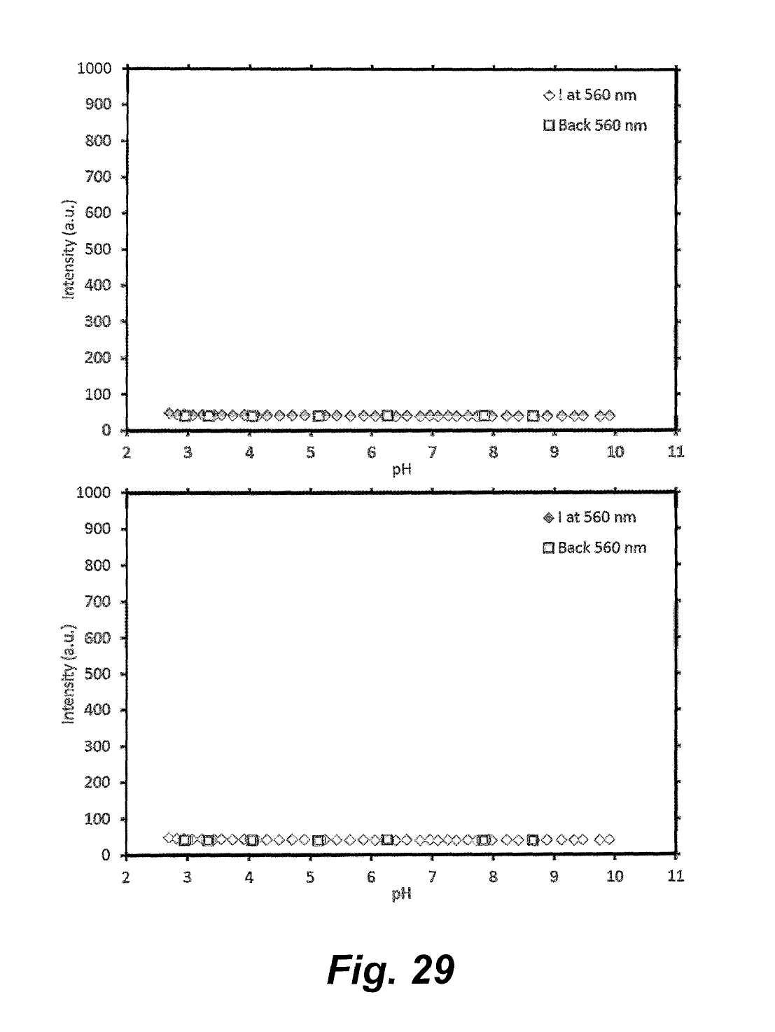

FIG. 30 shows cellular distribution of Re2 (Rhenium mannose) (100 μm) in live Drosophila fat body cells.

FIG. 31 shows Re2 (Rhenium mannose) (100 μM) colocalised with LAMP-GFP in Drosophila fat body cells.

FIG. 32 shows cellular distribution of Re3 (Rhenium maltose) (100 μm) in live Drosophila fat body cells.

FIG. 33 shows cellular distribution of Re3 (Rhenium maltose) (100 μm) in THP-1 macrophages.

FIG. 34 shows cellular distribution of Re3 (Rhenium maltose) (100 μm) in control prostate cells (PNT1a, PNT2) and prostate cancer cells (Du145, 22RV1, LNCaP).

FIG. 35 shows distribution of the probe Re2 in live prostate cancer cells. (A-D) Micrographs of cross-section through the prostate cells showing intracellular localisation of Re2 and in secretory compartments. Representative images were from live (A) non-malignant control PNT1a and prostate cancer (B) 22RV1, (C) LNCaP and (D) DU145 cell lines. Arrows depict cell surface associated Re2-positive compartments. Scale bar, 20 μm.

FIG. 36 shows Re2 localises to the endoplasmic reticulum in prostate cells. (A-D) Confocal micrographs showing co-localisation of the probe Re2 (green in A-D, greyscale in A/-D/) and ER-Tracker™ (red in A-D, greyscale in A//-D//) in prostate cell lines. Representative images were from live (A) non-malignant control PNT1a and prostate cancer (B) 22RV1, (C) LNCaP and (D) DU145 cell lines. Scale bar, 20 μm.

FIG. 37 shows Re2 accumulates in lysosomes in prostate cancer 22RV1 cells. Micrograph of the cross-section through the 22RV1 cells showing co-localisation of Re2 and LysoTracker® Red DND-99. Arrows depict lysosomes containing Re2. Scale bar, 20 μm.

FIG. 38 shows that Re2 accumulates in cell surface associated compartments of prostate cells. (A-D) Micrographs of cross-section through prostate non-malignant control PNT1a (A, A/) and cancer (B, B/—22RV1; C, C/—LNCaP; and D, D/—DU145) cells showing Re2 (green in A-D and greyscale in A/-D/) in the extracellular compartments. The plasma membrane was outlined by CellMask deep Red (red in A-D). Arrows depict cell surface associated compartments. Scale bar, 20 μm.

FIG. 39 shows Re2 accumulates in cell surface associated vesicles and lysosomes in live THP-1 macrophages. (A-A^{//}). Micrographs of cross-section through macrophages showing accumulation of Re2 (green in A, greyscale in A^{/}) in cell surface associated vesicles, where cell membrane was outlined by CellMask™ Deep Red (red in A, greyscale in A^{//}). Arrow in A-A^{///} depicts these Re2-positive cell surface associated vesicles. (B-B^{//}). Micrographs of cross-section through THP-1 macrophages showing co-localisation of Re2 (green in A, greyscale in A^{/}) and LysoTracker® Red DND-99 (red in B, greyscale in B^{//}red). Arrow in B-B^{///} shows lysosomes in which Re2 has been observed. Brightfield images of THP-1 macrophages are in A^{///} and B^{///}. Scale bar, 20 μm.

FIG. 40 shows the subcellular localisation of Re3. (A) Confocal micrograph showing unstained H9c2 cells. (B-E) Micrographs of cross-section through H9c2 rat cardiomyoblasts showing intracellular distribution of Re3 in. (B, C) The cells were stained with Re3 in D-glucose-free and FCS-free media for 30 minutes. (D, E) The cells were stained with the probe in media containing D-glucose. The H9c2 cells were imaged after they were washed from the probe (C, E) and prior to it (B, D). Scale bar, 20 μm.

FIG. 41 shows Re3 localises to the endoplasmic reticulum in H9c2 cells. Confocal micrographs showing co-localisation of the probe Re3 and ER-Tracker™ in the H9C2 rat cardiomyoblasts. Scale bar, 20 μm.

FIG. 42 shows Re3 imaging of infarction in lamb. Micrographs of cross-section through the myocardium showing distribution of Re3. Representative images were from healthy (A) and infarcted myocardium (B) excised from lambs. Scale bar, 50 μm.

FIG. 43 shows confocal micrographs for intracellular localisation of Re2 in lamb quadriceps. Representative images were from healthy lambs. Scale bar, 20 μm.

FIG. 44 shows the details of reaction Scheme 3.

FIG. 45 shows the details of reaction Scheme 4.

### DETAILED DESCRIPTION

The present disclosure relates to complexes for intracellular imaging and cell labelling, and also to methods and kits for intracellular imaging and/or cell labelling.

Certain embodiments of the present disclosure are directed to products, methods and kits that have one or more combinations of advantages. For example, some of the advantages of the embodiments disclosed herein include one or more of the following: new agents for labelling or imaging cells; new agents for intracellular imaging of cells; new agents for intracellular imaging of live cells; new agents that are localised or distributed in specific cell structures or compartments; new agents that are localised in lysosomes; new agents that are localised or distributed through the endoplasmic reticulum; new agents that are localised or distributed through the cytoplasm; agents that show altered cellular distribution in prostate cancer cells; reagents for investigating cell biology or cell function; agents that resist photobleaching; to address one or more problems and/or to provide one or more advantages, or to provide a commercial alternative. Other advantages of certain embodiments of the present disclosure are also disclosed herein.

The present disclosure is based on the recognition that Re(I) complexes of a transition metal carbonyl compound, a bidentate ligand and a tetrazolato compound functionalised with a saccharide group are suitable for cellular imaging, including imaging of a live cells. In addition, it has been determined that some of these agents have specificity for some intracellular structures or compartments, demonstrating their utility as probes for labelling or imaging of cells. Further, some of these agents show altered cellular distribution in prostate cancer cells.

Certain embodiments of the present disclosure provide a complex comprising a transition metal carbonyl compound, a conjugated bidentate ligand and a tetrazolato compound comprising a saccharide group.

Complexes (sometimes referred herein to as “probes”) comprising a transition metal carbonyl compound, a conjugated bidentate ligand and a tetrazolato compound comprising a saccharide group, may be produced as described herein.

Precursor to the complexes, for example those comprising a transition metal carbonyl compound, a conjugated bidentate ligand and a tetrazolato compound may be synthesized by a method known in the art, for example as described in Wright P. J. et al (2013) “Ligand-Induced Structural, Photophysical, and Electrochemical Variations in Tricarbonyl Rhenium(I) Tetrazolato Complexes” Organometallics 32: 3728-3737 and Wright P. J. et al (2013) “Synthesis, Photophysical and Electrochemical Investigation of Dinuclear Tetrazolato-Bridged Rhenium Complexes” Organometallics 31: 7566-7578. Methods of synthesis of transition metal carbonyl compounds and bidentate ligands are known in the art. Precursor compounds comprising a saccharide group may be synthesized as described herein, and coupled to a tetrazolato compound as described herein

It will be appreciated that the complexes as described herein include the complexes themselves, and/or a substituted form or a derivative thereof, an acceptable salt thereof, a solvate thereof, a hydrate thereof, a stereoisomer thereof, a tautomer thereof or a metabolite thereof. The term “substituted” refers to the substitution of one atom or functional group with another acceptable atom or functional group, such as the substitution of a hydrogen atom for another functional group such as a halogen atom, a hydroxyl group, a cyano group, an amine group, an ether group, an ester group, an amide group, a sulfonate group, a phosphate group, or alkyl group (eg a methyl group, an ethyl group, a hexyl group, a dodecyl group). Other types of substitutions are contemplated.

In certain embodiments, the transition metal carbonyl compound comprises a transition metal ion comprising Re(I). Other transition metals are contemplated. Examples of other transition metal ions include Iridium and Ruthenium (Ir(III), and Ru(II)). In certain embodiments, the transitional metal carbonyl compound is a Re(I) carbonyl compound.

In certain embodiments, the transition metal carbonyl compound comprises a transition metal tricarbonyl compound. In certain embodiments, the transition metal carbonyl compound comprises a transition metal dicarbonyl compound. In certain embodiments, the transition metal carbonyl compound comprises a transition metal monocarbonyl compound.

In certain embodiments, the transition metal carbonyl compound comprises a Re(I) tricarbonyl compound.

In certain embodiments, the conjugated bidentate ligand binding to the transitional metal ion comprises a ligand that binds to the metal centre via at least one nitrogen atom. In certain embodiments, the conjugated bidentate ligand comprises a ligand that binds to the metal centre via two nitrogen atoms. In certain embodiments, the conjugated bidentate ligand comprises a ligand that binds to the metal centre via one nitrogen atom and a second atom, the second atom being selected from oxygen, sulphur or phosphorous.

In certain embodiments, the conjugated bidentate ligand comprises an aromatic bidentate ligand. In certain embodiments, the conjugated bidentate ligand comprises a bidentate diimine ligand. In certain embodiments, the conjugated bidentate ligand comprises an aromatic diimine ligand.

In certain embodiments, the conjugated bidentate diimine ligand comprises a phenanthroline compound. In certain embodiments, the conjugated bidentate diimine ligand comprises a 1,10-phenanthroline and/or a substituted derivative thereof.

In certain embodiments, the conjugated bidentate diimine ligand comprises a bipyridine compound, such as a 2,2′-bipyridine and/or a substituted derivative thereof.

Methods for producing a tetrazolato compound comprising a saccharide group are as described herein. In certain embodiments, the compound is produced using a reaction of an azide group with an alkyne group, to attach (directly or indirectly) a saccharide group to the tetrazolato compound. In certain embodiments, the compound is produced using a reaction of an alkyne group on the tetrazolato compound with azide group on the saccharide to attach or couple (directly or indirectly) a saccharide group to the tetrazolato compound. In certain embodiments, the compound is produced using a reaction of an azide group on the tetrazolato compound with an alkyne group on the saccharide to attach or couple (directly or indirectly) a saccharide group to the tetrazolato compound. In certain embodiments, amide coupling chemistry may be used.

In certain embodiments, the tetrazolato compound comprises an aryltetrazolate and/or a substituted derivative thereof. In certain embodiments, the tetrazolato compound comprises a phenyltetrazolate and/or a substituted derivative thereof.

In certain embodiments, the tetrazolato compound comprises a heteroaryltetrazolate and/or a substituted derivative thereof. In certain embodiments, the tetrazolato compound comprises an alkyltetrazolate and/or a substituted derivative thereof. In certain embodiments, the tetrazolato compound comprises a phenyltetrazolate and/or a substituted derivative thereof.

In certain embodiments, the tetrazolato compound comprises a triazole and/or a substituted derivative thereof. In certain embodiments, the triazole is indirectly attached to the tetrazolato compound. In certain embodiments, the tetrazolato compound comprises a 1,2,3 triazole and/or a substituted derivative thereof. In certain embodiments, the tetrazolato compound comprises an alkyl triazole and/or a substituted derivative thereof. In certain embodiments, the tetrazolato compound comprises an ethyl triazole and/or a substituted derivative thereof.

In certain embodiments, the tetrazolato compound comprises a triazole phenyltetrazolate and/or a substituted derivative thereof. In certain embodiments, the tetrazolato compound comprises a 1,2,3 triazole phenyltetrazolate and/or a substituted derivative thereof.

In certain embodiments, the tetrazolato compound comprises an alkyl triazole phenyltetrazolate and/or a substituted derivative thereof. In certain embodiments, the tetrazolato compound comprises a 1-alkyl, 1,2,3 triazole phenyltetrazolate and/or a substituted derivative thereof. In certain embodiments, the tetrazolato compound comprises a 1-ethyl, 1,2,3 triazole phenyltetrazolate and/or a substituted derivative thereof.

In certain embodiments, the saccharide group is directly or indirectly attached to the tetrazolato compound via a glycosidic bond. In certain embodiments, the glycosidic bond comprises a bond with a hydroxyl group on the parent saccharide. In certain embodiments, the saccharide group is attached, directly or indirectly, to the tetrazolato compound using a reaction of an azide group with an alkyne group. In certain embodiments, the saccharide group is attached, directly or indirectly, to the tetrazolato compound using a reaction of an azide group on the saccharide with an alkyne group on the tetrazolato compound.

The term “saccharide” group refers to a group derived from a saccharide or a sugar, and which is indirectly or directly attached to a tetrazolato compound, and includes the saccharide alone or a derivative of a saccharide group with one or more additional modifications to the saccharide. For example, the term “saccharide” refers to a saccharide group (cyclic or linear), an open chain form of a saccharide, or a derivative of a saccharide, such as a deoxy sugar (eg a deoxy ribose, a deoxy galactose), an amino sugar (eg a glucosamine), an alkyl ether variant (eg a methyl glucopyranoside an N-glycoside (eg a nucleoside), or a substituted derivative thereof. Other types of modifications are contemplated.

In certain embodiments, the saccharide group is directly or indirectly attached to the tetrazolato compound via an alkyl triazole. In certain embodiments, the saccharide group is directly or indirectly attached to the tetrazolato compound via an alkyl 1,2,3 triazole. In certain embodiments, the saccharide group is directly or indirectly attached to the tetrazolato compound via a 1-alkyl, 1,2,3 triazole. In certain embodiments, the saccharide group is directly or indirectly attached to the tetrazolato compound via a 1-ethyl, 1,2,3 triazole. In certain embodiments, the saccharide group is attached to the tetrazolato compound via a 1-alkyl, 4-phenyl, 1,2,3 triazole and/or a substituted derivative thereof. In certain embodiments, the saccharide group is attached to the tetrazolato compound via an 1-ethyl, 4-phenyl, 1,2,3 triazole and/or a substituted derivative thereof.

In certain embodiments, the saccharide group is directly or indirectly attached to the tetrazolato compound via a C1 attachment. In certain embodiments, the saccharide group is directly or indirectly attached to the tetrazolato compound via a C2 attachment. In certain embodiments, the saccharide group is directly or indirectly attached to the tetrazolato compound using a N-glycosamine linkage.

In certain embodiments, the saccharide group comprises a monosaccharide group, a disaccharide group, an oligosaccharide group and/or a polysaccharide group. In certain embodiments, the saccharide group comprises a monosaccharide group, a disaccharide group or a trisaccharide group. In certain embodiments, the saccharide group comprises a monosaccharide group and/or a disaccharide group. In certain embodiments, the saccharide group comprises a cyclic form of the saccharide. In certain embodiments, the saccharide group comprises an open chain form of the saccharide.

Examples of monosaccharides comprise a glucose, a fructose, a galactose, a ribose, a mannose and a xylose, a derivative of any of the aforementioned and/or an isomer of any of the aforementioned. Other monosaccharides are contemplated.

Example of disaccharides comprise a sucrose, a maltose, a lactose, a lactulose, a trehalose, a gentiobiose and a cellobiose, a derivative of any of the aforementioned and/or an isomer of any of the aforementioned. Other disaccharides are contemplated.

In certain embodiments, the monosaccharide group comprises a mannose group and/or an isomer thereof. In certain embodiments, the mannose group comprises a mannopyranose group and/or an isomer thereof.

In certain embodiments, the disaccharide group comprises a maltose group and/or an isomer thereof.

In certain embodiments, the saccharide group comprises one or more other groups.

In certain embodiments, the complex comprises the following chemical structure:

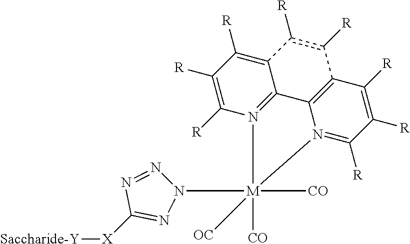

and/or a salt thereof, a solvate thereof, a hydrate thereof, a tautomer thereof or a stereoisomer thereof;
wherein:
M is a transition metal ion;
R is H, hydroxyl, a halogen, optionally substituted alkyl, optionally substituted alkenyl, optionally substituted alkynyl, optionally substituted aryl, optionally substituted heteroalkyl, optionally substituted heterocycle, optionally substituted heteroaryl, optionally substituted alkoxy, optionally substituted aryloxy, optionally substituted heteroaryloxy, optionally substituted arylalkoxy, optionally substituted heteroarylalkoxy, an amine, an amide, a thiol, a phosphorus containing group, or a combination thereof;
X is optionally substituted alkyl, optionally substituted alkenyl, optionally substituted alkynyl, optionally substituted aryl, optionally substituted heteroaryl, optionally substituted aromatic group, optionally substituted aryl or heteroaryl ester, optionally substituted tetrazole, or a combination thereof;
Y is no atom, a linker, NH, NR, O, S, amide, (PO₄⁻), optionally substituted alkyl, optionally substituted alkenyl, optionally substituted alkynyl, optionally substituted aryl, optionally substituted heteroaryl, optionally substituted aromatic group, optionally substituted phenyl, alkyl substituted heteroaryl, optionally substituted aryl or heteroaryl ester, optionally substituted aryl triazole, optionally substituted phenyl triazole, optionally substituted alkyl phenyl triazole or a combination thereof, thereof any of the aforementioned; and
a saccharide comprising one or more of a monosaccharide, a disaccharide and an oligosaccharide.

In certain embodiments, R is a hydrogen group. Other groups are contemplated.

In certain embodiments, M comprises Re(I). Other transition metals are contemplated. Examples of other transition metal ions are described herein and include Iridium (eg Ir(III)) and Ruthenium (eg Ru(II)).

In certain embodiments, X comprises a phenyl or a pyridyl group, and/or a substituted form thereof.

In certain embodiments, Y comprises a triazole. In certain embodiments, Y comprises a 1,2,3 triazole. In certain embodiments, Y comprises an alkyl triazole. In certain embodiments, Y comprises an alkyl 1,2,3 triazole. In certain embodiments, Y comprises a 1-alkyl, 1,2,3 triazole. In certain embodiments, Y comprises a 1-ethyl, 1,2,3 triazole.

In certain embodiments, Y comprises a linker group between the saccharide group and X.

In certain embodiments, the linker comprises NH, NR, O, S, amide, (PO₄⁻), optionally substituted alkyl, optionally substituted alkenyl, optionally substituted alkynyl, optionally substituted aryl, optionally substituted heteroaryl, optionally substituted aromatic group, alkyl substituted heteroaryl, optionally substituted aryl or heteroaryl ester, or a combination thereof, thereof any of the aforementioned.

In certain embodiments, the linker comprises a triazole. In certain embodiments, the linker comprises a 1,2,3 triazole. In certain embodiments, the linker comprises an alkyl triazole. In certain embodiments, the linker comprises an alkyl 1,2,3 triazole. In certain embodiments, the linker comprises a 1-alkyl, 1,2,3 triazole. In certain embodiments, the linker comprises a 1-ethyl, 1,2,3 triazole. In certain embodiments, the linker comprises an alkyl group.

Examples of saccharide groups are described herein. In certain embodiments, the saccharide group comprises one or more of a monosaccharide, a disaccharide, an oligosaccharide or a polysaccharide group. In certain embodiments, the saccharide group is a cyclic form of the saccharide. In certain embodiments, the saccharide group is an open chain form of the saccharide. In certain embodiments, the saccharide group comprises a mannose, a glucose, a galactose, and/or a maltose group.

In certain embodiments, the complex has the following chemical structure:

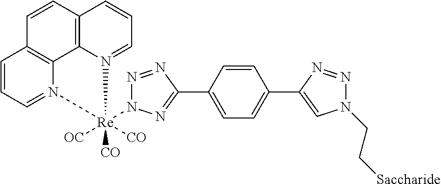

and/or a salt thereof, a solvate thereof, a tautomer thereof, a stereoisomer thereof, or an open chain form of the saccharide;
wherein the saccharide group comprises one or more of a monosaccharide, a disaccharide, an oligosaccharide or a polysaccharide.

In certain embodiments, the complex has the following chemical structure:

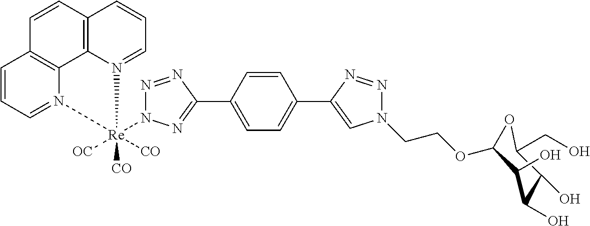

and/or a salt thereof, a solvate thereof, a tautomer thereof, a stereoisomer thereof, a substituted derivative thereof, or an open chain form of the saccharide.

In certain embodiments, the complex has the following chemical structure:

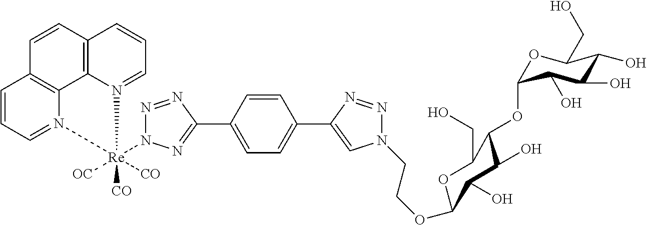

and/or a salt thereof, a solvate thereof, a tautomer thereof, a stereoisomer thereof, a substituted derivative thereof, or an open chain form of the saccharide.

In certain embodiments, the complex has the following chemical structure:

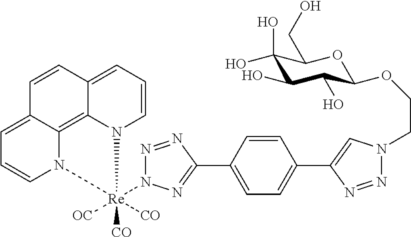

and/or a salt thereof, a solvate thereof, a tautomer thereof, a stereoisomer thereof, a substituted derivative thereof, or an open chain form of the saccharide.

In certain embodiments, the complex has the following chemical structure:

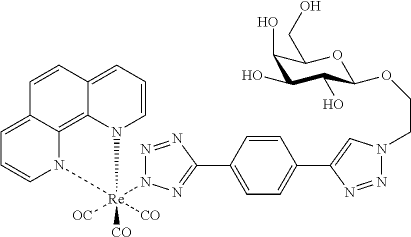

and/or a salt thereof, a solvate thereof, a tautomer thereof, a stereoisomer thereof, a substituted derivative thereof, or an open chain form of the saccharide.

Certain embodiments of the present disclosure provide a complex with one of the following chemical structures:

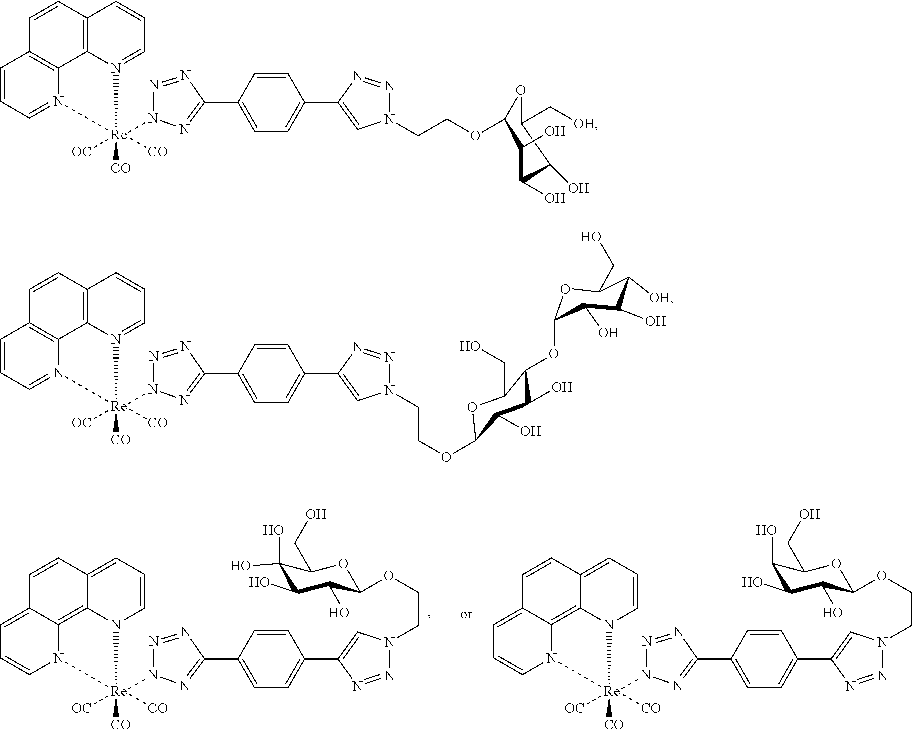

and/or a salt thereof, a solvate thereof, a tautomer thereof, a stereoisomer thereof, a substituted derivative thereof, or an open chain form of the saccharide, of any of the aforementioned chemical structures.

Certain embodiments of the present disclosure provide a complex or compound selected from a complex or compound in the group consisting of complexes or compounds 5 to 8, X1 to X4 or R₀-R₄ in Scheme 1 or Scheme 2, or a complex or compound in the group consisting of complexes or compounds 5, 2, 3 4, X1 to X4, XX2 to XX4, XXX2 to XXX4 in Scheme 3, or Re0 to Re5 in Scheme 4.

Certain embodiments of the present disclosure provide use of a complex, as described herein.

In certain embodiments, the complex is used for imaging of a cell. In certain embodiments, the complex is used for staining a cell. In certain embodiments, the complex is used for intracellular imaging of a cell. In certain embodiments, the complex is used for intracellular imaging of a live cell. In certain embodiments, the complex is used for labelling a cell. In certain embodiments, the complex is used for detecting or labelling a cell structure. In certain embodiments, the complex is used for detecting or labelling an intracellular structure.

In certain embodiments, a complex as described herein is used for diagnosis and/or prognosis. In certain embodiments, a complex as described herein is used as a diagnostic and/or prognostic agent, such as a diagnostic or prognostic agent for prostate cancer.

For example, a complex as described herein may be used to detect prostate cancer, or may be used to detect or identify a pathology associated with aberrant function in muscle cells, such as ischaemia.

Certain embodiments of the present disclosure provide a composition comprising a complex as described herein.

In certain embodiments, the composition is used for imaging of a cell. In certain embodiments, the composition is used for staining of a cell. In certain embodiments, the composition is used for intracellular imaging of a cell. In certain embodiments, the composition is used for intracellular imaging of a live cell. In certain embodiments, the composition is used for labelling a cell. In certain embodiments, the composition is used for detecting or labelling a cell structure. In certain embodiments, the composition is used for detecting or labelling an intracellular structure. In certain embodiment, the composition is used for diagnosis or prognosis.

In certain embodiments, the composition comprises DMSO. Other reagents are contemplated, such as diluents, stabilisers, enhancers, and co-imaging agents.

In certain embodiments, the composition may comprises two or more complexes as described herein, such as for co-imaging, co-labelling or co-staining purposes.

Certain embodiments of the present disclosure provide use of a complex(es) as described herein as an intracellular imaging agent.

Certain embodiments of the present disclosure provide an intracellular imaging agent, the agent comprising a complex as described herein.

Certain embodiments of the present disclosure provide an intracellular imaging agent, the agent comprising a complex comprising a transition metal carbonyl compound, a conjugated bidentate ligand and a tetrazolato compound comprising a saccharide group.

In certain embodiments, the agent consists of a complex as described herein.

In certain embodiments, the agent comprises a derivative of a complex as described herein.

Complexes comprising a transition metal carbonyl compound, a conjugated bidentate ligand and a tetrazolato compound comprising a saccharide group are as described herein.

In certain embodiments, the intracellular imaging agent comprises a complex as described herein coupled to, or associated with, another agent. Methods for coupling are as described herein.

Certain embodiments of the present disclosure provide a composition for intracellular imaging, the composition comprising a transition metal carbonyl compound, a conjugated bidentate ligand and a tetrazolato compound comprising a saccharide group, as described herein.

In certain embodiments, the composition comprises DMSO. Other components are contemplated.

For example, a stock solution of the complex in DMSO may be prepared and the stock solution subsequently diluted in a suitable medium (eg tissue culture medium) for exposing to cells and cell structures.

Certain embodiments of the present disclosure provide a method of intracellular imaging of a cell, the method comprising exposing a cell to a complex as described herein and imaging the complex in the cell.

Certain embodiments of the present disclosure provide a method of intracellular imaging of a cell, the method comprising exposing a cell to a complex comprising a transition metal carbonyl compound, a conjugated bidentate ligand and a tetrazolato compound comprising a saccharide group, and imaging the complex in the cell.

The term “exposing”, and related terms such as “expose” and “exposure”, as used herein refers to contacting and/or treating a species (for example a cell) with an effective amount of a complex (and/or other agent) as described herein. The term includes for example exposing a cell in vitro to a complex as described herein, exposing a cell in vivo to a complex as described herein, exposing a cell ex vivo to a complex as described herein, and administering a complex as described herein to a subject so as to image cells in vivo. Method for exposing a cell to a complex, including administration of agents to a subject, are known in the art. Methods for administering a complex to a subject to image a cell in vivo are known in the art.

In certain embodiments, the cell is a cell from a human, an animal, an insect, a livestock animal (such as a horse, a cow, a sheep, a goat, a pig), a domestic animal (such as a dog or a cat) and other types of animals such as monkeys, rabbits, mice and lab oratory animals.

In certain embodiments, the cell is a cell from a subject suffering from, or susceptible to, a cancer such as prostate cancer.

In certain embodiments, the method comprises exposing the cell to two or more different complexes. For example, a cell may be exposed to two or more different complexes to label/detect/image various types of intracellular structures in the cell.

In certain embodiments, the cell may be a live cell, a fixed cell or a dead cell. In certain embodiments, the cell is a cell for which diagnostic and/or prognostic analysis is to be undertaken. In certain embodiments, the cell is obtained or isolated from a subject for which diagnostic or prognostic testing is to be undertaken. In certain embodiments, the cell is a cancerous cell or a non-cancerous cell.

In certain embodiments, the cell is a live cell.

In certain embodiments, the cell comprises one or more cells in a cell sample, a sample of one or more live cells, one or more fixed cells, one or more dead cells, one or more cells obtained from a subject, a sorted cell, a non-fixed cell, one or more cells in a biologicals sample, one or more cells in a biopsy, one or more cells in a tissue sample, one or more cells in a bodily fluid sample, one or more cells in a blood sample, one or more cells in a urine sample, one or more cells in a saliva sample, one or more cells in a tissue section, one or more cells mounted cells, one or more cells in a tissue sample, and cells generally obtained or isolated from a subject. The cell may be in vitro, ex vivo or in vivo.

In certain embodiments, the cell is present in vivo, in a cell sample, a sample of live cells, a cell extract, a fixed cell, a biopsy, a tissue sample, a bodily fluid sample, a blood sample, a urine sample, or a saliva sample.

In certain embodiments, the cell is a cell in a biological sample. In certain embodiments, the biological sample comprises a cell in vivo, an ex vivo cell and/or a cell in a biological fluid. In certain embodiments, the biological sample comprises a cell in vitro. It will be appreciated that the methods of the present disclosure may be performed in some embodiments wholly in vitro or ex vivo, or wholly in vivo.

Examples of biological samples include a cell sample, a sample of live cells, a sorted cell, a non-fixed cell, a fixed cell, a biopsy, a tissue sample, or a biological fluid (such as blood, plasma, urine, milk, tears, saliva), and/or an extract, component, derivative, processed form or purified form thereof.

It will be appreciated that in some embodiments, the term “cell” as used herein also refers to an extract, lysate, component, derivative, a fixed form, or a processed form of a cell.

Examples of cells are as described herein. In certain embodiments, the cell is a cancerous cell or a non-cancerous cell. In certain embodiments, the cell is a pre-cancerous cell. In certain embodiments, the cell is a tumour cell. In certain embodiments, the cell is a malignant cell.

In certain embodiments, the cell is a myoblast, a muscle cell, a myocyte, or a myocardial cell.

In certain embodiments, the cell is a cancerous prostate cell or a non-cancerous prostate cell.

In certain embodiments, the method is used for imaging of a live cell, in vivo imaging, incorporation into a cell pathway (eg a metabolic pathway, a catabolic pathway), to detect or label a cell, to stain a cell, to detect or label a cellular structure, to detect or label endoplasmic reticulum, to detect or label a vesicular compartment, to detect or label a lysosome, to detect or label cytosol or a cytosolic structure in the cell, to detect or label a macrophage, to detect or label a myocyte, a muscle cell, a myoblast or a myocardial cell, to detect or label a non-cancerous cell and/or a cancerous cell, to identify a non-cancerous cell or a cancerous cell, to screen for cancerous cells, and to distinguish a cancerous cell from a non-cancerous cell. Other uses are contemplated.

In certain embodiments, the cell comprises a live cell.

Certain embodiments of the present disclosure provide a method of labelling a cell.

Certain embodiments of the present disclosure provide a method of labelling a cell, the method comprising exposing the cell to a complex comprising a transition metal carbonyl compound, a conjugated bidentate ligand and a tetrazolato compound comprising a saccharide group, and thereby labelling the cell.

Examples of cells are described herein. In certain embodiments, the cell is a live cell.

Certain embodiments of the present disclosure provide a method of intracellular imaging of a cell.

Certain embodiments of the present disclosure provide a method of intracellular imaging of a live cell by exposing the cell to a complex as described herein. Examples of cells are described herein.

Certain embodiments of the present disclosure provide a method of intracellular imaging of a live cell, the method comprising exposing a live cell to a complex comprising a transition metal carbonyl compound, a conjugated bidentate ligand and a tetrazolato compound comprising a saccharide group, and intracellularly imaging the live cell.

In certain embodiments, the method comprises detecting or labelling a cellular structure. Examples of cellular structures include an endosome, a lysosome, an autophagosome, endoplasmic reticulum, an organelle, Golgi, a vesicle, a compartment, cytosol, a cytosolic structure, a membrane, a plasma membrane or a structure. Other types of cellular structures are contemplated. In certain embodiments, the cellular structure comprises an intracellular structure. In certain embodiments, the cell structure comprises a vesicular structure.

In certain embodiments, the method comprises labelling or detecting a vesicular structure in a cell.

In certain embodiments, the method comprises labelling or detecting a vesicular structure and the complex has the following chemical structure:

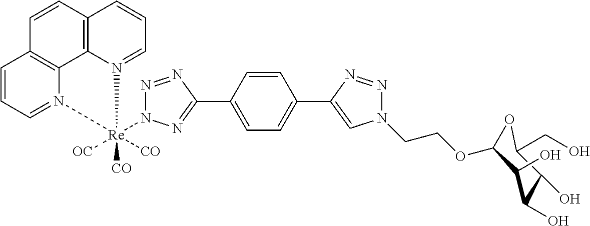

and/or a salt thereof, a solvate thereof, a tautomer thereof, a stereoisomer thereof, a substituted derivative thereof, or an open chain form of the saccharide.

Certain embodiments of the present disclosure provide a method of labelling or detecting a vesicular structure in a cell, the method comprising exposing the cell to a complex with the following chemical structure:

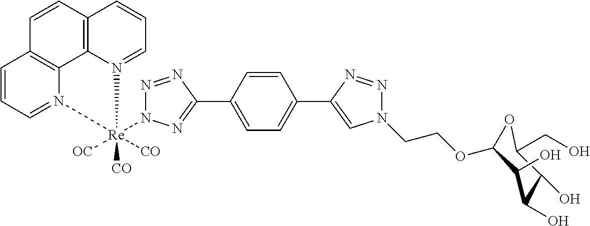

and/or a salt thereof, a solvate thereof, a tautomer thereof, a stereoisomer thereof, a substituted derivative thereof, or an open chain form of the saccharide, and thereby labelling or detecting a vesicular structure in the cell.

In certain embodiments, the method excludes labelling of lipid droplets in the cell.

In certain embodiments, the vesicular structure comprises a lysosome.

Certain embodiments of the present disclosure provide a method of labelling or detecting endoplasmic reticulum and/or a lysosome in a cell, the method comprising exposing the cell to a complex with the following chemical structure:

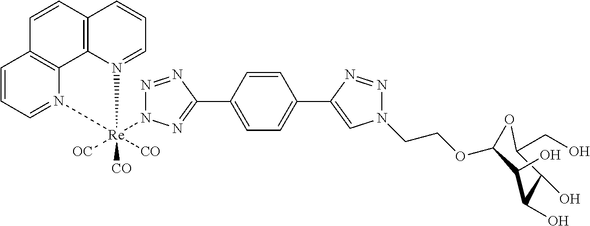

and/or a salt thereof, a solvate thereof, a tautomer thereof, a stereoisomer thereof, a substituted derivative thereof, or an open chain form of the saccharide, and labelling or detecting the endoplasmic reticulum and/or the lysosome in the cell.

In certain embodiments, the cellular structure comprises cytosol or a cytosolic structure and the and the complex has the following chemical structure:

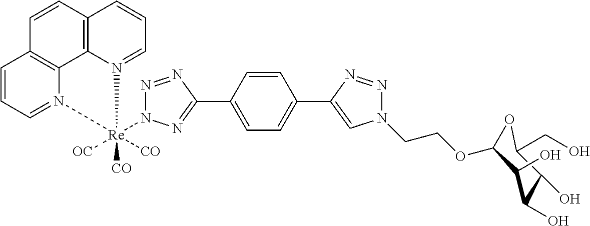

and/or a salt thereof, a solvate thereof, a tautomer thereof, a stereoisomer thereof, a substituted derivative thereof, or an open chain form of the saccharide.

In certain embodiments, the method comprises labelling or detecting cytosol or a cytosolic structure and the complex has the following chemical structure:

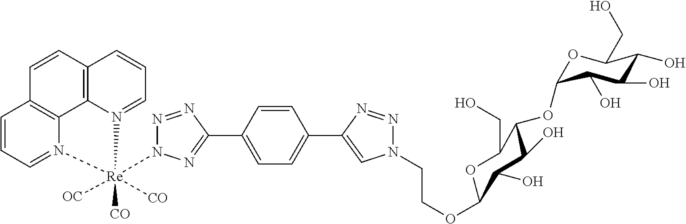

and/or a salt thereof, a solvate thereof, a tautomer thereof, a stereoisomer thereof, a substituted derivative thereof, or an open chain form of the saccharide.

Certain embodiments of the present disclosure provide a method of labelling or detecting cytosol or a cytosolic structure in a cell, the method comprising exposing a cell to a complex with the following chemical structure:

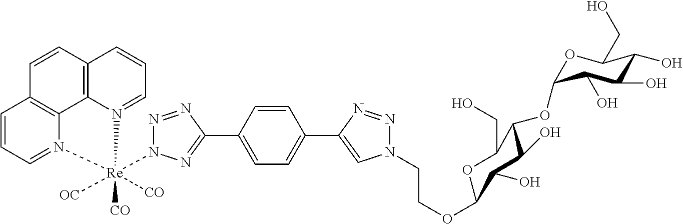

and/or a salt thereof, a solvate thereof, a tautomer thereof, a stereoisomer thereof, a substituted derivative thereof, or an open chain form of the saccharide, and thereby labelling or detecting cytosol and/or a cytosolic structure in the cell.

In certain embodiments, the method excludes labelling of lipid droplets in the cell.

Certain embodiments of the present disclosure provide a method of labelling or detecting a cellular structure using a complex as described herein.

Certain embodiments of the present disclosure provide a method of identifying a cellular structure using a complex as described herein.

In certain embodiments, the cellular structure comprises an intracellular structure. In certain embodiments, the cellular structure comprises a subcellular structure and/or a cellular compartment. Examples of cellular structures include a subcellular structure, a cellular compartment, endosomes, lysosomes and/or autophagosomes, endoplasmic reticulum, cytosol, a cytosolic structure (such as glycogen), an organelle, Golgi, a membrane, a plasma membrane, a vesicle, or lipid droplets.

Certain embodiments of the present disclosure provide a method of identifying a cancer cell. In certain embodiments, the cancer cell is a prostate cancer cell.

In certain embodiments, the cancerous cell is identified or distinguished from a non-cancerous cell by differential labelling of the cancerous cell with the complex. For example, the differential labelling may be altered labelling of the cell, altered localisation of the complex in the cell and/or altered distribution in the cell. The differential labelling of the cell may, for example, be as compared to a non-cancerous cell, a cancerous cell, the labelling of a reference cell (eg a cancer cell), or one or more characteristics associated with a non-cancerous cell or a cancerous cell. Other methods for assessing differential labelling are contemplated.

Certain embodiments of the present disclosure provide a method of identifying a cancerous cell, the method comprising exposing the cell to a complex comprising a transition metal carbonyl compound, a conjugated bidentate ligand and a tetrazolato compound comprising a saccharide group, and identifying the cell as a cancerous cell on the basis of differential labelling of the cell with the complex.

Certain embodiments of the present disclosure provide a method of identifying a cancerous cell, the method comprising exposing the cell to a complex comprising a transition metal carbonyl compound, a conjugated bidentate ligand and a tetrazolato compound comprising a saccharide group, and identifying the cell as a cancerous cell on the basis of altered labelling of the cell with the complex and/or altered localisation of the complex in the cell.

In certain embodiments, a complex as described herein is used in conjunction with one or more other agents, markers, stains, or labels to identify or screen for a cancerous cell.

In certain embodiments, the cancerous cell is a cancerous prostate cell and the method comprises exposing the cell to a complex with the following chemical structure:

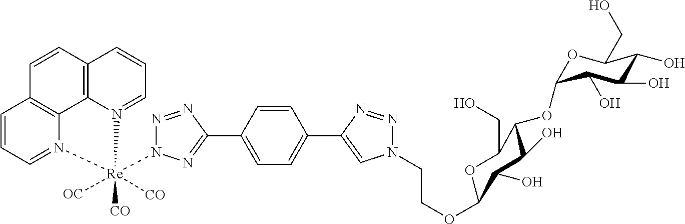

and/or a salt thereof, a solvate thereof, a tautomer thereof, a stereoisomer thereof, a substituted derivative thereof, or an open chain form of the saccharide.

Certain embodiments of the present disclosure provide a method of identifying a cancerous prostate cell, the method comprising exposing the cell to a complex with the following chemical structure:

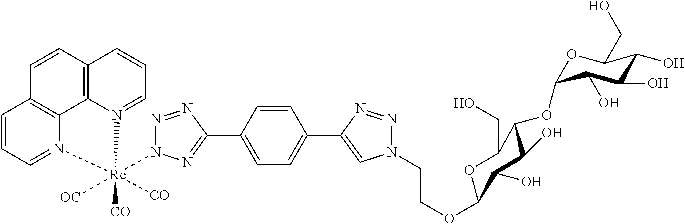

and/or a salt thereof, a solvate thereof, a tautomer thereof, a stereoisomer thereof, a substituted derivative thereof, or an open chain form of the saccharide, and identifying the cell as a cancerous prostate cell on the basis of altered labelling of the cell with the complex and/or altered localisation of the complex in the cell.

In certain embodiments, the method comprises identifying a cancerous prostate cell on the basis of increased labelling of the cell with the complex and/or altered localisation or distribution of the complex in the cell.

In certain embodiments, one or more of cytoplasmic labelling of the cell with the complex, localisation of the complex in punctate structures in the cytosol of the cell and increased perinuclear localisation of the complex in the cell are indicative of a cancerous prostate cell.

Certain embodiments of the present disclosure provide a method of screening for a cancerous cell, the method comprising exposing a cell to a complex as described herein and identifying the cell as a cancerous cell on the basis of one or more of altered labelling of the cell with the complex and/or altered localisation of the complex in the cell.

In certain embodiments, the cancerous cell is a cancerous prostate cell and the method comprises exposing the cell to a complex with the following chemical structure:

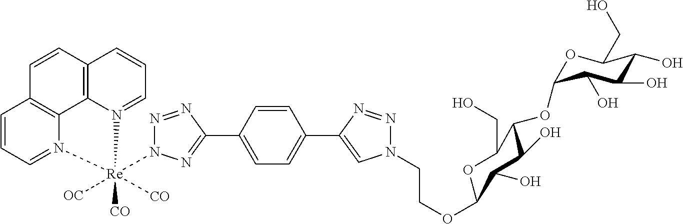

and/or a salt thereof, a solvate thereof, a tautomer thereof, a stereoisomer thereof, a substituted derivative thereof, or an open chain form of the saccharide.

In certain embodiments, the method comprises identifying a cancerous prostate cell on the basis of increased labelling of the cell with the complex and/or altered localisation or distribution of the complex in the cell.

In certain embodiments, one or more of cytoplasmic labelling of the cell with the complex, localisation of the complex in punctate structures in the cytosol of the cell and increased perinuclear localisation of the complex in the cell are indicative of a cancerous prostate cell.

Certain embodiments of the present disclosure provide a method of screening for a cancerous prostate cell, the method comprising exposing the cell to a complex with the following chemical structure:

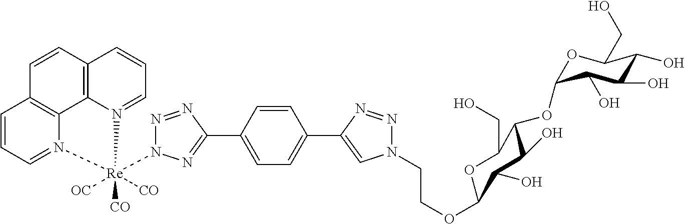

and/or a salt thereof, a solvate thereof, a tautomer thereof, a stereoisomer thereof, a substituted derivative thereof, or an open chain form of the saccharide, and identifying the cell as a cancerous prostate cell on the basis of one or more of increased labelling of the cell with the complex, cytoplasmic labelling of the cell with the complex, localisation of the complex in punctate structures in the cytosol of the cell and increased perinuclear localisation of the complex in the cell.

Certain embodiments of the present disclosure provide a method of identifying a prostate cancer cell.

Certain embodiments of the present disclosure provide a method of identifying prostate cancer in a subject.

Subjects are as described herein. Examples of subjects include humans, animals, such as livestock animals (eg a horse, a cow, a sheep, a goat, a pig), a domestic animal (eg a dog or a cat) and other types of animals such as monkeys, rabbits, mice and laboratory animals, and insects. Other types of subjects are contemplated.

In certain embodiments, the method comprises obtaining one or more cells from the subject and exposing the cells so obtained to the complex. In certain embodiments, the method comprises exposing cells isolated from a subject to the complex. In certain embodiments, the method comprises obtaining a biological sample from a subject and exposing cells in the sample to the complex. Examples of biological samples are described herein. In certain embodiments, the method comprises exposing cells isolated from a subject to the complex. Methods for obtaining cells are known in the art. For example, one or more cells may be obtained by taking a biopsy, a tissue sample or a blood sample from the subject, and cells labelled with a complex as described herein.

In certain embodiments, the method comprises intracellular imaging. In certain embodiments, the method comprises intracellular imaging of cells. In certain embodiments, the intracellular imaging comprises intracellular imaging of live cells.

In certain embodiments, the method comprises intracellular imaging in vivo. In certain embodiments, the method comprises intracellular imaging ex vivo. In certain embodiments, the method comprises intracellular imaging in vitro.

In certain embodiments, the method is used to detect the presence of cancerous prostate cells, or a prostate cancer, in a subject. In certain embodiments, the method is used to detect the presence or absence of a prostate cancer in a subject. In certain embodiments, the method is used to screen for the presence or absence of a prostate cancer in a subject. In certain embodiments, the method is used to exclude the presence a prostate cancer in a subject.

Certain embodiments of the present disclosure provide use of a complex as described herein to identify a subject suffering from, or susceptible to, a cancer.

Certain embodiments of the present disclosure provide use of a complex as described herein to identify a subject suffering from, or susceptible to, prostate cancer.

Certain embodiments of the present disclosure provide a method of identifying a subject suffering from, or susceptible to, prostate cancer.

Certain embodiments of the present disclosure provide a method of identifying a cancer in a subject, the method comprising exposing a cell from the subject to a complex as described herein and identifying cancer in the subject on the basis of one or more of altered labelling of the cell with the complex and/or altered localisation of the complex in the cell.

In certain embodiments, the cancer is a prostate cancer and the method comprises exposing a cell from the subject to a complex with the following chemical structure:

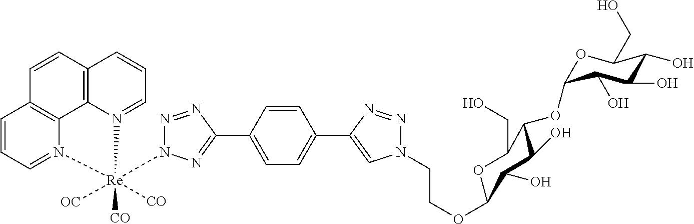

and/or a salt thereof, a solvate thereof, a tautomer thereof, a stereoisomer thereof, or an open chain form of the saccharide.

In certain embodiments, the method comprises identifying prostate cancer on the basis of increased labelling of the cell with the complex and/or altered localisation or distribution of the complex in the cell.

In certain embodiments, one or more of cytoplasmic labelling of the cell with the complex, localisation of the complex in punctate structures in the cytosol of the cell and increased perinuclear localisation of the complex in the cell is indicative of prostate cancer.

Certain embodiments of the present disclosure provide a method of identifying a prostate cancer in a subject, the method comprising exposing a cell from the subject to a complex with the following chemical structure:

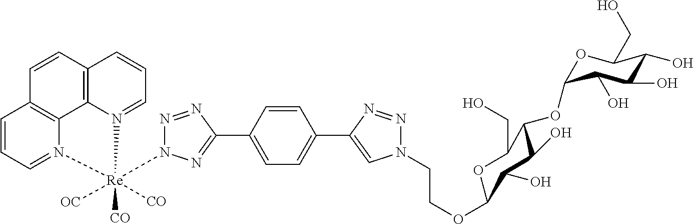

and/or a salt thereof, a solvate thereof, a tautomer thereof, a stereoisomer thereof, a substituted derivative thereof, or an open chain form of the saccharide, and identifying prostate cancer in the subject on the basis of altered labelling of the cell with the complex and/or altered localisation of the complex in the cell.

Certain embodiments of the present disclosure provide a method of identifying a prostate cancer in a subject, the method comprising exposing the subject to a complex with the following chemical structure:

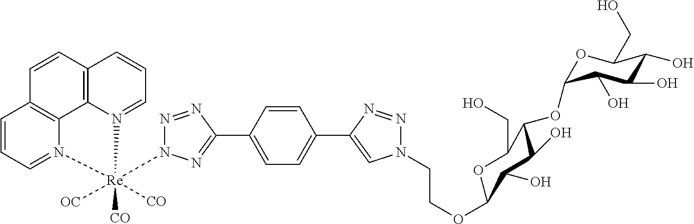

and/or a salt thereof, a solvate thereof, a tautomer thereof, a stereoisomer thereof, a substituted derivative thereof, or an open chain form of the saccharide, and identifying prostate cancer in the subject on the basis of altered labelling of prostate cells with the complex and/or altered localisation of the complex in prostate cells.

Certain embodiments of the present disclosure provide a method of identifying a subject with an increased likelihood or risk of cancer.

In certain embodiments, the cancer is a prostate cancer.

Certain embodiments of the present disclosure provide a method of identifying a subject with an increased likelihood or risk of a prostate cancer, the method comprising exposing the subject to a complex with the following chemical structure:

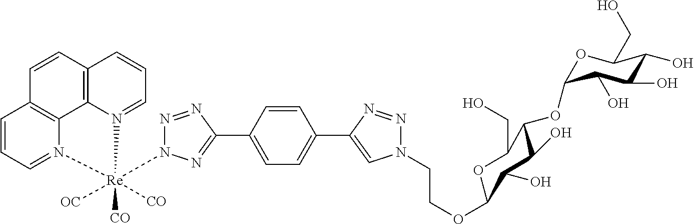

and/or a salt thereof, a solvate thereof, a tautomer thereof, a stereoisomer thereof, a substituted derivative thereof, or an open chain form of the saccharide, and identifying the subject as having an increased likelihood or risk of prostate on the basis of altered labelling of prostate cells with the complex and/or altered localisation of the complex in prostate cells.

In certain embodiments, an increased likelihood or risk of prostate cancer is made on the basis of one or more of increased labelling of the cell with the complex, cytoplasmic labelling of the cell with the complex, localisation of the complex in punctate structures in the cytosol of the cell and increased perinuclear localisation of the complex in the cell.

Certain embodiments of the present disclosure provide a method of identifying a diagnostic or a prognostic marker using a complex as described herein.

In certain embodiments, the diagnostic or prognostic marker is associated with the presence of absence of a cancer.

Certain embodiments of the present disclosure provide a method of identifying a diagnostic or prognostic marker associated with the presence or absence of a cancer, the method comprising using a complex comprising a transition metal carbonyl compound, a conjugated bidentate ligand and a tetrazolato compound comprising a saccharide group to identify a marker associated with the presence or absence of the cancer.

In certain embodiments, the cancer is a prostate cancer.

Certain embodiments of the present disclosure provide a method of identifying a diagnostic or prognostic marker associated with the presence or absence of prostate cancer, the method comprising using a complex comprising a transition metal carbonyl compound, a conjugated bidentate ligand and a tetrazolato compound comprising a saccharide group to identify a marker associated with the presence or absence of prostate cancer.

Certain embodiments of the present disclosure provide a method of identifying a diagnostic or prognostic marker associated with the likelihood or risk of a prostate cancer, the method comprising using a complex comprising a transition metal carbonyl compound, a conjugated bidentate ligand and a tetrazolato compound comprising a saccharide group to identify a marker associated with the likelihood or risk of a prostate cancer.

Methods for identifying markers are known in the art. Methods for using complexes are as described herein.

Certain embodiments of the present disclosure provide a kit.

In certain embodiments, the kit comprises one or more reagents and/or instructions as described herein, including one or more complexes as described herein.

For example, the kit may also include instructions for using a complex as describe herein, instructions for exposing cells to the complex and/or instructions for imaging or labelling cells.

Certain embodiments of the present disclosure provide a kit for performing a method as described herein.

Certain embodiments of the present disclosure provide a kit for intracellular imaging of cells, the kit comprising a complex comprising a transition metal carbonyl compound, a conjugated bidentate ligand and a tetrazolato compound comprising a saccharide group.

The kit may also include one or more other reagents/components as described herein, instructions for using the complex, instructions for exposing the cells to the complex and/or instructions for imaging the cells.

In certain embodiments, the kit further comprises one or more other reagents for imaging or labelling of cells, including enhancers, stabilisers and controls.

In certain embodiments, the kit comprises DMSO and/or the complex in DMSO.

In certain embodiments, the kit is a kit for intracellular imaging of live cells.

Certain embodiments of the present disclosure provide a kit for intracellular imaging of live cells, the kit comprising a complex comprising a transition metal carbonyl compound, a conjugated bidentate ligand and a tetrazolato compound.

In certain embodiments, the kit further comprises one or more other reagents for imaging of live cells, including enhancers, stabilisers and controls.

In certain embodiments, the kit comprises DMSO, and/or the complex in DMSO and/or the complex in DMSO being further diluted into another medium, such as tissue culture medium.

Certain embodiments of the present disclosure provide a kit for labelling or detecting an intracellular structure, the kit comprising a complex comprising a transition metal carbonyl compound, a conjugated bidentate ligand and a tetrazolato compound comprising a saccharide.

In certain embodiments, the kit further comprises instructions. For example, the kit may also include instructions for labelling or detecting intracellular structures, instructions for exposing intracellular structures and/or cells to the complex, and/or instructions for detecting and/or visualising the complex.

In certain embodiments, the kit further comprises one or more other reagents for labelling or detection, including enhancers, stabilisers and controls.

In certain embodiments, the kit comprises DMSO and/or complex in DMSO.

Certain embodiments of the present disclosure provide a kit comprising one or more of (i) a complex as described herein, (ii) a composition as described herein, (iii) an intracellular imaging agent as described herein, or (iv) for performing a method as described herein.

Certain embodiments of the present disclosure provide a method using a kit as described herein.

Certain embodiments of the present disclosure provide a method for identifying a complex suitable for imaging or labelling a cell.

Certain embodiments of the present disclosure provide a method for identifying intracellular imaging agents.

Certain embodiments of the present disclosure provide screening methods for identifying a complex for intracellularly imaging a cell.

Such methods may be used to screen new reagents for research and diagnostic/prognostic purposes. For example, complexes so identified may have utility for diagnostic purposes for prostate cancer.

Methods for assessing the ability of a complex to intracellular image/label a cell are described herein.

Candidate complexes comprising a transition metal carbonyl compound, a conjugated bidentate ligand and a tetrazolato compound comprising a saccharide group are as described herein. Determination of the ability to intracellular image a cell may be performed in vitro, in vivo, in an animal or insect model, for example. Intracellular imaging agents may be identified on the basis that the candidate complex intracellularly labels cells.

Certain embodiments of the present disclosure provide a method of identifying a complex for intracellular imaging of a cell, the method comprising:
providing a candidate complex comprising a complex comprising a transition metal carbonyl compound, a conjugated bidentate ligand and a tetrazolato compound comprising a saccharide group;
determining the ability of the candidate complex to intracellularly label a cell; and
identifying the candidate complex as a complex for intracellular imaging of a cell.

Certain embodiments of the present disclosure provide a method of identifying an intracellular imaging agent, the method comprising:
providing a candidate complex comprising a complex comprising a transition metal carbonyl compound, a conjugated bidentate ligand and a tetrazolato compound comprising a saccharide group;
determining the ability of the candidate complex to intracellularly label a cell; and
identifying the candidate complex as an intracellular imaging agent.

Certain embodiments of the present disclosure provide a complex for intracellular imaging, or an intracellular imaging agent, identified by a method as described herein.

Certain embodiments of the present disclosure provide a method of synthesizing or producing a complex comprising a transition metal carbonyl compound, a conjugated bidentate ligand and a tetrazolato compound comprising a saccharide group.

Certain embodiments of the present disclosure provide a method of producing a complex comprising a transition metal carbonyl compound, a conjugated bidentate ligand and a tetrazolato compound comprising a saccharide group, the method comprising:
providing a complex comprising a transition metal carbonyl compound, a conjugated bidentate ligand and a tetrazolato compound and reacting the tetrazolato compound with a saccharide and/or a derivative thereof; or
providing a transition metal carbonyl compound, a conjugated bidentate ligand and a tetrazolato compound comprising a saccharide group and forming a complex from the aforementioned components; and
producing a complex comprising a transition metal carbonyl compound, a conjugated bidentate ligand and a tetrazolato compound comprising a saccharide group.

Reaction methods are as described herein.

In certain embodiments, the method comprises reacting a saccharide with an attached azide group with a complex comprising a transition metal carbonyl compound, a conjugated bidentate ligand and a tetrazolato compound comprising an attached alkynyl group. For example, a ethynyl phenyltetrazolate may be reacted with an ethyl azido saccharide.

In certain embodiments, the method comprises reacting a saccharide with an attached alkynyl group with a complex comprising a transition metal carbonyl compound, a conjugated bidentate ligand and a tetrazolato compound comprising an azide group. For example, an azido phenyltetrazolate may be reacted with an ethynyl ethyl saccharide.

In certain embodiments, the complex is formed by providing a transition metal carbonyl compound, a conjugated bidentate ligand and a tetrazolato compound comprising a saccharide group and forming a complex from the aforementioned components. In certain embodiments, the complex is formed sequentially from one or more of the components, in any suitable order. In certain embodiments, the complex is not formed sequentially from one or more of the components.

In certain embodiments, the complex is formed from a combination of the one or more of the components complexed together and forming a final complex therefrom from any other of the remaining components. For example, a complex comprising a complex comprising a transition metal carbonyl compound, a conjugated bidentate ligand may be exposed to a tetrazolato compound comprising a saccharide group to form the complex. Alternatively, a transition metal carbonyl compound may be exposed to a conjugated bidentate ligand and then subsequently exposed to a tetrazolato compound comprising a saccharide group.

Certain embodiments of the present disclosure provide a complex produced by a method as described herein.

Certain embodiments of the present disclosure provide a complex with the following formula:

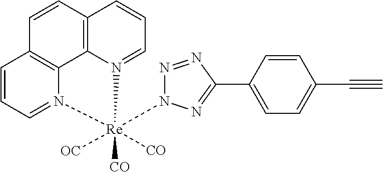

or salt thereof, a solvate thereof, a stereoisomer thereof, or a substituted derivative thereof.

Certain embodiments of the present disclosure provide use of such a complex for synthesising or producing a complex comprising a Re(I) tricarbonyl compound, a conjugated 1, 10 phenanthroline ligand and a pheny tetrazolato compound comprising a saccharide group.

Certain embodiments of the present disclosure provide method of coupling an agent to a complex comprising a transition metal carbonyl compound, a conjugated bidentate ligand and a tetrazolato compound, the method comprising using a complex comprising a transition metal carbonyl compound, a conjugated bidentate ligand and an alkyne tetrazolato compound to couple the agent to the complex.

Certain embodiments of the present disclosure provide use of a Re(I) tricarbonyl compound, a conjugated 1, 10 phenanthroline ligand and an alkynyl tetrazolato compound to couple an agent to the complex.

Certain embodiments of the present disclosure provide method of coupling an agent to a complex comprising a transition metal carbonyl compound, a conjugated bidentate ligand and a tetrazolato compound, the method comprising using a complex comprising a Re(I) tricarbonyl compound, a conjugated 1, 10 phenanthroline ligand and an alkynyl tetrazolato compound to couple the agent to the complex.

In certain embodiments, the method is used to couple an agent comprising a saccharide, a polypeptide, or a nucleic acid to the complex. Other types of agents are contemplated.

In certain embodiments, the method is used to fluorescently label a saccharide, a polypeptide, or a nucleic acid to the complex. Other types of agents for labelling are contemplated.

The present disclosure is further described by the following examples. It is to be understood that the following description is for the purpose of describing particular embodiments only and is not intended to be limiting with respect to the above description.

### Example 1—Synthesis, Characterisation and Cellular Uptake of Saccharide-Targeted, Mononuclear Tricarbonyl Rhenium(I) Tetrazolato Complexes

1. Synthesis of Targeted Re(I) Tetrazolato Complexes

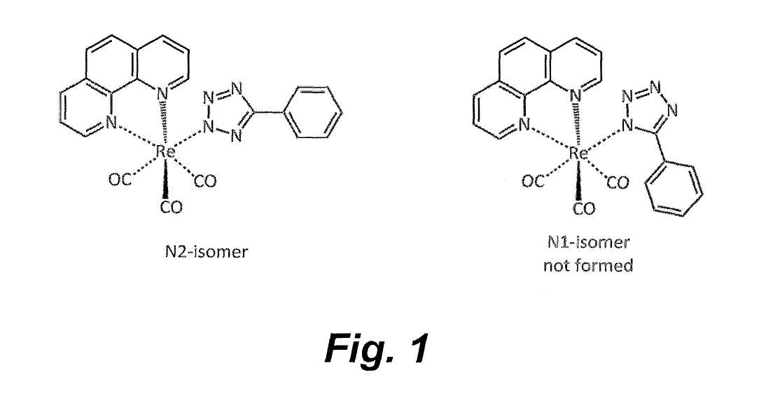

Complexes of the type fac-[Re(CO)₃(diim)(L)]^{0/+} are often synthesised from a neutral parent complex where the ancillary ligand (L) is a halogen, such as chlorine or bromine. Replacing this ligand with a ligand of the phenyltetrazolate type produces improved photophysical properties owing to the σ donating and π accepting nature of the ligand. 2 These improved characteristics are a direct result of metal-to-ligand backbonding between Re(I) and the phenyltetrazolate ligand, which widens the HOMO-LUMO gap and decreases the non-radiative decay mechanism of the complex in accordance with the energy gap law. Although there are two possible linkage isomers for phenyltetrazolate ligands, investigation has shown that only the N2 coordinated linkage isomer is formed (FIG. 1).

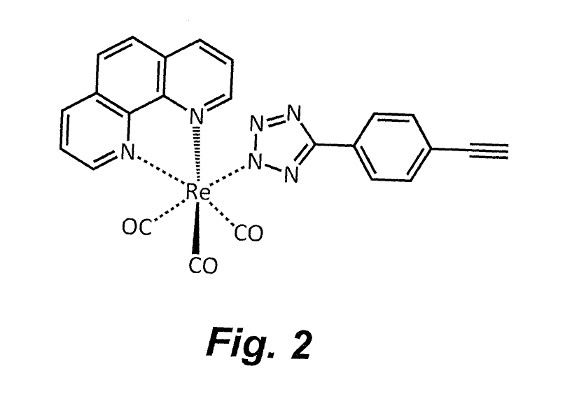

The photophysical properties of a Re(I) complex can be tuned by attachment of substituents at the meta or para position of the phenyltetrazolate ligand. The photophysical properties of Re(I) tetrazolato complexes are ideal for cellular imaging applications, though a lack of water solubility appears to be a limiting factor. Hence, modification of the parent complex to enhance water solubility would enable a Re(I) complex to be employed as a cellular imaging agent. The attachment of a saccharide residue to a Re(I) complex will not only enhance water solubility of the complex, but will also allow potential targeted uptake via, for example, a saccharide receptor. In order to attach a substituent, a Re(I) complex can be designed with alkyne functionality at the para position of the phenyltetrazolate ligand (FIG. 2). This alkyne may then be reacted with a corresponding azido compound.

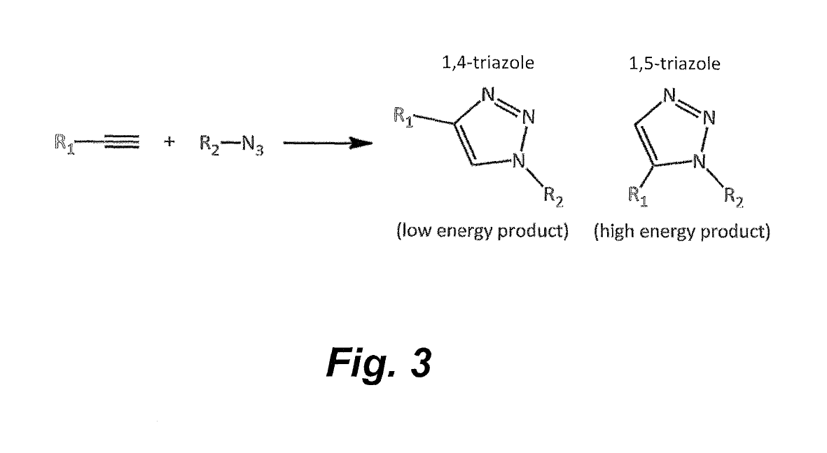

One type of chemistry is the 1,3-dipolar cycloaddition between an alkyne and an azide, forming a 1,4- or 1,5-triazole link (FIG. 3).

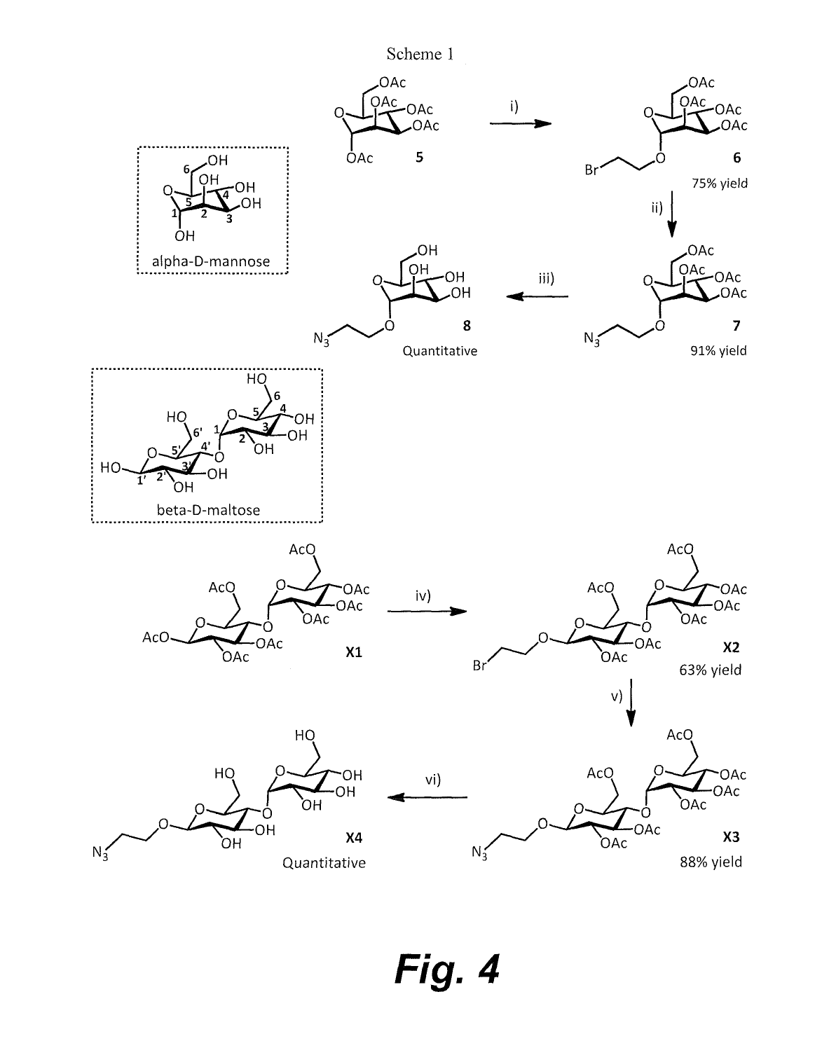

The regioselectivity of the reaction between an azide and an alkyne can be controlled using copper(I) as a catalyst ensuring the formation of the low energy 1,4-triazole linker in a fast, high yielding, one step reaction. In order to attach the saccharide residues to a Rhenium(I) complex, they were synthetically modified to include an azido functional group as detailed in Scheme 1, shown in FIG. 4.

Reagents and conditions for Scheme 1: i) 2-Bromoethanol/BF₃Et₂O/DCM/24 h/0→25° C.; ii) NaN₃/DMF/100° C./1.5 h; iii) NaOMe/MeOH/24 h; iv) 2-Bromoethanol/BF₃Et₂O/DCM/24 h/0→25° C.; v) NaN₃/DMF/100° C./1.5 h; vi) NaOMe/MeOH/24 h.

2-azidoethoxy-α-D-mannose (8) was synthesised as follows: α-D-mannose pentaacetate (5) was alkylated with 2-bromoethanol (6), converted to an azide (7) with NaN₃, then deprotected using NaOMe to obtain 2-azidoethoxy-α-D-mannose (8) with an overall yield of 68%. Compound 5 was alkylated with 2-bromoethanol in the presence of boron trifluorodiethyletherate (BF3Et₂O) at 0° C. to produce a thick syrup. The syrup was then crystallised from ether/DCM and the corresponding brominated sugar (6) was isolated as white crystals in 75% yield. Successful substitution of the C1-acetyl group was confirmed by NMR spectroscopy, which showed a shift in the position of the C1 proton resonance from 6.00 to 4.88 ppm, corresponding with the appearance of a multiplet at 3.87 ppm (OCH₂) and triplet at 3.52 ppm (BrCH₂) in the ¹H NMR spectrum. ¹H and ¹³C NMR spectra both showed only 4 acetate resonances (COCH₃; 2.00, 2.06, 2.11, 2.16 ppm and COCH₃; 20.6, 20.7, 20.8, 20.9 ppm) consistent with a 2,3,4,6-acetylated mannose residue. Electrospray ionisation mass spectrometry (ESI-MS) detected a molecular ion at m/z=455/457 (Bromine isotopes) with corresponding sodium adducts at m/z=478/480, confirming the molecular weight expected after successful alkylation.

Compound 6 was then treated with 6 equivalents of sodium azide (NaN₃) in dimethylformamide (DMF) at 100° C. for 1 h, yielding the protected 2-azidoethyl-2,3,4,6-tetra-O-acetyl-α-D-mannopyranose residue (7) as a thin, colourless syrup. Upon scratching, the syrup crystallised to form transparent white crystals in 91% yield. When scaling the reaction up to quantities over 200 mg, the heating time was increased to 1.5 hrs to ensure the reaction went to completion. Replacement of the alkyl halide was confirmed by ¹H NMR and ¹³C NMR spectroscopy, which both showed a downfield shift in the terminal CH₂ resonances of the C1-alkyl chain corresponding to the electron withdrawing nature of the azido (—N₃) functional group. ESI-MS detected a molecular ion at m/z=418 with the corresponding sodium adduct at m/z=441.

The corresponding deprotection of compound 7 was using NaOMe (6 equivalents). The solvent was evaporated and dry MeOH was used to extract the product from the dry solid residue. The process of extracting with dry MeOH, then filtering through a 0.45 μm nylon syringe filter, was repeated until all of the excess salt was removed. Successful deprotection was confirmed by the disappearance of the acetate resonances in the ¹H and ¹³C NMR spectra, and the condensation of the C1-6 proton resonances into the range of 3.36-3.99 ppm. ESI-MS detected a molecular ion at m/z=250.1 with corresponding sodium adduct at m/z=273.0. For each mannose residue (6, 7 and 8), the C1-H resonance was observed as a singlet in the range of 4.88-4.93, confirming the presence of only one isomer (a).

Following a similar synthetic pathway, β-D-maltose octaacetate (X1) was alkylated with 2-bromoethanol in the presence of boron trifluorodiethyletherate (BF₃Et₂O) at 0° C. to yield a viscous syrup. The syrup was analysed by NMR spectroscopy and showed the presence of multiple products. The mixture was then purified by silica gel column chromatography eluting the product with 30:70 ethyl acetate/hexane. The product (X2) was isolated as a white solid in 63% yield. Analysis by TLC revealed one spot with Rf=0.41. Successful substitution of the C1′-acetyl group (see Scheme 1 insert for β-D-maltose numbering) was confirmed by NMR spectroscopy, which showed a shift in the position of the C1′ proton resonance from 5.66 to 4.55 ppm, corresponding with the appearance of 2 multiplets at 3.76 and 4.07 ppm (OCH₂) and a multiplet at 3.37 ppm (BrCH₂) in the 1H NMR spectrum. ¹H NMR spectra showed 6 acetate resonances corresponding to 21 protons (COCH₃; 1.97, 1.97, 1.99, 2.01, 2.07, 2.11 ppm and ¹³C NMR spectra showed 7 acetate resonances (COCH3; 20.7, 20.8, 20.9, 20.9, 21.0, 21.1, 21.2 ppm), which were both consistent with a C1′ substituted acetylated maltose residue. Electrospray ionisation mass spectrometry (ESI-MS) detected a molecular ion at m/z=761/763 (M+H₃O⁺)(Bromine isotopes), confirming the molecular weight expected after successful alkylation with 2-bromoethanol.

Compound X2 was then treated with 6 equivalents of sodium azide (NaN₃) in dimethylformamide (DMF) at 100° C. for 1.5 h, which afforded the protected 2-azidoethyl-2′,3′,6′,2″,3″,4″,6″-hepta-O-acetyl-β-D-maltopyranose residue (X3) as a thin, colourless syrup. Upon scratching, the syrup crystallised to form transparent white crystals in 88% yield. Replacement of the alkyl halide was confirmed by ¹H NMR and ¹³C NMR spectroscopy, which both showed a downfield shift in the terminal CH₂ resonances of the C1-alkyl chain corresponding to the electron withdrawing nature of the azido (—N₃) functional group. ESI-MS detected a molecular ion at m/z=723 which corresponded with the molecular ion plus water.

The deprotection of X3 was performed using 5 equivalents of sodium methoxide (NaOMe) in dry MeOH. The solvent was evaporated and dry MeOH was used to extract the product from the dry solid residue. The MeOH extract was then filtered through a 0.45 μm nylon syringe filter, to remove excess salt. This process was repeated with dry EtOH to ensure all of the excess salt was removed. The lack of salt was confirmed by comparison of the mass of the expected product with the mass of the actual product, as the reaction is expected to proceed in quantitative yield. The product 2-azidoethyl-β-D-maltose (X4) was afforded as a white solid in quantitative yield. The identity of the product was confirmed by the disappearance of the acetate resonances in the ¹H and ¹³C NMR spectra, and the condensation of the C2-6 proton resonances into the range of 3.58-4.09 ppm. ESI-MS detected a molecular ion at m/z=434 corresponding to the molecular ion plus sodium. Analysis by NMR showed that for each maltose residue (X1, X2, X3 and X4), the C1′-H resonance was observed as a doublet, as expected, confirming the presence of only one isomer (β) throughout the synthesis.

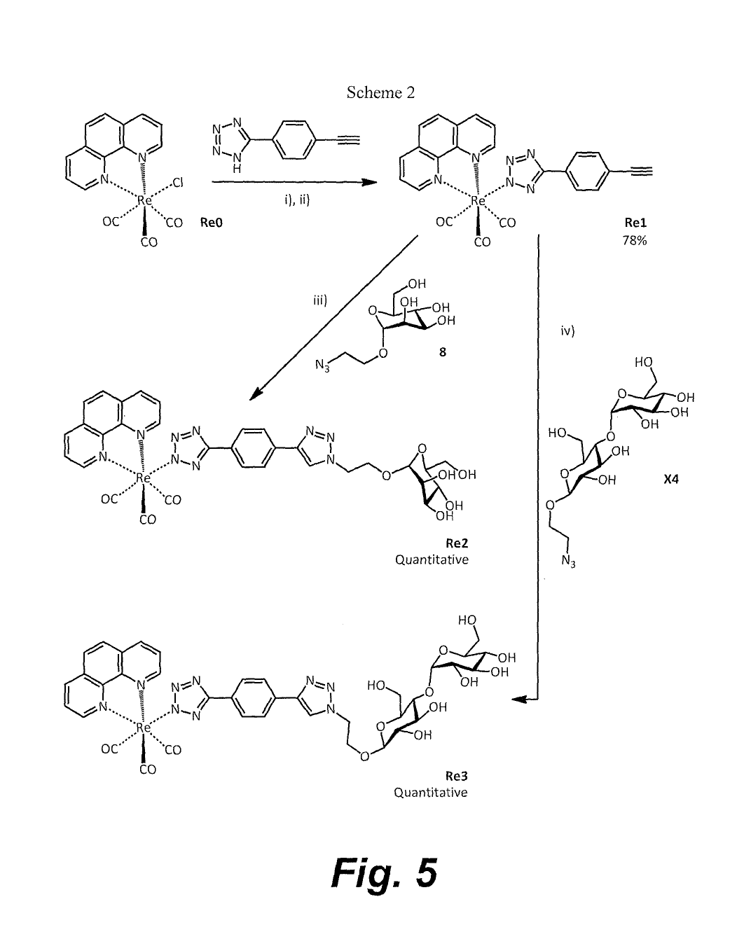

Coordination of an azide functionalised mannose or maltose residue to a tricarbonyl phenanthroline (4-ethynylphenyltetrazolato) Rhenium(I) framework, was achieved using the reaction detailed in Scheme 2 as shown in FIG. 5.

Scheme 2: Synthesis of targeted Rhenium tetrazolato complexes. Reagents and conditions: i) AgBF₄/ CH₃CN/reflux/dark/4 h; ii) 5-(4-ethynylphenyl)-1H tetrazole/NEt₃/CH₃CN/reflux/24 h; iii) CuSO₄.5H2O/Na.Asc/DMF/H₂O/24 h; iv) CuSO₄.5H₂O/Na.Asc/DMF/H₂O/24 h.

The acetonitrile ligand (NCCH₃) was added to the parent complex Re0 (fac-[Re(phen)CO₃(Cl)]) using silver tetrafluoroborate (AgBF₄) in dry acetonitrile. The resulting complex (fac-[Re(phen)CO₃(NCCH₃)]) was then promptly exposed to the 5-(4-ethynylphenyl)-1H-tetrazole ligand and triethylamine, which promoted the exchange of NCCH₃ with the tetrazole ligand. By utilising column chromatography on neutral alumina, the starting materials were eluted with an ethyl acetate solvent system. The product (Re1) was isolated as a bright yellow solid in 78% yield. Reaction chemistry was then employed to link a slight excess of 2-azidoethoxy-α-D-mannose (8), to the alkyne functionalised Re(I) complex (Re1). The copper(I) catalyst was formed in situ using sodium ascorbate to reduce copper(II) sulphate pentahydrate, resulting in the formation of a single 1,4-triazole isomer. Extraction with water and dichloromethane was performed to remove any trace starting materials or reactants, leaving the product as a bright yellow solid in quantitative yield. Analysis by ¹H NMR spectroscopy revealed the appearance of one proton resonance at 8.20 ppm, representing a single 1,4-triazole product. Phenyl and phenanthroline proton resonances exhibited a minor shift upfield by the attachment of the α-D-mannose residue. The formation of a single product was confirmed by ¹³C NMR spectroscopy, which showed 2 carbon resonances from the 1,4-triazole ring at 128.5 and 146.8 ppm.

The synthetic method for Re2 was then applied to attach the azido maltose residue X4 to the Re1 complex. An identical purification procedure was employed to remove any excess starting materials, isolating Re3 (Rhenium maltose complex) as a bright yellow solid. Analysis by ¹H NMR spectroscopy revealed the appearance of proton resonances at 8.44 and 8.86 ppm, representing two regioisomers; the lower energy 1,4-triazole product and the higher energy 1,5-triazole product respectively. The formation of a two isomers was confirmed by ¹³C NMR spectroscopy, which showed 2 carbon resonances at 130.0 and 131.1 ppm (C═CH) from the 1,4- and 1,5-triazole rings respectively. This was a surprising result, as copper(I) catalysed cycloaddition should only produce a single 1,4-triazole product. Each Rhenium complex; Re2 (Rhenium mannose) and Re3 (Rhenium maltose), was then subjected to photophysical examination in aqueous solution in preparation for further investigation in live cells.

2. Photophysical Properties

A dilute solution of the protonated complex in water (5×10⁻⁵ M) with constant ionic strength (I=0.1 M using NaCl) was titrated against NaOH. UV-Visible absorption spectra were recorded over the range of 200-550 nm. Using excitation at 405 nm, fluorescent measurements were recorded over the range of 450-750 nm. Excitation was performed at 405 nm to ensure results were analogous to the 810 nm multiphoton laser excitation used for cellular investigations.

3. Photophysical Properties of Re2 (Rhenium Mannose) in H₂O

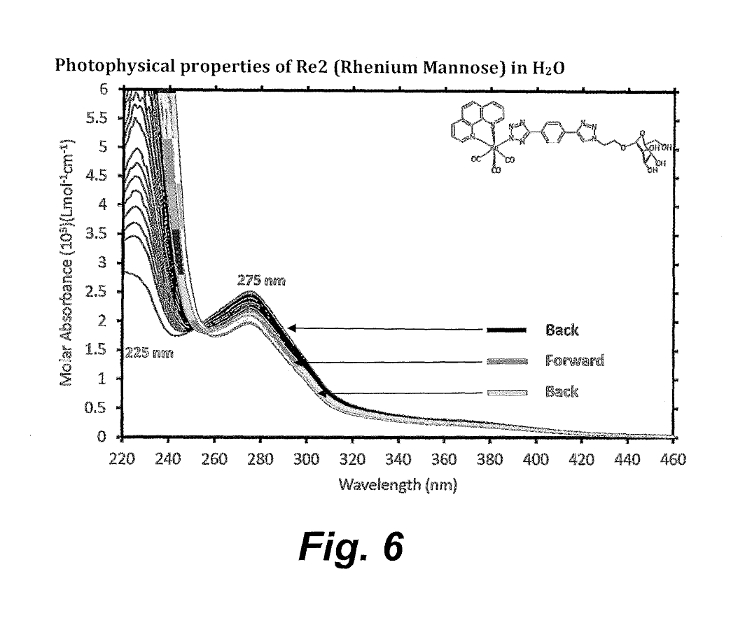

Analysis of the UV-Visible absorbance spectra for Re2 (FIG. 6) showed an intense absorption band at 275 nm and a lower energy band peaking at around 366 nm. The higher energy band at 275 nm was attributed to the spin-allowed, ligand centered π-π* transition, which is localised on the phenyltetrazolato-mannose chelating ligand. The lower energy band in the 360-380 nm region (charge transfer band) was ascribed to a metal-to-ligand charge transfer (MLCT; 5d(Re)→1,10-phenanthroline) mixed with ligand-to-ligand charge transfer character (LLCT; tetrazole→1,10-phenanthroline). As the last two transitions are mixed, they are better described as a metal-ligand-to-ligand charge transfer (MLLCT), a result that is also supported by density functional theory (DFT) calculations.

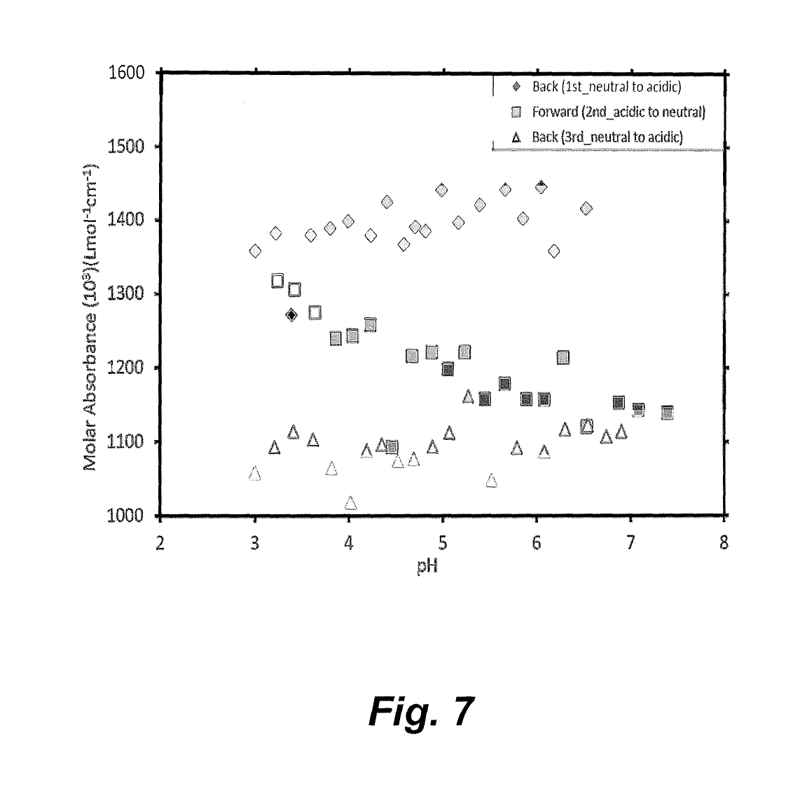

Re2 was found to be only partially soluble in water, and therefore a stock solution of Re2 was made in DMSO then diluted with aqueous solution prior to titration to aid solubility. Starting from a relatively neutral pH of 6.5, acid was added to the aqueous solution, which demonstrated a steady decrease in molar absorptivity as the pH approached 3 (FIG. 7). Upon reversing the pH back to 6.5 the molar absorptivity dropped significantly below the initial reading at 6.5, and continued to decrease when the pH was again adjusted to pH 3. Overall, this suggests that although Re2 may be somewhat pH responsive, the effects were most likely masked by a lack of water solubility, which resulted in the precipitation of Re2 from solution.

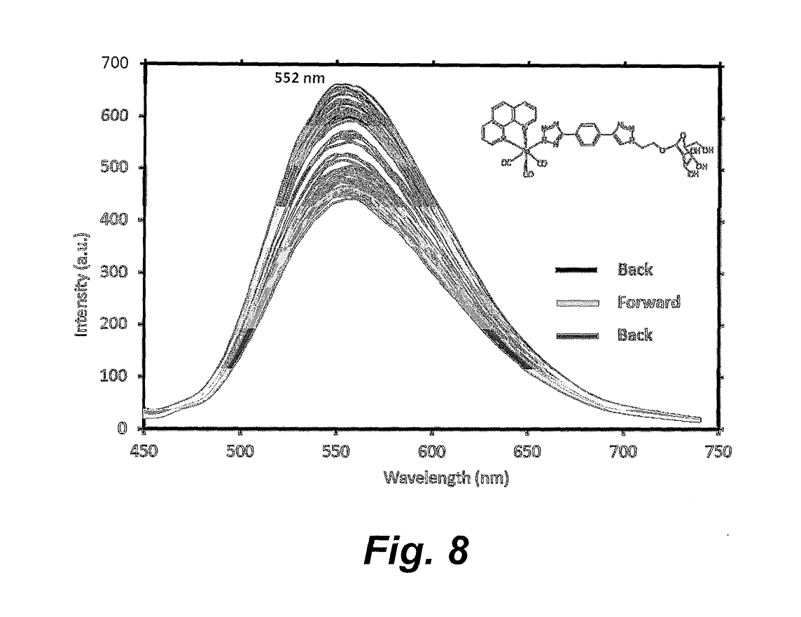

Upon excitation to the lowest singlet 1MLCT band of Re2 (405 nm excitation), a typical broad and structureless emission band, characteristic of the charge transfer nature of the excited state was observed with a single maxima at 552 nm (FIG. 8).

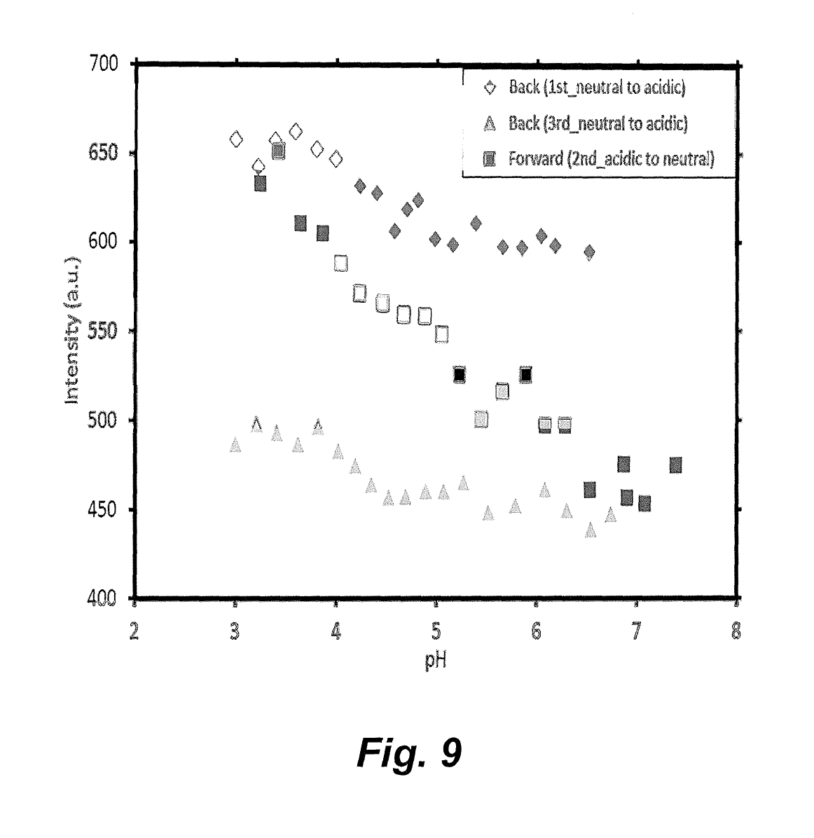

The excited state is characterised by a relatively long lifetime (τ(air)=199 ns, τ(de-oxygenated)=315 ns), suggesting phosphorescent decay from the triplet 3MLCT state. Notably the lifetime decay appears to be mono-exponential for this complex. As expected from the UV-visible spectra, the emission intensity exhibited an overall decrease as the pH was altered between 7 and 3 (FIG. 9). The pH response was not reversible, supporting a lack of water solubility for Re2, which most likely precipitated out of solution reducing the emission intensity over time. As such, photophysical investigations were repeated in a more suitable solvent system; 50:50 MeOH/H₂O.

4. Photophysical Properties of Re2 (Rhenium Mannose) in 50:50 MeOH/H₂O

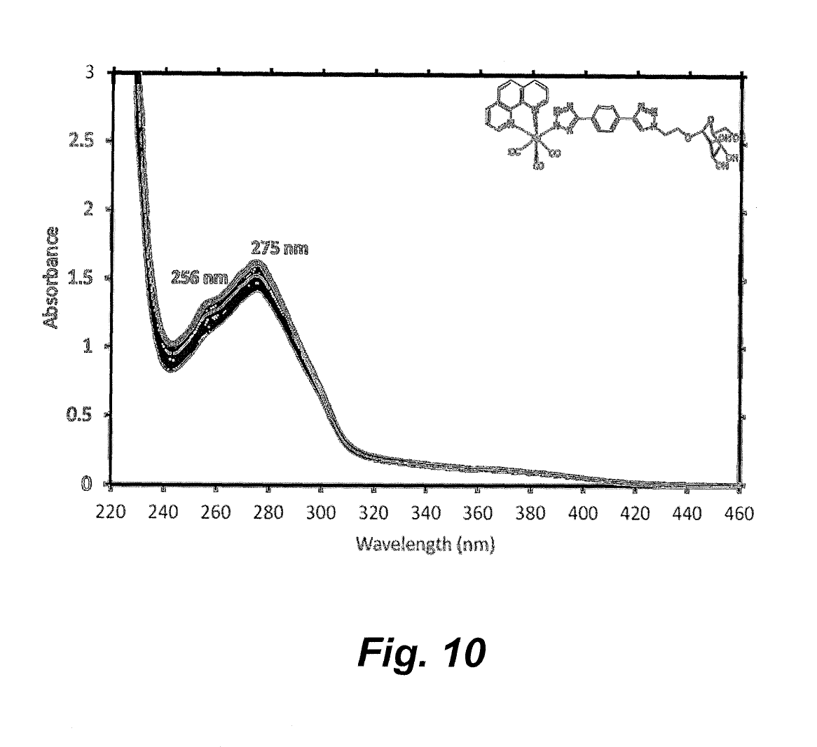

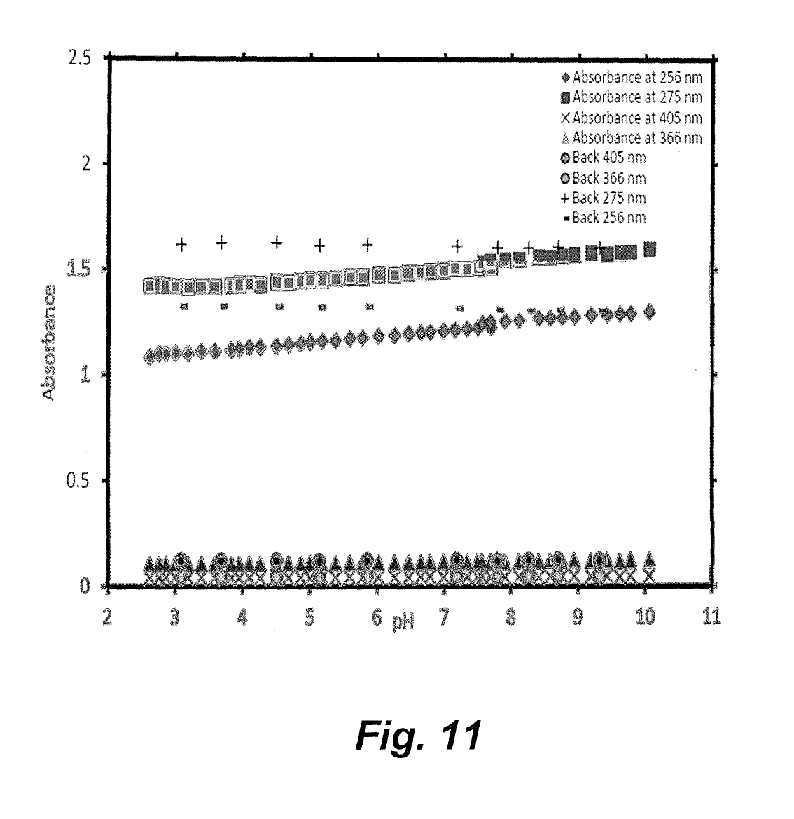

The UV-visible spectra of Re2 in 50:50 MeOH/H₂O displayed a similar pattern to that in 100% H2¬O, with a prominent higher energy band at 275 nm resulting from the spin-allowed, ligand centered π-π* transition localised on the phenyltetrazolato-mannose ligand (FIG. 10). A small shoulder appeared at 256 nm, and is most likely to be the emergence of a triazole π-π* transition, which was previously masked in 100% H₂O. The lower energy charge transfer band in the 360-380 nm region was ascribed to a metal-ligand-to-ligand charge transfer (MLLCT), as described previously. The molar absorptivity of Re2 displayed only a slight increase with decreasing pH, over the pH range of 2-10 (FIG. 11).

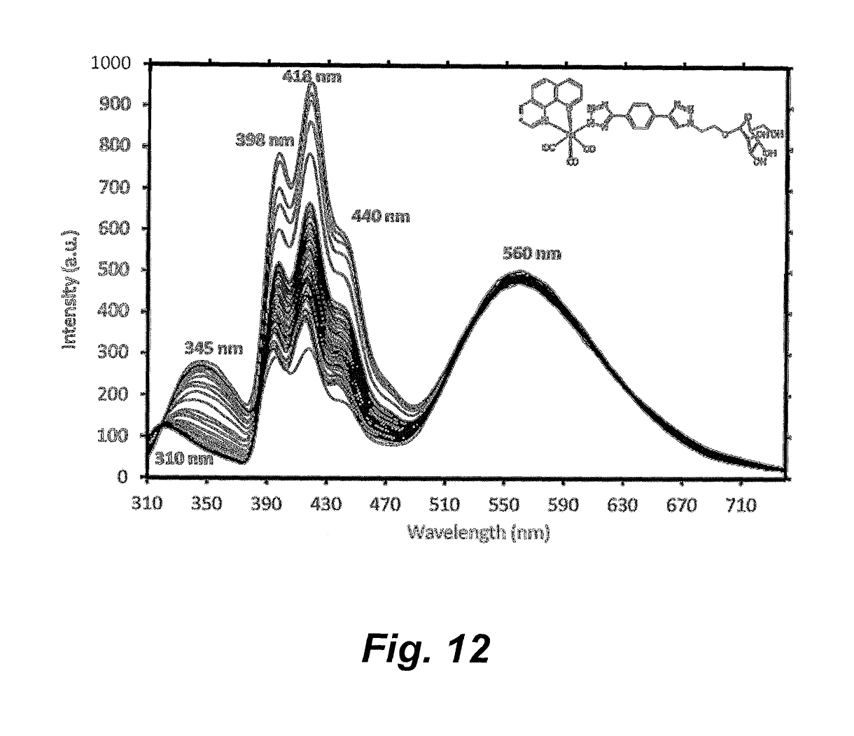

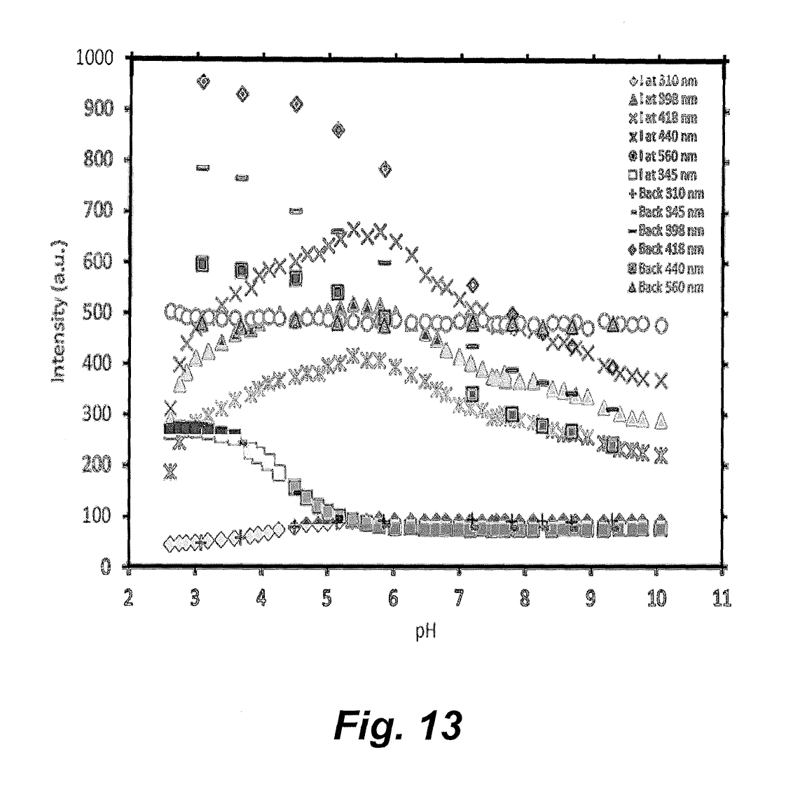

For a full characterisation of the photophysical properties for Re2, a number of different excitation wavelengths were examined. The first investigated was excitation via the π-π* transition of the triazole moiety at 256 nm (FIG. 12). Fluorescent emission from the singlet state of the triazole was observed at 345 nm. Upon decreasing the pH, this emission band underwent a hypsochromic shift to 310 nm, which coincided with a drop in emission intensity (FIG. 13). Further analysis of this transition revealed a pKa value at approximately pH 4.2, corresponding to rapid changes in emission intensity over a small pH range. FIG. 12 also shows 3 emission bands at 398, 418 and 440 nm corresponding to fluorescent emission from the 1,10-phenanthroline π-π* transition. Emission from the 1,10-phenanthroline ligand increased intensity from pH 2 to 6, then decreased from pH 6 to 10. Similar to the emission spectra of Re2 in 100% H₂O, phosphorescent decay from the triplet 3MLCT state was observed at 560 nm. Emission at 560 nm remained stable within the pH range of 3-10.

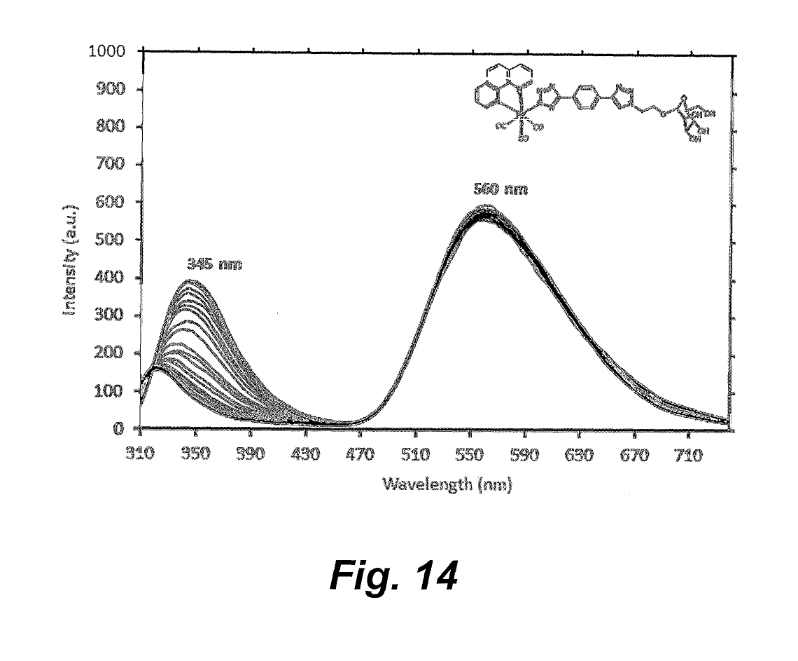

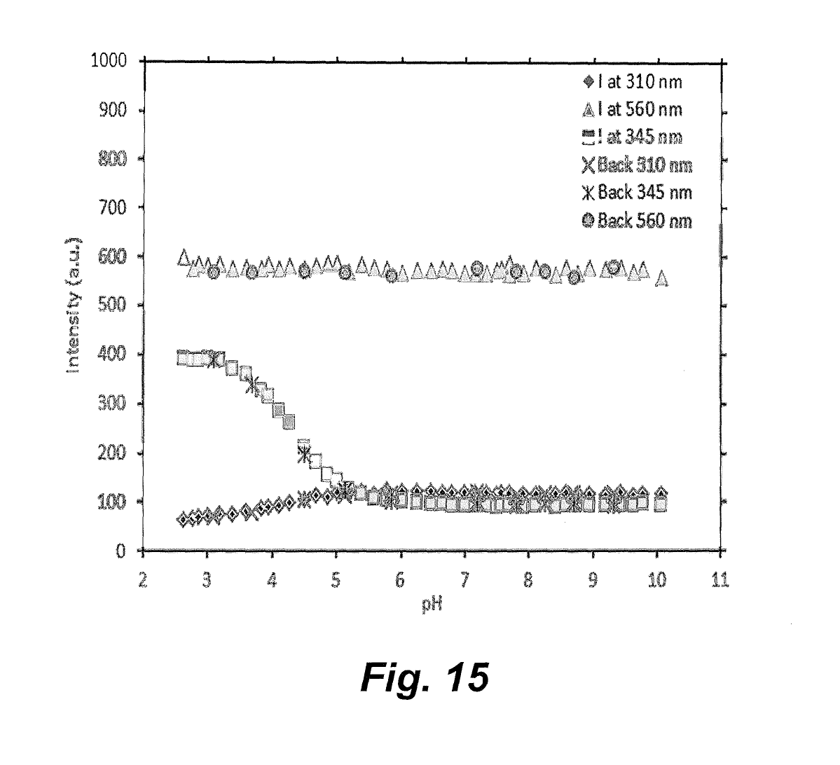

Following the excitation of Re2 at 275 nm, a different emission profile was observed (FIG. 14). Fluorescent emission from the singlet state of the triazole was observed at 345 nm. Upon decreasing the pH, this emission band underwent a hypsochromic shift to 310 nm, which coincided with a drop in emission intensity showing evidence of a pKa around pH 4.2 (FIG. 15). The π-π* emission bands derived from 1,10-phenanthroline ligand were not observed using 275 nm excitation. Phosphorescent emission from the triplet ³MLCT state was observed at 560 nm, and remained stable within the pH range of 3-10. Importantly the pH titration using 275 nm excitation was fully reversible in the forward and backward direction, confirming the complex requires some organic solvent for solubility at the concentration ranges required for these photophysical measurements.

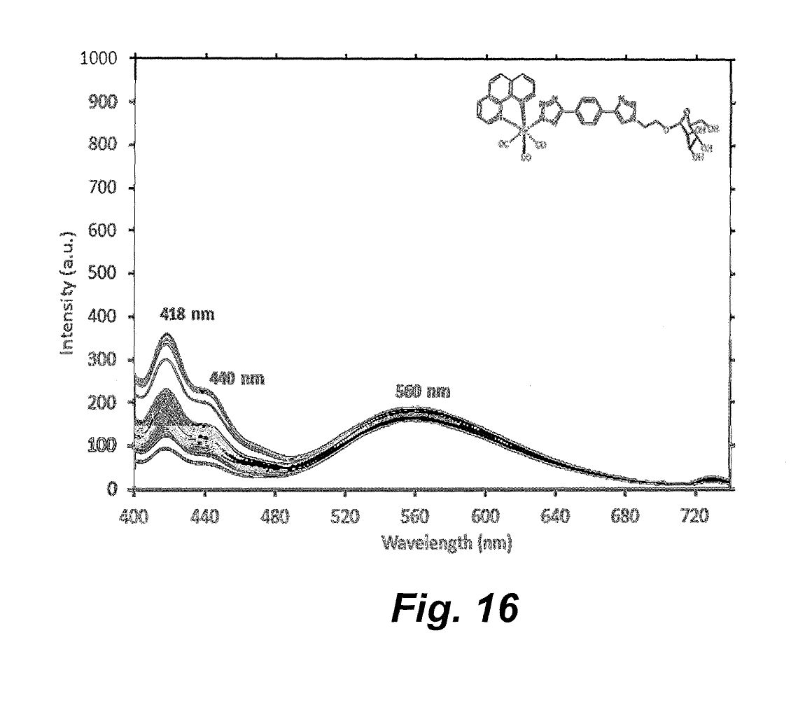

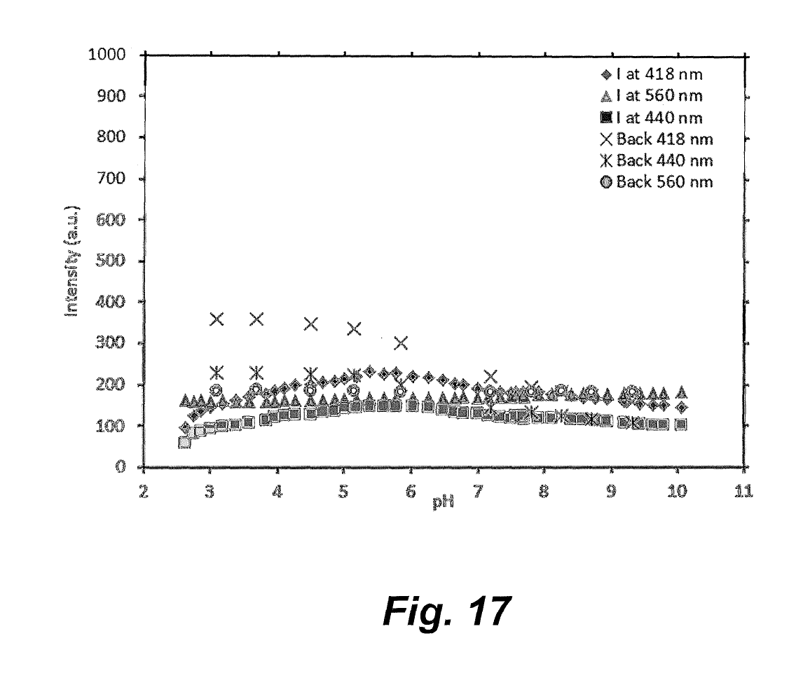

Moving to a lower energy excitation wavelength of 366 nm, emission from the π-π* orbital of the 1,10-phenanthroline ligand is observed at 418 and 440 nm (due to detection constraints), whilst phosphorescence from the triplet 3MLCT state was observed at 560 nm (FIG. 16). Emission at 560 nm remained stable within the pH range of 3-10, whereas emission from the 1,10-phenanthroline ligand increased intensity from pH 2 to 6, then decreased from pH 6 to 10 (FIG. 17).

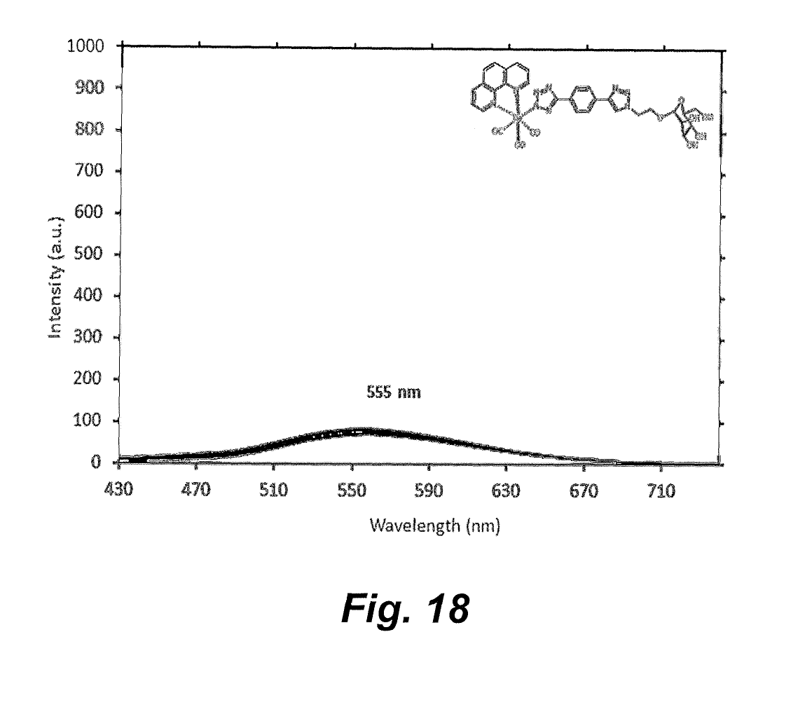

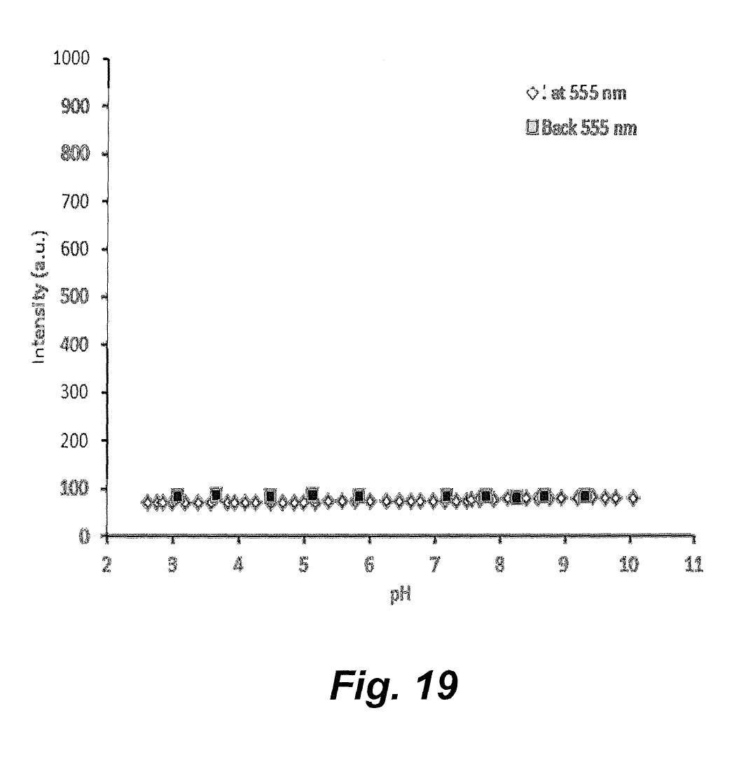

Finally, an excitation wavelength of 405 nm was employed for a comparison between the cellular emission of Re2 derived from 810 nm two-photon excitation. The emission spectra of Re2 were characteristic of phosphorescence from the triplet 3MLCT state at 555 nm (FIG. 18). Emission at 555 nm remained stable within the pH range of 3-10 (FIG. 19). At this excitation wavelength Re2 can be considered to be pH insensitive.

5. Photophysical Properties of Re3 (Rhenium Mannose) in 50:50 MeOH/H₂O

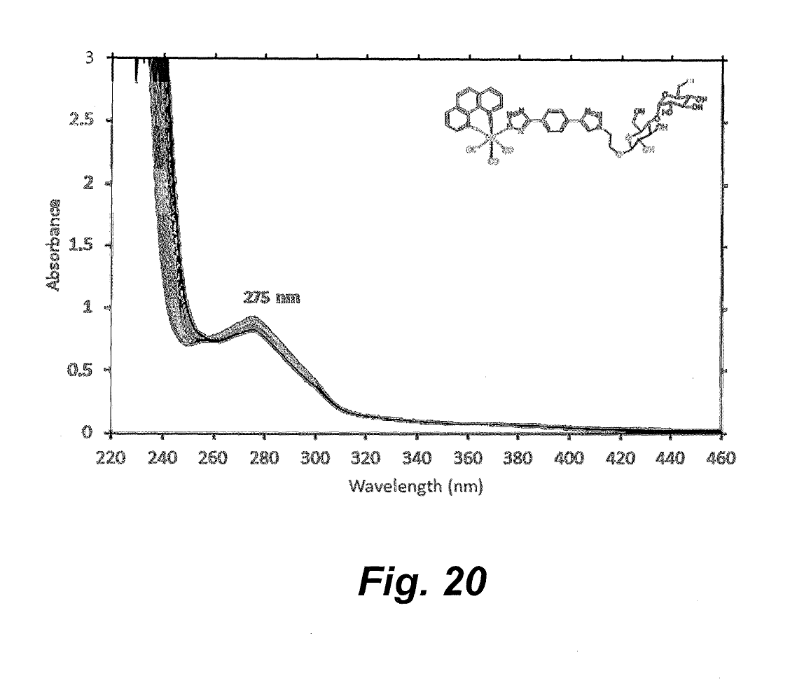

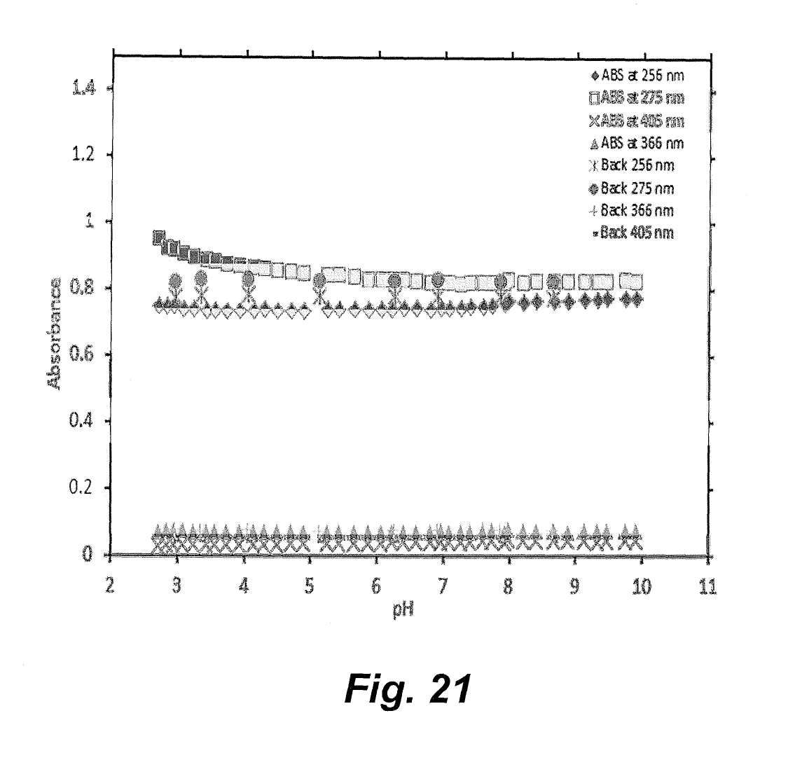

The UV-visible spectra of Re3 in 50:50 MeOH/H₂O displayed a similar pattern to that of Re2 in 50:50 MeOH/H₂O with a prominent higher energy band at 275 nm resulting from the spin-allowed, ligand centered π-π* transition localised on the phenyltetrazolato-maltose ligand (FIG. 20). A shoulder was not observed at 256 nm, as it was for Re2. The lower energy charge transfer band in the 360-380 nm region was ascribed to a metal-ligand-to-ligand charge transfer (MLLCT), as described previously. The molar absorptivity of Re3 displayed only a slight decrease with decreasing pH, over the pH range of 2-10 (FIG. 21).

For a full characterisation of the photophysical properties for Re3, a number of different excitation wavelengths were examined. The first investigated was excitation via the π-π* transition of the triazole moiety at 256 nm (FIG. 20). Fluorescent emission from the singlet state of the triazole was observed at 345 nm. Upon decreasing the pH, this emission band underwent a hypsochromic shift to 320 nm, which coincided with a drop in emission intensity (FIG. 21). Further analysis of this transition revealed a pKa value at approximately pH 4.2, corresponding to rapid changes in emission intensity over a small pH range. FIG. 20 also shows 3 emission bands at 398, 418 and 440 nm corresponding to fluorescent emission from the 1,10-phenanthroline π-π* transition. Emission from the 1,10-phenanthroline ligand increased intensity from pH 2 to 6, then decreased from pH 6 to 10. Similar to the emission spectra of Re2 in 100% H2O, phosphorescent decay from the triplet ³MLCT state was observed at 560 nm. Emission at 560 nm remained stable within the pH range of 3-10.

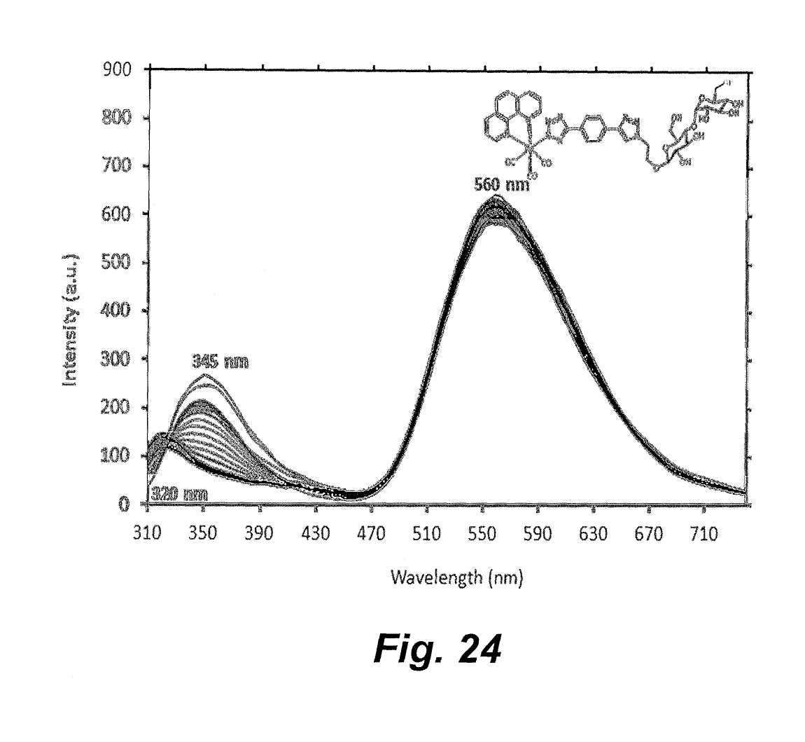

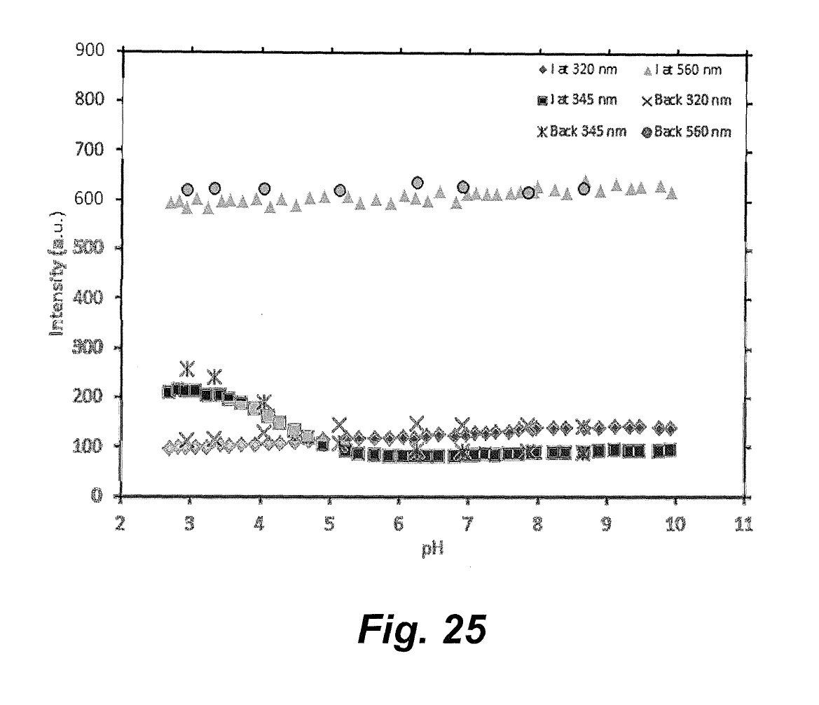

Following the excitation of Re3 at 275 nm, an emission profile comparable to Re2 at the same emission wavelength was observed (FIG. 24). Fluorescent emission from the singlet state of the triazole was observed at 345 nm. Upon decreasing the pH, this emission band underwent a hypsochromic shift to 320 nm, which coincided with a drop in emission intensity. As with 256 nm excitation, there was evidence of a pKa around pH 4.2 (FIG. 25). The π-π* emission bands derived from 1,10-phenanthroline ligand were not observed using 275 nm excitation. Phosphorescent emission from the triplet 3MLCT state was observed at 560 nm and remained stable within the pH range of 3-10. Importantly the pH titration using 275 nm excitation was fully reversible in the forward and backward direction, confirming the complex requires some organic solvent for solubility at the concentration ranges required for these photophysical measurements.

A lower energy excitation wavelength of 366 nm, showed emission from the π-π* orbital of the 1,10-phenanthroline ligand at 418 and 440 nm (due to detection constraints), whilst phosphorescence from the triplet ³MLCT state was observed at 560 nm (FIG. 24). Emission at 560 nm remained stable within the pH range of 3-10, whereas emission from the 1,10-phenanthroline ligand increased intensity from pH 2 to 6, then decreased from pH 6 to 10 (FIG. 25).

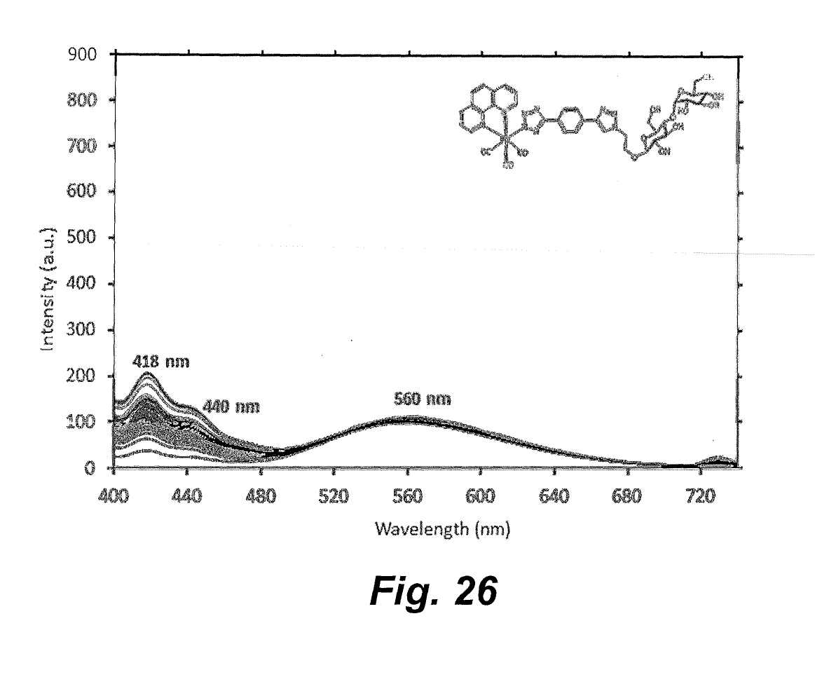

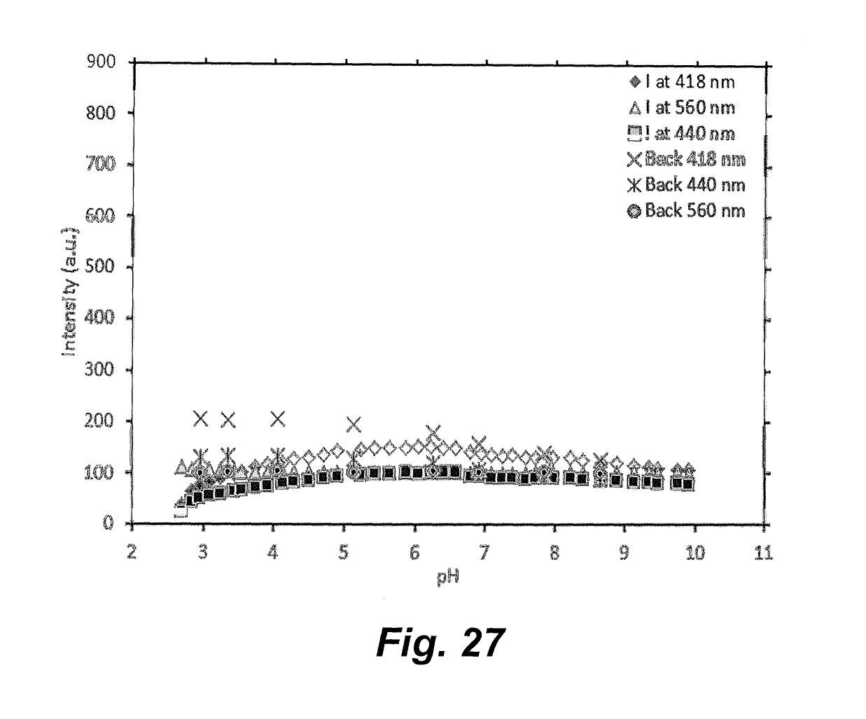

Finally, an excitation wavelength of 405 nm was employed, displaying emission spectra for Re3 which were characteristic of phosphorescence from the triplet 3MLCT state at 555 nm (FIG. 26). Emission at 555 nm remained stable within the pH range of 3-10 (FIG. 27).

Quantum yields for the complexes Re2 and Re3 were calculated and are shown below in Table 1. Lifetimes for Re2 and Re3 were relatively long, indicating the phosphorescent nature of the rhenium emission. Quantum yields for Re2 and Re3 were comparable to those found in the literature for rhenium-tetrazolato complexes.

TABLE 1
Lifetimes and quantum yields for complexes Re2 and Re3

| | | | Quantum | Quantum |
|---|---|---|---|---|
| | Lifetime: Air | Lifetime: de- | Yield Air | Yield: de- |
| Complex | equilibrated | oxygenated: | equilibrated | oxygenated |
| Re2 | 199 ns | 315 ns | 1.64% | 8.26% |

Both Re2 and Re3 displayed evidence of a pKa at approximately 4.2. Re2 and Re3 showed very similar absorption and emission profiles, and their emission intensities were comparable. Coordinating mannose and maltose to a complex of the type fac-[Re(I)(phen)(CO)3(Talk)] (Re1) showed no adverse effects on rhenium phosphorescence. Using 405 nm, emission from Re2 and Re3 was shown to be sufficient for cellular imaging investigations.

5. Cellular Localisation of Re2 (RB124, Rhenium Mannose) in Live Drosophila Fat Body Cells

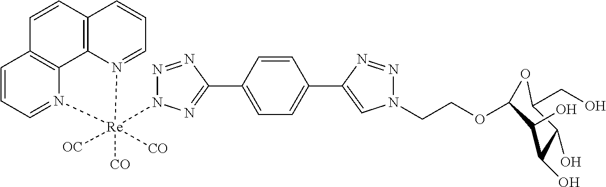

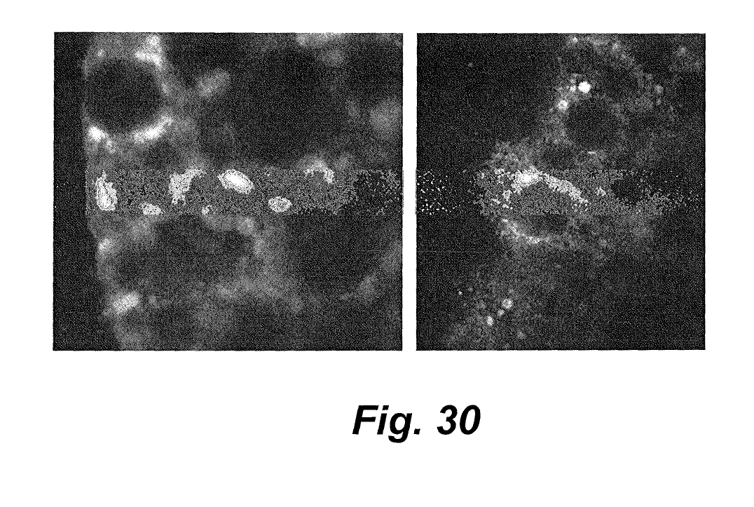

The Rhenium complex Re2 was investigated in Drosophila fat body cells to define the uptake and distribution in live cells. Two-photon excitation at 810 nm was employed to reduce cellular damage and autofluorescence, whilst producing in vivo photophysical properties analogous to those measured from dilute solutions. With an effective excitation wavelength of 405 nm, the emission maxima of the signal was expected to be 560 nm, therefore cellular images were collected over the range of 425-750 nm. Following a 15 minute incubation, Re2 was internalised into Drosophila fat body cells and localised to vesicular compartments in fat body cells, but excluded from lipid droplets (FIG. 30).

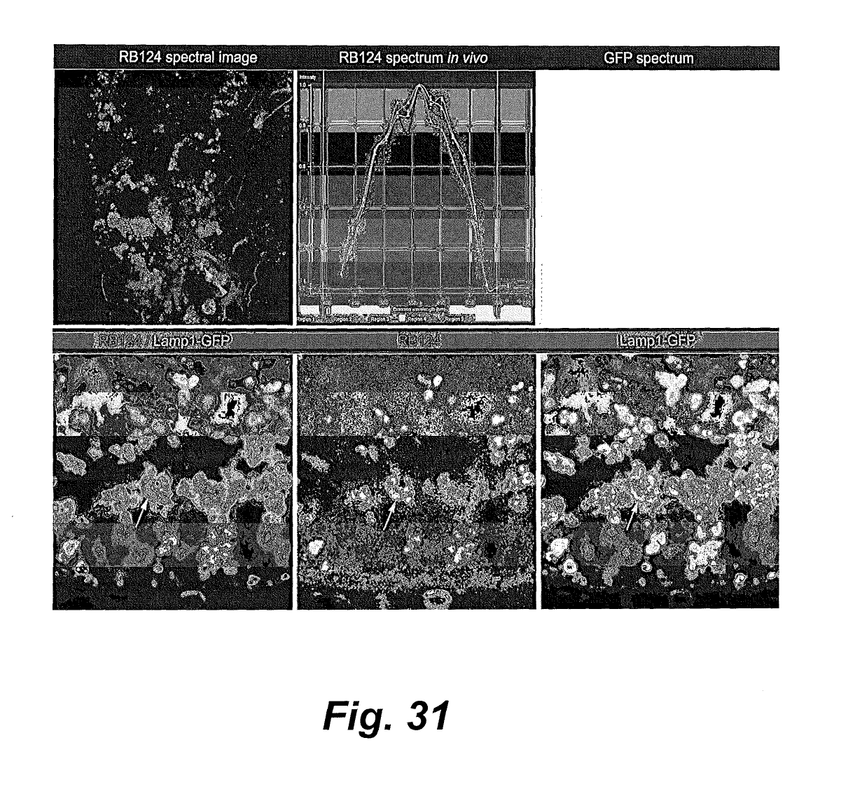

The subcellular distribution of Re2 was further defined in Drosophila fat body cells, by incorporating Re2 into fat body cells expressing the lysosomal marker, Lysosome Associated Membrane Protein 1-Green Fluorescent Protein (LAMP1-GFP). Following a 15 minute incubation, fluorescence from Re2 was observed as punctate vesicular staining, which was colocalised with compartments containing LAMP1-GFP (FIG. 31).

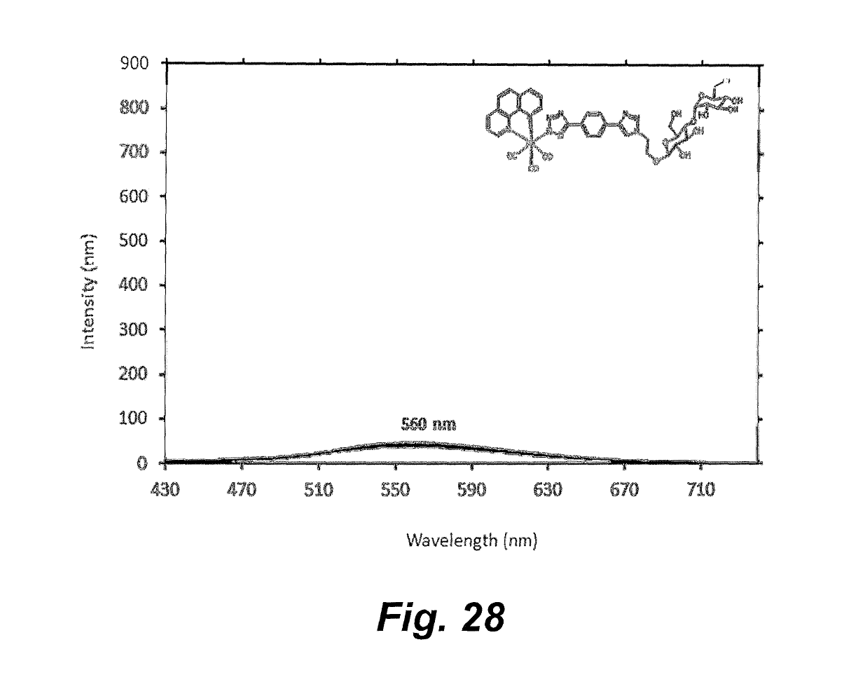

Although there were some LAMP1-GFP-positive vesicles that were not Re2 positive, there were no vesicles containing Re2 that were not LAMP1-GFP positive. This confirmed that after cellular internalisation, Re2 was localised within lysosomal compartments. The spectral profile of the Re2 emission in vivo showed a single maximum around 560 nm (FIG. 31: A′), which was consistent with the emission spectra of Re2 recorded in 50:50 MeOH/water (FIG. 28). This indicated that the complex remained intact within the cellular environment.

Uptake via receptor mediated endocytosis would place Re2 in endosome-like compartments at approximately 15 minutes, followed by lysosomal compartments at 30-45 minutes. As Re2 was located within lysosomes after 15 minutes, this suggested that the method of internalisation was passive diffusion across the membrane and therefore not receptor mediated endocytosis. Although the mannose targeting motif did not appear to control the uptake mechanism, the overall structure of the probe still resulted in a specific distribution. A relatively non-polar Rhenium(I) tetrazolato complex might be expected to pass across the cell membrane via passive diffusion and partially localise within lipid droplets. The biological probe Re2 was excluded from lipid droplets suggesting that its hydrophilicity was sufficient enough to prevent this. Re2 is also a relatively small biological probe, which may explain why it is transported rapidly across the cell membrane.

6. Cellular Localisation of Re3 (RB155, Rhenium Maltose) in Live Drosophila Fat Body Cells

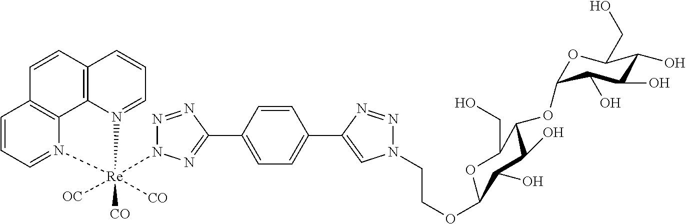

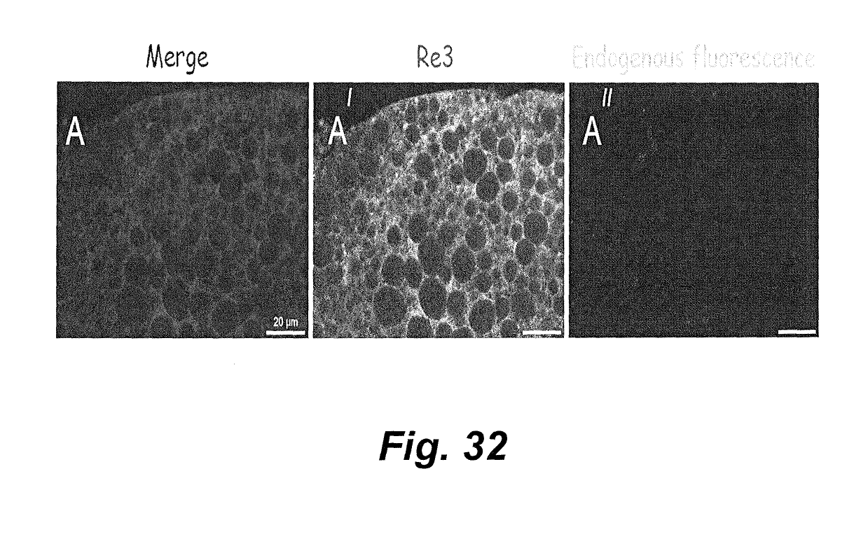

The probe Re3 (Rhenium maltose complex) was internalised by Drosophila fat body cells following a 15 minute incubation, then distributed into the cytosol (FIG. 32: A-A″). As with the biological probe Re2 (Rhenium mannose), Re3 was excluded from lipid droplets. There was little or no endogenous fluorescence at 810 nm in fat body cells, whereas the 425-750 nm fluorescence detected from Re3 was distinct. Re3 fluorescence was mainly detected as diffuse cytosolic staining, though there were areas of punctate staining in which the probe appeared to be more concentrated.

7. Cellular Localisation of Re3 in THP-1 Macrophages

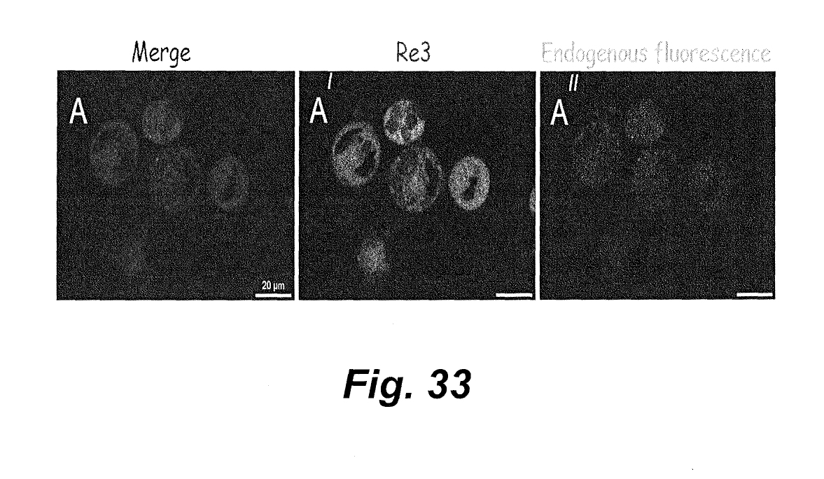

The probe Re3 (Rhenium maltose complex) was internalised by THP-1 macrophages following a 15 minute incubation, then distributed throughout the cytosol (FIG. 33: A-A″). Punctate fluorescence from Re3 was observed over the range of 425-750 nm, whilst minimal or no endogenous fluorescence was detected.

8. Cellular Localisation of Re3 in Prostate Cells

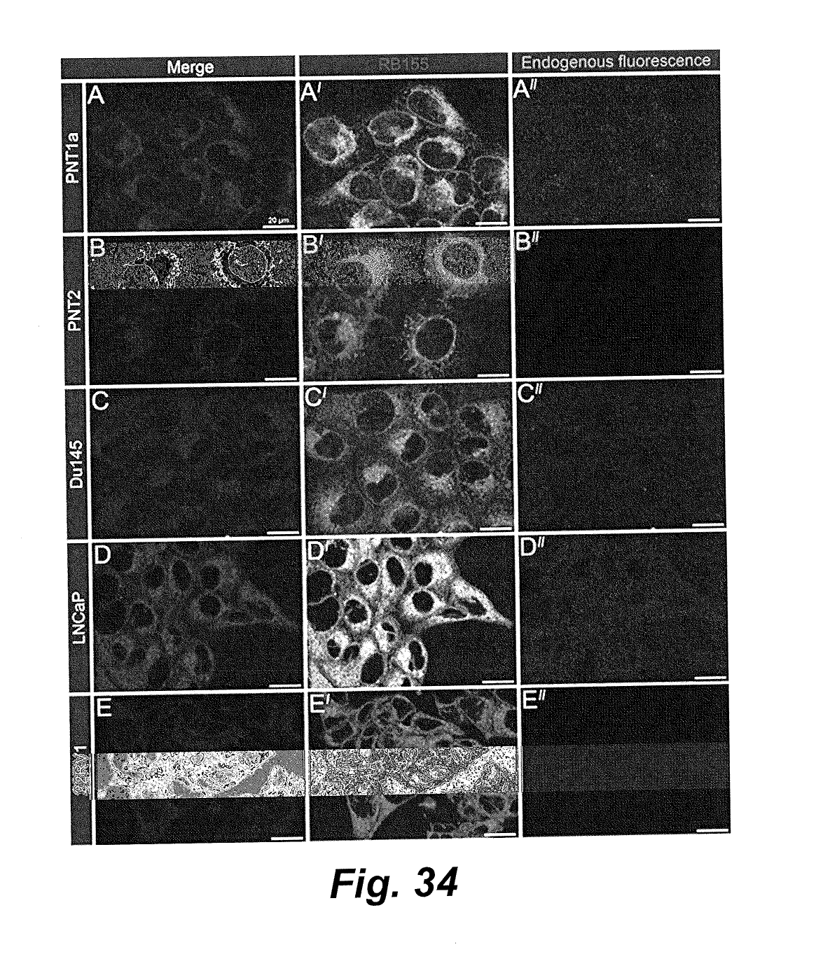

The probe Re3 was also internalised by control prostate and prostate cancer cells (FIG. 34). Interestingly, the amount of uptake was greater in prostate cancer cell lines compared to the control non-malignant cell lines. In addition, the distribution of Re3 in control cells was mainly perinuclear in a diffuse pattern radiating from the nucleus. In contrast, Re3 was detected throughout the cytoplasm of the prostate cancer cells and appeared to also be localised in punctate structures within the cytosol.

The Re3 probe was internalised and distributed into the cytosol of Drosophila fat body cells by 15 minutes, suggesting that it is rapidly transported into these cells. The targeting motif on the Re3 probe was maltose; a sugar that is normally transported through the plasma membrane of cells by GLUT transporters. For example, the GLUT1 and GLUT12 transporters are able to transport glucose and maltose sugars, enabling the intracellular incorporation of these substrates into energy utilisation pathways. The rough endoplasmic reticulum is a site for glucose storage in cells, and the staining pattern in the cytosol of the control prostate cells was consistent with this distribution. The GLUT1 transporter shows increased expression in prostate cancer cells, when compared to control prostate cell lines and this could account for the increased uptake of the Re3 probe in these cancer cells. The altered intracellular distribution of the Re3 probe in prostate cancer cells may be an indication of the altered energy utilisation and alternative pathways indicative of rapidly dividing cancer cells. Increased uptake in androgen sensitive prostate cancer cell lines suggests that these cell lines are more dependent on glucose metabolism. The development of androgen insensitivity is a major clinical problem during hormone ablation therapy. The probe Re3 may be a useful indicator for cells that are changing androgen sensitivity and altering their metabolism.

9. Conclusion

A versatile synthesis for the production of Re(I) tetrazolato complexes of the type fac-[Re(phen)CO3(Talk)] (where phen=1,10-phenanthroline and Talk=5-(4-ethynylphenyl) tetrazole) was developed. By utilising this terminal alkyne, Re(I) complexes were successfully linked to mannose and maltose targeting moieties. The resulting complexes Re2 (mannose targeted) and Re3 (maltose targeted) were photophysically investigated and found to be suitable for cellular imaging. Re(I) tetrazolato complexes functionalised with a mannose residue localised within lysosomes of Drosophila fat body cells, whereas those functionalised with maltose were distributed throughout the endoplasmic reticulum, acidic vesicles (e.g. lysosomes) and cytoplasm. Re2 and Re3 displayed similar cellular distribution throughout the endoplasmic reticulum and cytoplasm in THP-1 macrophages and non-malignant prostate cell lines PNT1a and PNT2. Interestingly, Re2 and Re3 displayed altered cellular distribution in malignant prostate cancer cell lines; DU145, 22RV1 and LNCaP, where localisation was observed in the extracellular compartments for Re2 and perinuclear region (most likely in the ER) for Re3. This differential localisation demonstrates that in these complexes, organelle specificity can be achieved and manipulated by functional group transformations.

### Example 2—Cellular Localisation of Re2 in Prostate Cells

The probe Re2 identified cancerous prostate cells on the basis of altered labelling of the intracellular and the extracellular structures of the cells studied.

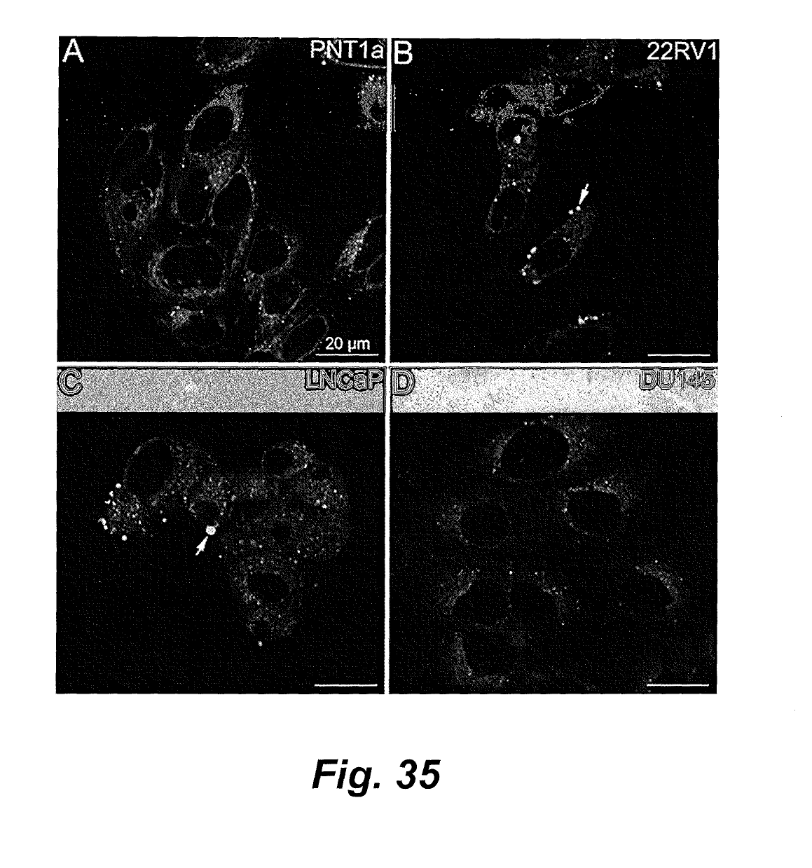

Cancer cells show enhanced sugar uptake and glycolytic rates compared to non-malignant cells. Therefore, the uptake and distribution of Re2 by live human prostate cancer cells (22RV1, LNCaP and DU145) and non-malignant control PNT1a was investigated, and the data is presented in FIG. 35. Treatment of non-malignant PNT1a and three prostate cancer cell lines with Re2 (20 μM) in a glucose-free and fetal calf serum-free medium at 37° C. and 5% CO₂ for 30 minutes resulted in accumulation of the probe in the perinuclear region, intracellular and extracellular compartments.

The probe Re2 was internalised by prostate non-malignant and cancer cells following a 30 minute incubation, and it was distributed throughout the cytosol, with greater accumulation in the extracellular compartments. perinuclear region (FIG. 35).

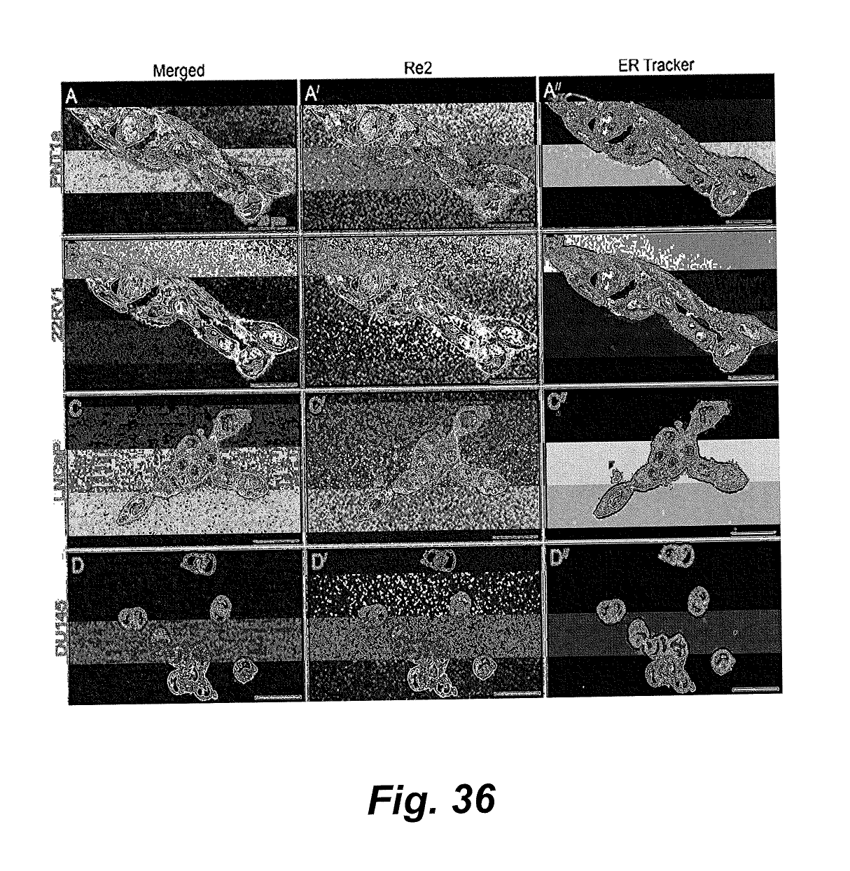

The intracellular distribution of the probe Re2 was further defined in prostate cancer cells by co-staining cells with commercial dye, ER-Tracker™ Red (BODIPY® TR Glibenclamide), which was used for the detection of the endoplasmic reticulum in live cells. Our results showed (FIG. 36) that the probe Re2 co-localised with ER-Tracker™ Red, and this included their co-localisation at the nuclear membrane and on the reticular network extending into the cytoplasm from the nucleus.

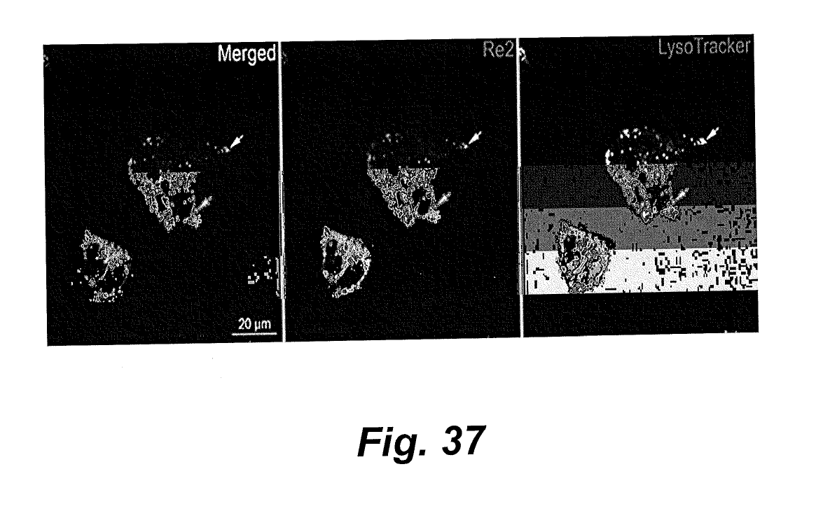

Also, there was some albeit limited amount of Re2 detected in association with lysosomes/acidic compartments. This was confirmed by co-staining cells with the commercial dye LysoTracker® Red DND-99 (1:1000 dilution, 2 minutes incubation at 37° C. and 5% CO₂), which is known to label lysosomes/acidic compartments in live cells (FIG. 37).

The probe Re2 showed different staining patterns between non-malignant control PNT1a and prostate cancer (22RV1, LNCaP and DU145) cells. In cancer 22RV1, LNCaP and DU145 cells, Re2 accumulated in large cell surface associated structures and had increased perinuclear staining.

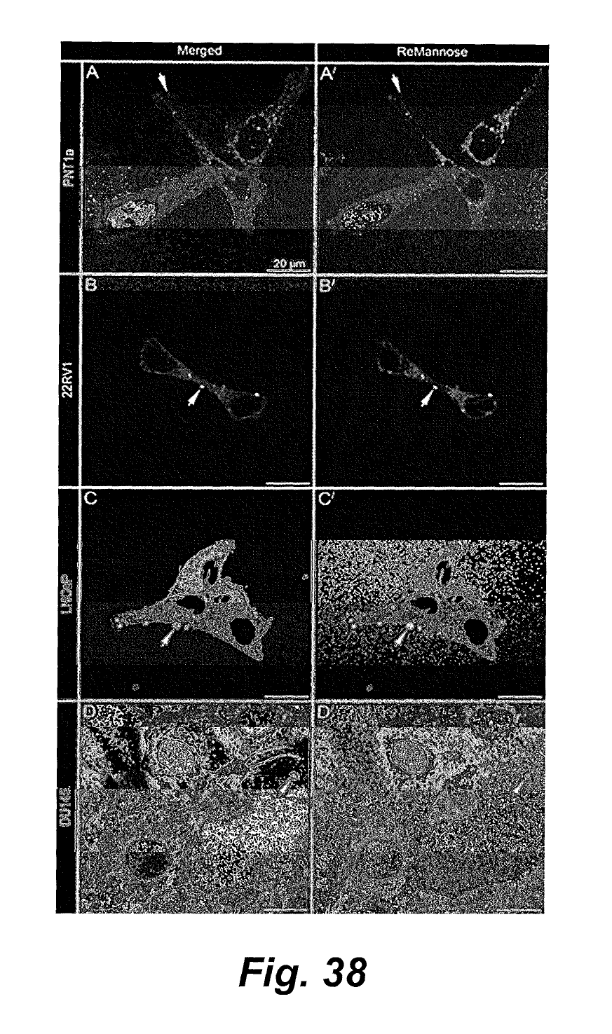

FIG. 38 shows that Re2 accumulates in cell surface associated compartments of prostate cells. (A-D) Micrographs of cross-section through prostate non-malignant control PNT1a (A, A/) and cancer (B, B/—22RV1; C, C/—LNCaP; and D, D/—DU145) cells showing Re2 (green in A-D and greyscale in A/-D/) in the extracellular compartments. The plasma membrane was outlined by CellMask™ deep Red (red in A-D). Arrows depict extracellular compartments. Scale bar, 20 μm.

### Example 4—Cellular Localisation of Re2 in Human THP-1 Macrophages

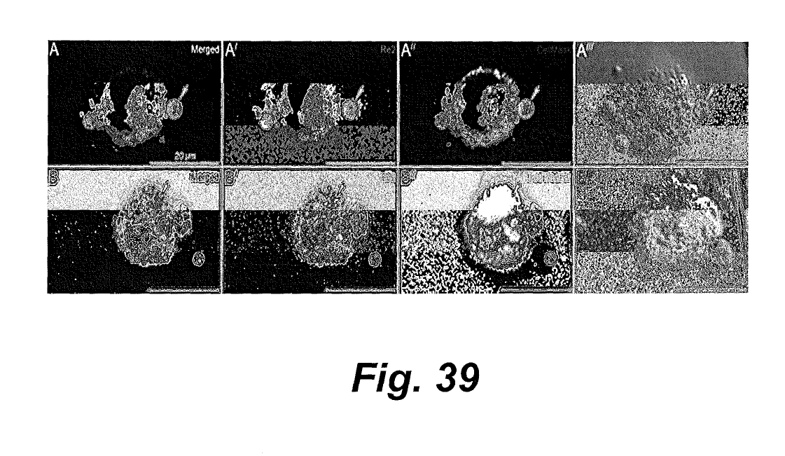

FIG. 39 shows Re2 accumulates in secretory vesicles and lysosomes in live THP-1 macrophages. (A-A^{//}). Micrographs of cross-section through macrophages showing accumulation of Re2 (green in A, greyscale in A^{/)} in secretory vesicles, which membrane was outlined by CellMask™ Deep Red (red in A, greyscale in A^{//}). Arrow in A-A^{///} depicts these Re2-positive secretory vesicles. (B-B^{//}) Micrographs of cross-section through THP-1 macrophages showing co-localisation of Re2 (green in A, greyscale in A^{/)} and LysoTracker® Red DND-99 (red in B, greyscale in B^{//}red). Arrow in B-B^{///} shows lysosomes in which Re2 has been observed. Brightfield images of THP-1 macrophages are in A^{///} and B^{///}. Scale bar, 20 μm.

### Example 5—Cellular Localisation of Re3 in H9c2 Rat Cardiomyoblasts

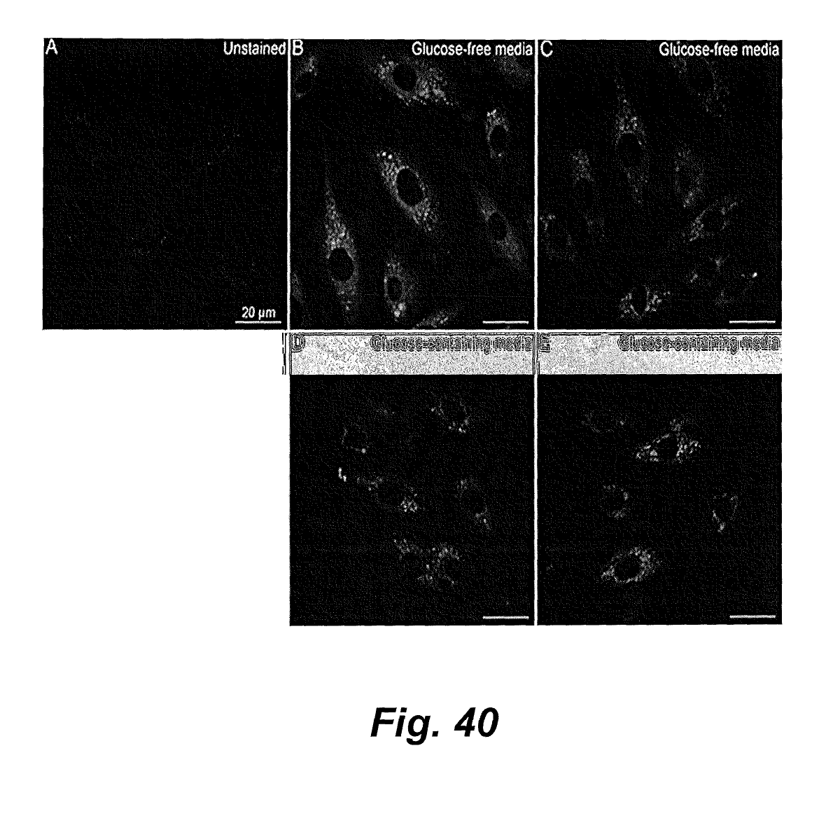

FIG. 40 shows the subcellular localisation of Re3. (A) Confocal micrograph showing unstained H9c2 cells. (B-E) Micrographs of cross-section through H9c2 rat cardiomyoblasts showing intracellular distribution of Re3. (B, C) The cells were stained with Re3 in D-glucose-free and FCS-free media for 30 minutes. (D, E) The cells were stained with the probe in media containing D-glucose. The H9c2 cells were imaged after they were washed from the probe (C, E) and prior to it (B, D). Scale bar, 20 μm.

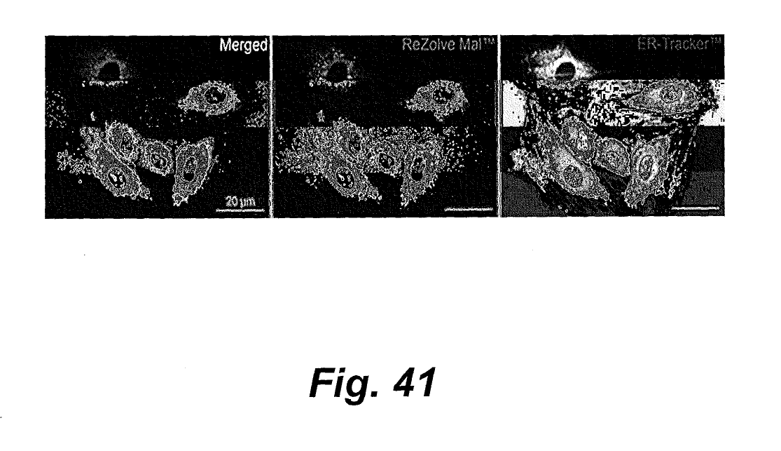

FIG. 41 shows Re3 localises to the endoplasmic reticulum in H9c2 cells. Confocal micrographs showing co-localisation of the probe Re3 and ER-Tracker™ in the H9C2 rat cardiomyoblasts. Scale bar, 20 μm.

### Example 6—Cellular Localisation of Re3 in Healthy and Infarcted Myocardium

The left ventricles from healthy and infarcted lambs were stained for one hour with Re3, and then imaged in live mode under confocal microscopy, which was supplemented with a two-photon Mai-Tai® laser.

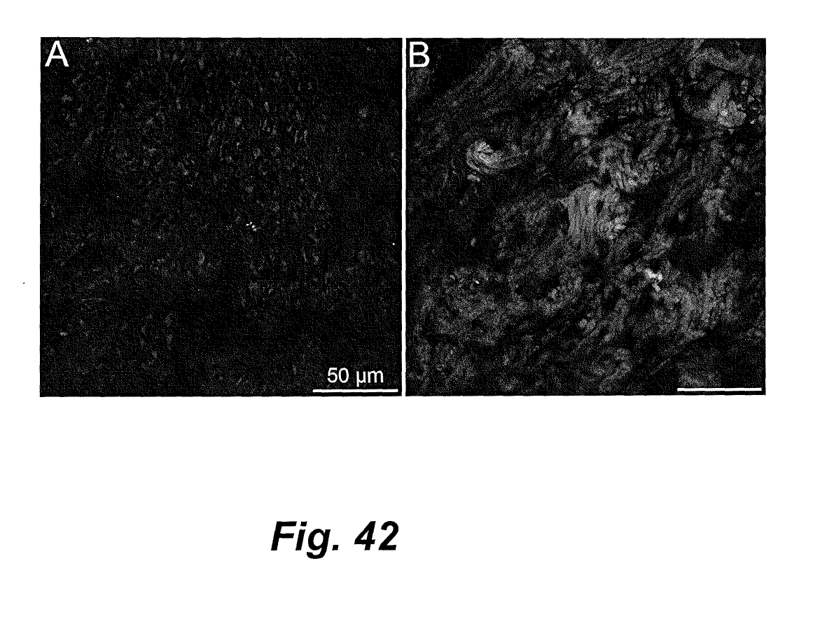

FIG. 42 shows Re3 imaging of infarction in lamb. Micrographs of cross-section through the myocardium showing distribution of Re3. Representative images were from healthy (A) and infarcted myocardium (B) excised from lambs. Scale bar, 50 μm.

### Example 7—Cellular Localisation of Re2 in Healthy Muscle Tissues

The muscles (quadriceps) from healthy lambs were stained with 20 uM Re2 for 30 minutes at room temperature, rinsed with PBS and then imaged by using confocal microscope (excitation, 403 nm/emission, 505-625 nm).

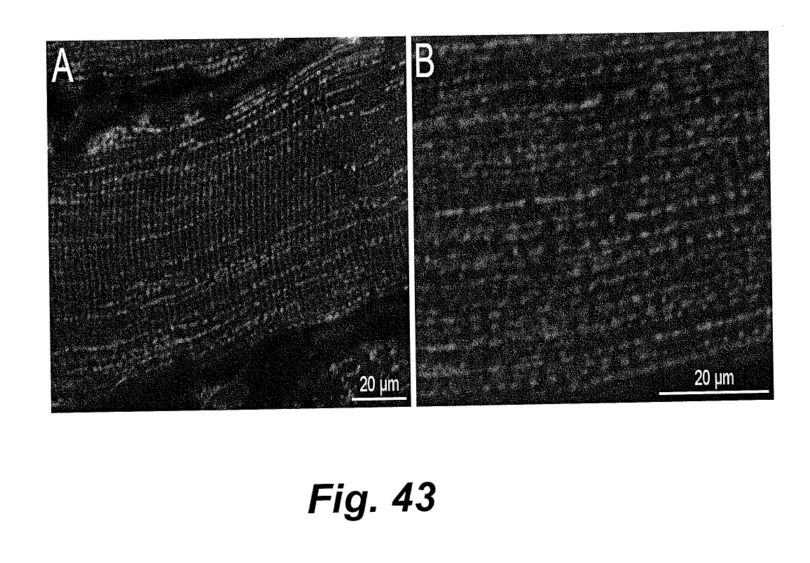

The data is shown in FIG. 43, being confocal micrographs showing intracellular localisation of Re2 in lamb quadriceps. Representative images were from healthy lambs. Scale bar, 20 μm.

### Example 8—Synthesis of Re4 and Re5

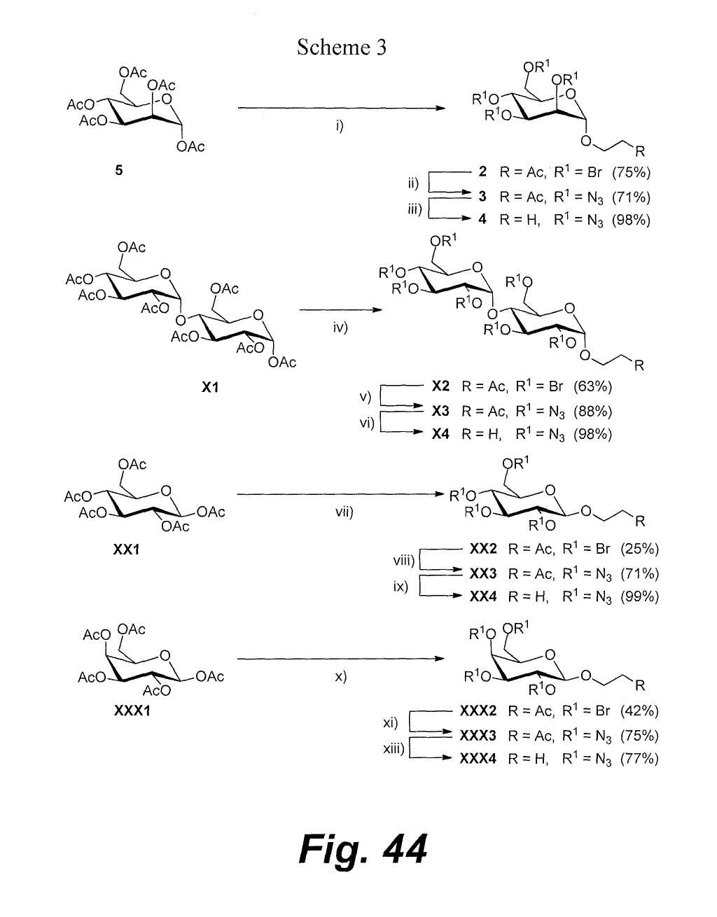

Reagents and conditions for Scheme 3 (FIG. 44): i) 2-Bromoethanol/BF₃.Et₂O/CH₂Cl₂/24 h/0→25° C.; ii) NaN₃/DMF/100° C./1.5 h; iii) NaOMe/MeOH/24 h; iv) 2-Bromoethanol/BF₃.Et₂O/CH₂Cl₂/24 h/0→25° C.; v) NaN₃/DMF/100° C./1.5 h; vi) NaOMe/MeOH/24 h; vii) 2-Bromoethanol/BF₃.Et₂O/ZnCl₂CH₂Cl₂/24 h/0→25° C.; viii) NaN₃/DMF/100° C./1.5 h; ix) NaOMe/MeOH/24 h; x) 2-Bromoethanol/BF₃.Et₂O/ZnCl₂CH₂Cl₂/24 h/0→25° C.; xi) NaN₃/DMF/100° C./1.5 h; xii) NaOMe/MeOH/24 h.

2-azidoethoxy-α-D-mannose (8) was synthesised as follows: α-D-mannose pentaacetate (5) was alkylated using 2-bromoethanol (6), converted to an azide (7) with NaN₃ and then deacetylated using NaOMe. Compound 5 was alkylated with 2-bromoethanol in the presence of boron trifluorodiethyletherate (BF₃.Et₂O) at 0° C. to produce a thick syrup. The syrup was crystallised from Et₂O/CH₂Cl₂ and the desired bromide (6) was isolated as white crystals in 75% yield. Substitution of the C1-acetyl group with 2-ethoxy bromide was confirmed using ¹H NMR spectroscopy. An upfield shift of the H1 resonance from 6.00 to 4.88 ppm was observed and the appearance of two methylene resonances (an apparent doublet of triplets at 3.87 ppm (OCH₂) and an apparent triplet at 3.52 ppm (CH₂Br)) indicated reaction success. High resolution mass spectrometry (HRMS) confirmed the successful formation of compound 6 with a detected m/z at 477.0364 (M+Na)⁺, calculated for C₁₆H₂₃⁷⁹BrO₁₀ (M+Na)⁺.

Compound 6 was treated with six equivalents of sodium azide (NaN₃) in dimethylformamide (DMF) at 100° C. for 1 h. The product was extracted with ethyl acetate (EtOAc) and washed with H₂O. The organic layer was concentrated under reduced pressure to give azide 7 as a white solid in 91% yield. When the reaction was performed on a scale greater than 200 mg the reaction time was increased to 1.5 h to ensure full consumption of starting material. The structure was confirmed using ¹H and ¹³C NMR spectroscopy. A noticeable downfield shift was observed in the ¹³C NMR spectrum for the CH₂N₃ (50.5 ppm) when compared to the CH₂Br (29.7 ppm) present in compound 6. HRMS provided further evidence of the product with a m/z of 440.1272 (M+Na)⁺ detected; calculated for C₁₆H₂₃N₃O₁₀ (M+Na)⁺.

Compound 7 was treated with five equivalents of sodium methoxide (NaOMe) in methanol (CH₃OH) at ambient temperature for 24 h. The reaction mixture was quenched by addition of Amberlite® (IR120 H⁺) cation exchange resin and filtered. Concentration of the filtrate under reduced pressure gave the product, 2-azidoethyl-α-D-mannose (8) as a white solid in 98% yield. Successful deacetylation was confirmed by the disappearance of acetate resonances in both the ¹H and ¹³C NMR spectra. HRMS confirmed the product by detection of m/z of 272.0852 (M+Na)⁺, calculated for C₈H₁₅N₃O₆ (M+Na)⁺.

Following a similar synthetic pathway, β-D-maltose octaacetate (X1) was glycosylated with 2-bromoethanol in the presence of BF₃.Et₂O at 0° C. The reaction was warmed to ambient temperature and stirring was maintained for 24 h. The product was purified using silica gel column chromatography using 30% EtOAc in hexane. Bromide (X2) was isolated as a white solid in 63% yield. Successful substitution of the C1-acetyl group was confirmed by NMR spectroscopy. An upfield shift of the H1 doublet from 5.66 to 4.59 ppm as well as the presence of a doublet of doublet of doublet at 4.12 ppm (OCH₂) and a multiplet at 3.43-3.46 ppm (CH₂Br) in the ¹H NMR spectrum confirmed successful glycosylation with 2-bromoethanol at the C1 position. HRMS further confirmed the product by detection of m/z of 765.1208 (M+Na)⁺, calculated for C₂₈H₃₉⁷⁹BrO₁₈ (M+Na)+.

Compound X2 was treated with six equivalents of NaN₃ in DMF at 100° C. for 1.5 h. The product was extracted with EtOAc and washed with H₂O. The organic layer was dried under reduced pressure to give azide X3 in 71% yield. Evidence of a bromide to azido substitution was observed with a downfield shift in the ¹³C spectrum from CH₂Br at 30.0 ppm to CH₂N₃ at 50.6 ppm. HRMS confirmed the product by detection of m/z of 728.2107 (M+Na)⁺, calculated for C₂₈H₃₉N₃O₁₈ (M+Na)⁺.

The deacetylation of X3 was carried out using six equivalents of NaOMe in CH₃OH at ambient temperature for 24 h. The reaction mixture was quenched by addition of Amberlite® (IR120 H⁺) cation exchange resin and filtered. Concentration of the filtrate under reduced pressure gave the product as a white solid in 98% yield. Successful deacetylation was confirmed by the disappearance of ¹H and ¹³C NMR resonances associated with the acetate groups present in the starting material. HRMS confirmed the product by detection of m/z of 434.1380 (M+Na)⁺, calculated for C₁₄H₂₅N₃O₁₁ (M+Na)⁺.

Following a similar synthetic pathway, β-D-glucose pentaacetate (XX1) was glycosylated with 2-bromoethanol in the presence of BF₃.Et₂O and a catalytic amount of zinc chloride (ZnCl₂) at 0° C. The reaction was warmed to ambient temperature and stirring was maintained for 24 h. The reaction was quenched with solid potassium carbonate (K₂CO₃), extracted with CH₂Cl₂ and washed with H₂O. The organic phase was concentrated to give a viscous syrup which was purified by crystallisation from 50% EtOAc in hexane to give bromide XX2 in a 25% yield. Successful substitution of the C1 acetyl group was confirmed by ¹H and ¹³C NMR spectroscopy. An upfield shift of the H1 doublet resonance from 5.71 to 4.57 ppm was observed in the ¹H NMR spectrum. Appearance of two methylene group resonances, the diastereotopic proton resonances of OCH₂ as multiplets at the ranges of 3.79-3.84 and 4.15-4.18 ppm, and that of CH₂Br as a multiplet at 3.46-3.75 ppm confirmed successful glycosylation of 2-bromoethanol. HRMS confirmed formation of the product with a detected m/z at 477.0366 (M+Na)⁺, calculated for C₁₆H₂₃⁷⁹BrO₁₀ (M+Na)⁺.

Compound XX2 was treated with three equivalents of NaN₃ in DMF at 100° C. for 1 h. The product was extracted with EtOAc and washed with H₂O. The organic layer was concentrated under reduced pressure to give compound XX3 as a white solid in 71% yield. Bromide to azido substitution was confirmed using ¹³C NMR spectroscopy by a downfield shift of the CH₂N₃ resonance (50.6 ppm) when compared to the CH₂Br (30.0 ppm) resonance present in starting material XX2. HRMS confirmed the successful formation of compound XX3 with a detected m/z at 440.1281 (M+Na)⁺, calculated for C₁₆H₂₃N₃O₁₀ (M+Na)⁺.

Deacetylation of compound XX3 was achieved using five equivalents of NaOMe in CH₃OH at ambient temperature for 24 h. The reaction mixture was quenched by addition of Amberlite® (IR120 H⁺) cation exchange resin and filtered. Concentration of the filtrate under reduced pressure gave compound XX4 as a white solid in 99% yield. Successful deacetylation was confirmed using ¹H and ¹³C NMR spectroscopy by the disappearance of resonances attributed to the acetate groups present in the starting material. HRMS detected a molecular ion at m/z=272.0852 (M+Na)⁺, calculated for C₁₆H₂₃N₃O₁₀ (M+Na)⁺.

Following a similar synthetic pathway, β-D-galactose pentaacetate (XXX1) was alkylated with 2-bromoethanol in the presence of BF₃.Et₂O and a catalytic amount of ZnCl₂ at 0° C. The reaction was warmed to ambient temperature and stirring was maintained for 24 h. The reaction was quenched by addition of solid K₂CO₃, then extracted with CH₂Cl₂ and rinsed with H₂O. The product was purified by silica gel column chromatography in 30% EtOAc in hexane to give compound XXX2 as white solid in 49% yield. Successful substitution of the C1 acetyl group was confirmed by ¹H and ¹³C NMR spectroscopy. An upfield shift of the H1 doublet resonance from a 5.34 to 4.46 ppm accompanied by corresponding loss of one COCH₃ resonance was observed in the ¹H NMR spectrum. Appearance of two methylene group resonances provided further evidence of the correct structure; diastereotopic proton resonances of OCH₂ were observed as multiplets at 3.70-3.75 and 4.00-4.09 ppm, and the CH₂Br protons were observed as a multiplet at 3.36-3.39 ppm. HRMS confirmed formation of the product with a detected m/z at 477.0364 (M+Na)⁺, calculated for C₁₆H₂₃⁷⁹BrO₁₀ (M+Na)⁺.

Compound XXX2 was treated with three equivalents of NaN₃ in DMF at 100° C. for 1 h. The product was extracted with EtOAc and washed with H₂O. The organic layer was concentrated under reduced pressure to give compound XX3 as a white solid in 75% yield. Evidence of successful substitution was obtained using 13C NMR spectroscopy; a downfield shift of the CH₂N₃ group (50.5 ppm) when compared to the CH₂Br (29.9 ppm) was observed. HRMS confirmed the successful formation of compound XXX3 with a detected m/z at 440.1294 (M+Na)⁺, calculated for C₁₆H₂₃N₃O₁₀ (M+Na)⁺.

Deacetylation of compound XXX3 was performed using five equivalents of NaOMe in CH₃OH at ambient temperature for 24 h. The reaction mixture was quenched by addition of Amberlite® (IR120 H⁺) cation exchange resin and filtered. Concentration of the filtrate under reduced pressure gave compound XXX4 as a white solid in 77% yield. Successful deacetylation was confirmed using ¹H and ¹³C NMR spectroscopy by the disappearance of resonances attributed to the acetate groups present in the starting material. HRMS detected a molecular ion at m/z=272.0854 (M+Na)⁺, calculated for C₁₆H₂₃N₃O₁₀ (M+Na)⁺.

Coordination of an azide functionalised mannose, maltose, glucose or galactose residue to a tricarbonyl phenanthroline (4-ethynylphenyltetrazolato) Rhenium(I) framework, was achieved using the reaction detailed in Scheme 2 as shown in FIG. 5.

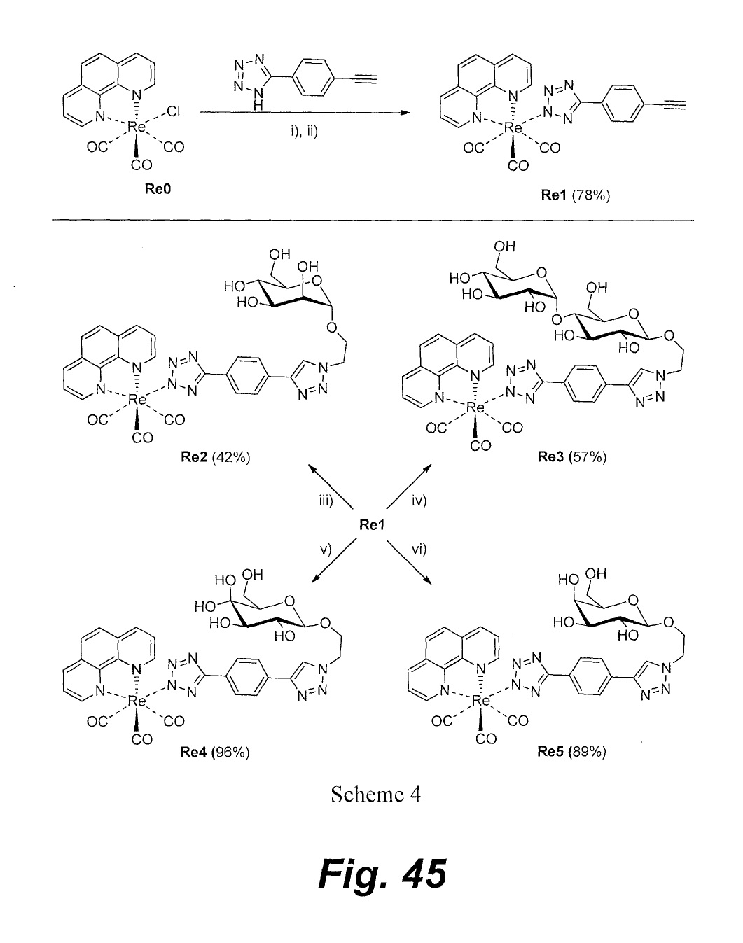

Reagents and conditions for Scheme 4 (FIG. 45): i) AgBF₄/CH₃CN/reflux/dark/4 h; ii) 5-(4-ethynylphenyl)-1H tetrazole/Et₃N/CH₃CN/reflux/24 h; iii) CuSO₄.5H₂O/NaAsc/DMF/H₂O/24 h; iv) CuSO₄.5H₂O/NaAsc/DMF/H₂O/24 h; v) CuSO₄.5H₂O/NaAsc/DMF/H₂O/72 h; vi) CuSO₄.5H₂O/NaAsc/DMF/H₂O/72 h.

The acetonitrile ligand (NCCH₃) was added to the parent complex Re0 (fac-[Re(phen)CO₃(Cl)]) using silver tetrafluoroborate (AgBF₄) in dry acetonitrile. The resulting complex (fac-[Re(phen)CO₃(NCCH₃)]) was then promptly exposed to the 5-(4-ethynylphenyl)-1H-tetrazole ligand and triethylamine, which promoted the exchange of NCCH₃ with the tetrazole ligand. By utilising column chromatography on neutral alumina, the starting materials were eluted with an ethyl acetate solvent system. The product (Re1) was isolated as a bright yellow solid in 78% yield.

Copper (Cu) catalysed alkyne azide cycloaddition was used to join together compound 8, to the alkyne functionalised Re(I) complex (Re1) by formation of a 1,4-triazole. The active Cu(I) catalyst was prepared by reduction of copper(II) sulphate pentahydrate with sodium ascorbate (NaAsc) under inert conditions. The successful reduction of Cu(II) to Cu(I) is essential to avoid the formation of the unwanted 1,5-regioisomer. Reaction was conducted in DMF/H₂O (5:1) stirred at ambient temperature, shielded from light, under inert conditions for 24 h. The reaction mixture was concentrated under reduced pressure and suspended in saturated NaHCO₃. The mixture was centrifuged and the crude product was isolated as a green waxy precipitate; this step was essential to remove residual copper in the supernatant. Excess 2-azidoethoxy-α-D-mannose (8) and residual NaHCO₃ was removed by suspending the precipitate in H₂O, centrifugation and discarding the supernatant. The material was then suspended in CH₂Cl₂ and centrifuged, the supernatant was decanted and discarded removing any remaining Re1. The precipitate was dried in vacuo to give Re2 (42%) as a bright yellow solid. Analysis by ¹H NMR spectroscopy revealed the appearance of one proton resonance as a singlet at 8.24 ppm, representing a single 1,4-triazole product. ESI-MS confirmed successful formation of the product with the detection of a molecular ion at m/z 873 (M+H)⁺.

The synthetic method for Re2 was then applied to attach the azido maltose residue X4 to the Re1 complex. An identical purification procedure was employed to remove any excess starting materials, isolating Re3 (Rhenium maltose complex) as a bright yellow solid in 57% yield. Analysis by ¹H NMR spectroscopy confirmed the formation of the desired 1,4-triazole product by presence of a single triazole proton resonance at 8.41 ppm, confirming isomeric purity. ESI-MS confirmed successful formation of the product with the detection of a molecular ion at m/z 1035 (M+H)⁺.

Re4 (Rhenium Glucose) and Re5 (Rhenium Galactose) were prepared by a similar method to Re2 and Re3, with the exception that a greater excess of azido sugar starting material was employed to ensure consumption of Re1. Consequently, the CH₂Cl₂ extraction was omitted from the work up procedure and higher yields were achieved. Re4 was obtained as a yellow solid in 96% yield and was confirmed to be isomerically pure by ¹H NMR spectroscopy; a single triazole proton resonance at 8.44 ppm was observed. ESI-MS provided further confirmation by the detection of a molecular ion at m/z=873 (M+H)⁺. Re5 was obtained as a yellow solid in 89% yield and was confirmed to be isomerically pure by ¹H NMR revealing a single triazole proton resonance at 8.45 ppm. ESI-MS gave further confirmation by the detection of a molecular ion at m/z=873 (M+H)⁺.

Each Rhenium complex; Re2 (Rhenium mannose), Re3 (Rhenium maltose), Re4 (Rhenium glucose), Re5 (Rhenium galactose) was then subjected to photophysical examination in aqueous solution in preparation for further investigation in live cells.

### Example 9—Photophysical Properties of Re4 and Re5

Photophysical properties of Re4 and Re5 in 50:50 MeOH/H₂O

Solutions of Re4 and Re5 in 1:1 MeOH/H₂O (5×10⁻⁶ M) with constant ionic strength (I=1×10⁻³ M using NaCl) were each titrated against NaOH. UV-visible absorption spectra were recorded over the range of 250-450 nm. Fluorescent measurements were recorded over the range of 510-740 nm at an excitation wavelength of 405 nm. Excitation was performed at 405 nm to ensure results were analogous to the 810 nm multiphoton laser excitation used for cellular investigations.

The photophysical properties of Re4 and Re5 in 50:50 MeOH/H₂O were similar to Re2 and Re3: the UV-visible spectra exhibited a prominent higher energy band at 275 nm resulting from the spin-allowed, ligand centred π-π* transition localised on the phenyltetrazolato-saccharide ligand. A lower energy charge transfer band in the 360-380 nm region was ascribed to a metal-ligand-to-ligand charge transfer (MLLCT). The molar absorptivity of Re4 and Re5 only slightly increased with decreasing pH, over the pH range 4-8.

To further probe the photophysical properties of Re4 and Re5, two excitation wavelengths were examined; 275 and 405 nm. Excitation at 275 nm was used to activate the π-π* transition of the triazole moiety. Fluorescent emission from the singlet state of the triazole was observed at 345 nm. Upon decreasing the pH, this emission band underwent a hypsochromic shift to 317 nm for Re4 and 322 nm for Re5 accompanied by a drop in emission intensity. The pH responsive shifts in triazole emission at 350 nm was seemingly reversible for Re4 and Re5 (as evidenced by forward and back pH titration data. These results are consistent with the observed triazole emission of Re2 and Re3. Furthermore, there is evidence of a pKa at approximately pH=4.2 for both Re4 and Re5. Phosphorescent emission from the triplet ³MLCT state was observed at 560 nm, and remained stable within the pH range of 4-8.

An excitation wavelength of 405 nm was employed to emulate the 810 nm two-photon excitation used in cellular imaging experiments. The emission spectra of Re4 and Re5 were characteristic of phosphorescence from the triplet ³MLCT state at 555 nm. Emission at 555 nm remained stable within the pH range of 4-8. This property was consistent across all four compounds (Re2, Re3, Re4 and Re5).

Cellular imaging: Re4 has been applied to PNT1a and 22RV1 prostate cells, H9c2 cells and CHO cells and showed intracellular staining consistent with perinuclear staining. Re5 has been applied to H9c2 cells and also showed intracellular staining consistent with perinuclear staining. Re4 and Re5 are less soluble than Re2 and Re3 and hence, although further work is required for full cellular characterisation[SP1].

### Example 10—Kits for Cell Imaging

An example of a kit using complexes comprising a transition metal carbonyl complex, a conjugated bidentate ligand and a tetrazolato compound comprising a saccharide group for imaging of cells is described.

Kit Components

(i) A complex of a transition metal carbonyl compound, a conjugated bidentate ligand and a tetrazolato compound comprising a saccharide group, provided in solid form or dissolved in DMSO (eg 10 mM).

(ii) Dilution medium for addition of imaging agents/probes to cells—typically sterile PBS, sterile water or sterile cell culture medium without serum.

(iii) One or more other components, including positive and negative controls, such as positive and negative controls for a specific intracellular component, such as lysosomes.

(iv) Instructions for preparing imaging agents/probes, using the probes for intracellular imaging and/or detection of specific structures in cells.

Kits are to be stored at room temperature in a dark place. Working solutions of complexes are to be prepared only at the time of use. If the agents are provided in solid form, a stock solution is required. To prepare a 10 mM stock solution in DMSO: dissolve 1 mg of agent in 160 μl of DMSO. The 10 mM stock solution in DMSO (supplied or prepared by user) must then be diluted to a working solution concentration. Preparation of a typical working solution would require a 1/1000 or 1/500 dilution of the 10 mM stock solution in DMSO into PBS solution.

For tissues, the tissues are isolated in PBS (or other physiological media) and mounted on a coverslip. For cells, these are grown as per normal practice on coverslips.

The media is to be removed and replaced with 10-20 μM solution of probes in PBS (or appropriate physiological media/cell media) at a dilution of stock solution from 1/1000-1/500, for 15-30 minutes at physiologically appropriate temperature (37° C. for cell culture or 25° C. for insect larvae).

Samples are washed for one minute in PBS. If co-staining is performed, the samples are washed in PBS for 30 seconds, before incubating with counterstain.

Tissues are mounted in optical coupling gel, to prevent dehydration prior to imaging and to maintain tissue integrity. For cells, the coverslip is mounted in PBS for immediate imaging.

Samples are to be imaged immediately following staining.

Following fixation (paraffin embedding or alcohol based fixation) samples are washed three times for five minutes in PBS at room temperature.

Samples are incubated with 10-20 μM solution of the probe in PBS (1/1000-1/500 dilution of stock) for 20-30 minutes for alcohol or paraformaldehyde fixed samples, or 40 minutes to one hour for paraffin embedded tissue sections at room temperature, with agitation.

Samples are washed three times for five minutes in PBS at room temperature, with agitation and mounted in 80% glycerol for imaging. Samples can be stored overnight at room temperature in a dark cupboard.

Rhenium probes may be excited by a UV or blue light sources (eg 405 nm). Image collection is performed with a wideband pass filter within the range of 500-650 nm, or narrowband pass filter within this emission range. Photobleaching may occur with mercury light sources if multiple colour imaging is being performed, i.e. if the sample is excited at multiple wavelengths at once.

For two-photon and confocal microscopy, rhenium probes are excited at 800-830 nm using a two-photon pulse laser or 405 steady state laser and detection in the range of 490-670 nm, with an emission maxima at around 570.

Although the present disclosure has been described with reference to particular embodiments, it will be appreciated that the disclosure may be embodied in many other forms. It will also be appreciated that the disclosure described herein is susceptible to variations and modifications other than those specifically described. It is to be understood that the disclosure includes all such variations and modifications. The disclosure also includes all of the steps, features, compositions and compounds referred to, or indicated in this specification, individually or collectively, and any and all combinations of any two or more of the steps or features.

Also, it is to be noted that, as used herein, the singular forms “a”, “an” and “the” include plural aspects unless the context already dictates otherwise.

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

Reference to any prior art in this specification is not, and should not be taken as, an acknowledgment or any form of suggestion that this prior art forms part of the common general knowledge in any country.

The subject headings used herein are included only for the ease of reference of the reader and should not be used to limit the subject matter found throughout the disclosure or the claims. The subject headings should not be used in construing the scope of the claims or the claim limitations.

The description provided herein is in relation to several embodiments which may share common characteristics and features. It is to be understood that one or more features of one embodiment may be combinable with one or more features of the other embodiments. In addition, a single feature or combination of features of the embodiments may constitute additional embodiments.

All methods described herein can be performed in any suitable order unless indicated otherwise herein or clearly contradicted by context. The use of any and all examples, or exemplary language (e.g., “such as”) provided herein, is intended merely to better illuminate the example embodiments and does not pose a limitation on the scope of the claimed invention unless otherwise claimed. No language in the specification should be construed as indicating any non-claimed element as essential.

Future patent applications may be filed on the basis of the present application, for example by claiming priority from the present application, by claiming a divisional status and/or by claiming a continuation status. It is to be understood that the following claims are provided by way of example only, and are not intended to limit the scope of what may be claimed in any such future application. Nor should the claims be considered to limit the understanding of (or exclude other understandings of) the present disclosure. Features may be added to or omitted from the example claims at a later date.

Although the present disclosure has been described with reference to particular examples, it will be appreciated by those skilled in the art that the disclosure may be embodied in many other forms.

1.-49. (canceled)

50. A complex comprising a transition metal carbonyl compound, a conjugated bidentate ligand and a tetrazolato compound comprising a saccharide group.

51. The complex according to claim 50, wherein the transition metal carbonyl compound is a Re(I) tricarbonyl compound.

52. The complex according to claim 50, wherein the conjugated bidentate ligand comprises a bidentate diimine ligand.

53. The complex according to claim 50, wherein the conjugated bidentate ligand comprises a phenanthroline or a bipyridine compound.

54. The complex according to claim 50, wherein the conjugated bidentate ligand comprises a 1, 10 phenanthroline compound and/or a substituted derivative thereof.

55. The complex according to 50, wherein the tetrazolato compound comprises a phenyltetrazolate and/or a substituted derivative thereof.

56. The complex according to claim 50, wherein the tetrazolato compound comprises a 1,2,3 triazole phenyltetrazolate and/or a substituted derivative thereof.

57. The complex according to claim 50, wherein the tetrazolato compound comprises a monosaccharide group and/or a disaccharide group.

58. The complex according to claim 50, wherein the saccharide group comprises a mannopyranose group, a glucose group or a galactose group.

59. The complex according to claim 50, wherein the saccharide group comprises a maltose group.

60. The complex according to claim 50, wherein the complex has the following chemical structure: 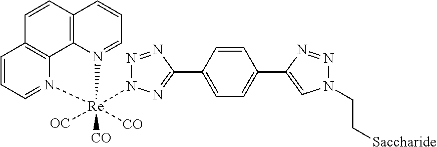
and/or a salt, a solvate, a tautomer, a stereoisomer, a substituted derivative, or an open chain form of the saccharide of the complex, wherein the saccharide group comprises one or more of a monosaccharide, a disaccharide, an oligosaccharide and a polysaccharide.

61. The complex according to claim 50, wherein the complex has one of the following chemical structures: 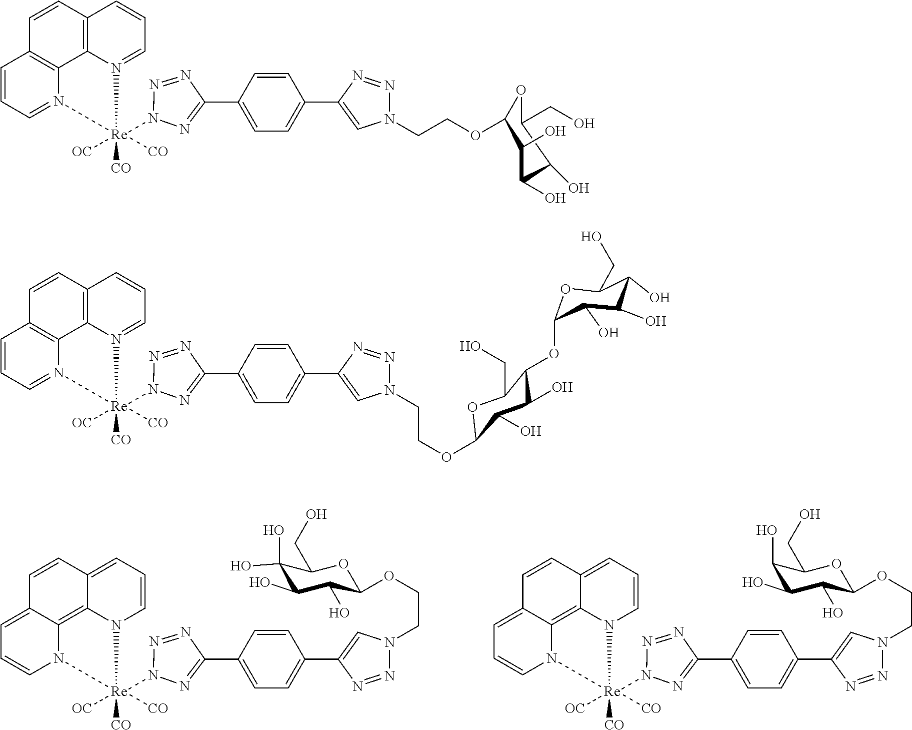
and/or a salt, a solvate, a tautomer, a stereoisomer, a substituted derivative thereof or an open chain form of the saccharide of any of the chemical structures.

62. An intracellular imaging agent, the agent comprising a complex according to claim 50.

63. A method of intracellular imaging of a cell, the method comprising exposing a cell to a complex according to claim 50 and imaging the complex in the cell.

64. The method according to claim 63, wherein the cell is a live cell.

65. The method according to claim 63, wherein the cell is present in vivo, in a cell sample, a sample of live cells, a cell extract, a biopsy, a bodily fluid sample, a blood sample, a urine sample, a saliva sample and/or an extract, component, derivative, processed form or purified form of any of the aforementioned.

66. The method according to claim 63, wherein the method is used for imaging of a live cell, in vivo imaging, to detect or label a cellular structure, to detect or label a vesicular compartment, to detect or label a lysosome, to detect or label a lysosome, to detect or label cytosol or a cytosolic structure in the cell, to detect or label endoplasmic reticulum, to detect or label a drosophila fat body cell, to detect or label a macrophage, to detect or label a myoblast, muscle cell or a myocardial cell, to detect or label a non-cancerous cell and/or a cancerous cell, to identify a non-cancerous cell or a cancerous cell, to screen for cancerous cells, and to distinguish a cancerous cell from a non-cancerous cell.

67. A method of identifying a cancerous prostate cell, the method comprising exposing the cell to a complex with the following chemical structure: 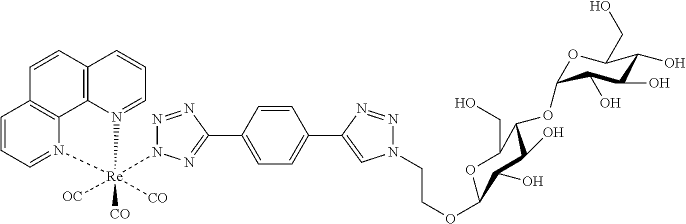
and/or a salt, a solvate, a tautomer, a stereoisomer, a substituted derivative, or an open chain form of the saccharide thereof, and identifying the cell as a cancerous prostate cell on the basis of altered labelling of the cell with the complex and/or altered localisation of the complex in the cell.

68. The method according to claim 67, wherein the method comprises identifying a cancerous prostate cell on the basis of increased labelling of the cell with the complex and/or altered localisation or distribution of the complex in the cell.

69. A kit for intracellular imaging of cells, the kit comprising a complex comprising a transition metal carbonyl compound, a conjugated bidentate ligand and a tetrazolato compound comprising a saccharide group.
